(19) United States
(12) Patent Application Publication
(10) Pub. No.: US 2005/0288299 A1
(43) Pub. Date: Dec. 29, 2005

(54) Azaindole derivatives as inhibitors of p38 kinase

(76) Inventors: Babu J. Mavunkel, Sunnyvale, CA (US); John J. Perumattam, Los Altos, CA (US); Qing Lu, Foster City, CA (US); Sundeep Dugar, San Jose, CA (US); Bindu Goyal, Fremont, CA (US); Dan X. Wang, Fremont, CA (US); Sarvajit Chakravarty, Mountain View, CA (US); Gregory R. Luedtke, Sunnyvale, CA (US); Imad Nashashibi, Mountain View, CA (US); Richland Tester, Alameda, CA (US); Xuefei Tan, Union City, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/107,027

(22) Filed: Apr. 15, 2005

Related U.S. Application Data
(63) Continuation-in-part of application No. 10/683,656, filed on Oct. 9, 2003.
(60) Provisional application No. 60/417,599, filed on Oct. 9, 2002.

(51) Int. Cl. A61K 31/496; A61K 31/454; C07D 487/02; C07D 43/02

(52) U.S. Cl. 514/253.04; 514/254.06; 514/322; 544/362; 544/366; 546/199

(57) ABSTRACT

The invention is directed to methods to inhibit p38 kinase, preferably p38-α using compounds which are azaindoles wherein the azaindoles are coupled through a piperidine or piperazine type linker to another cyclic moiety.

### RELATED APPLICATIONS

This application is a continuation in part of application Ser. No. 10/683,656, which was filed Oct. 9, 2003 and claimed priority to U.S. Provisional Patent Application No. 60/417,599, filed Oct. 9, 2002, each of which is hereby incorporated by reference in its entirety.

### FIELD OF THE INVENTION

The invention relates to compounds, compositions, and methods for treating various disorders associated with undesirably high activity of p38 kinase. More specifically, it concerns compounds that are related to azaindole coupled through an azacyclic moiety to an aryl group, which are useful in these methods.

### BACKGROUND ART

A large number of chronic and acute conditions have been recognized to be associated with perturbations of the inflammatory response. Many different cytokines are known to participate in this response, including IL-1, IL-6, IL-8 and TNF. It appears that the activity of these cytokines in the regulation of inflammation rely at least in part on the activation of an enzyme in the cell signaling pathway, a member of the MAP kinase family generally known as p38, and alternatively known as CSBP and RK. This kinase is activated by dual phosphorylation after stimulation by physiochemical stress, treatment with lipopolysaccharides or with proinflammatory cytokines such as IL-1 and TNF. Therefore, inhibitors of the kinase activity of p38 are useful anti-inflammatory agents.

Within the last several years, p38 has been shown to comprise a group of MAP kinases designated p38-α, p38-β, p38-γ and p38-δ. Jiang, Y., et al., J. Biol. Chem. (1996) 271:17920-17926 reported characterization of p38-β as a 372-amino acid protein closely related to p38-α. In comparing the activity of p38-α with that of p38-β, the authors state that while both are activated by proinflammatory cytokines and environmental stress, p38-β was preferentially activated by MAP kinase kinase-6 (MKK6) and preferentially activated transcription factor 2, thus suggesting that separate mechanisms for action may be associated with these forms.

Kumar, S., et al., Biochem. Biophys. Res. Comm. (1997) 235:533-538 and Stein, B., et al., J. Biol. Chem. (1997) 272:19509-19517 reported a second isoform of p38-β, p38-β2, containing 364 amino acids with 73% identity to p38-α. All of these reports show evidence that p38-β is activated by proinflammatory cytokines and environmental stress, although the second reported p38-β isoform, p38-β2, appears to be preferentially expressed in the CNS, heart and skeletal muscle compared to the more ubiquitous tissue expression of p38-α. Furthermore, activated transcription factor-2 (ATF-2) was observed to be a better substrate for p38-β2 than for p38-α, thus suggesting that separate mechanisms of action may be associated with these forms. The physiological role of p38-β1 has been called into question by the latter two reports since it cannot be found in human tissue and does not exhibit appreciable kinase activity with the substrates of p38-α.

The identification of p38-γ was reported by Li, Z., et al., Biochem. Biophys. Res. Comm. (1996) 228:334-340 and of p38-δ by Wang, X., et al., J. Biol. Chem. (1997) 272:23668-23674 and by Kumar, S., et al., Biochem. Biophys. Res. Comm. (1997) 235:533-538. The data suggest that these two p38 isoforms (γ and δ) represent a unique subset of the MAPK family based on their tissue expression patterns, substrate utilization, response to direct and indirect stimuli, and susceptibility to kinase inhibitors.

PCT applications WO98/06715, WO98/07425, and WO 96/40143, all of which are incorporated herein by reference, describe the relationship of p38 kinase inhibitors with various disease states. As mentioned in these applications, inhibitors of p38 kinase are useful in treating a variety of diseases associated with chronic inflammation. These applications list rheumatoid arthritis, rheumatoid spondylitis, osteoarthritis, gouty arthritis and other arthritic conditions, sepsis, septic shock, endotoxic shock, Gram-negative sepsis, toxic shock syndrome, asthma, adult respiratory distress syndrome, stroke, reperfusion injury, CNS injuries such as neural trauma and ischemia, psoriasis, restenosis, cerebral malaria, chronic pulmonary inflammatory disease, chronic obstructive pulmonary disease, cystic fibrosis, silicosis, pulmonary sarcosis, bone fracture healing, bone resorption diseases such as osteoporosis, soft tissue damage, graft-versus-host reaction, Crohn's Disease, ulcerative colitis including inflammatory bowel disease (IBD) and pyresis.

The above-referenced PCT applications disclose compounds which are p38 kinase inhibitors said to be useful in treating these disease states. These compounds are either imidazoles or are indoles substituted at the 3- or 4-position with a piperazine ring linked through a carboxamide linkage. Additional compounds which are conjugates of piperazines with indoles are described as insecticides in WO97/26252, also incorporated herein by reference.

Certain aroyl/phenyl-substituted piperazines and piperidines which inhibit p38-α kinase are described in PCT publication WO00/12074 published 9 Mar. 2000. In addition, indolyl substituted piperidines and piperazines which inhibit this enzyme are described in PCT publication No. WO99/61426 published 2 Dec. 1999. Carbolene derivatives of piperidine and piperazine as p38-α inhibitors are described in PCT publication WO 00/59904 published 12 Oct. 2000. Additional substitutions on similar compounds are described in PCT publication WO 00/71535 published 30 Nov. 2000. The disclosure of these documents is incorporated herein by reference.

### DISCLOSURE OF THE INVENTION

The invention is directed to methods and compounds useful in treating conditions that are characterized by excessive, or undesirably high, p38 kinase activity. As used herein, p38 kinase, sometimes shortened to “p38”, refers to all of the isoforms having p38 kinase activity. The conditions characterized by undesirably high p38 kinase activity include those identified above, particularly inflammation related disorders such as arthritis. In addition, p38 activity has been associated with pain, cardiovascular diseases such as acute coronary syndrome, osteolytic lesions and other cancers, myelodysplasia and multiple myeloma. The compounds of the invention are useful in treating and alleviating these and other disorders as further described below.

Compounds of the invention have been found to inhibit the various forms of p38 kinase, particularly the α-isoform, and are thus useful in treating conditions mediated by these activities. The compounds of the invention are of the formula (1),

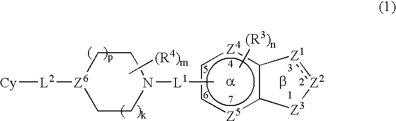

and the pharmaceutically acceptable salts thereof, wherein:

[symbol] represents a single or double bond;

one of Z¹ and Z² is CQ or CR¹Q and the other of Z¹ and Z² is CR¹ or C(R¹)₂;

Q can be any of the groups that R¹ can represent, or Q can be WᵢC(═O)XⱼY, wherein
each of W and X is independently an alkylene, alkenylene, alkynylene, or heteroalkylene linker up to four atoms in length, each of which is optionally substituted with one or more C1-C4 alkyl, C1-C4 heteroalkyl, halo, CN, COOR, ═O, ═NR, ═NOR, ═N—CN, OR, and NR₂, wherein each R is independently H, C1-C4 alkyl, or C1-C4 heteroalkyl, and wherein two R can optionally cyclize to form a 3-7 membered ring containing 0-2 heteroatoms selected from N, O and S;
each of i and j is independently 0 or 1; and
Y is COR² or an isostere thereof;

Z³ is NR⁷, O, or S;

Z⁴ and Z⁵ are independently N, CH or CR³, or one of Z⁴ and Z⁵ can be a carbon to which L¹ is linked,
provided that at least one of Z⁴ and Z⁵ is N;

Z⁶ is N or CR⁵;

each of L¹ and L² is an alkylene, alkenylene, alkynylene, or heteroalkylene linker up to four atoms in length, which is optionally substituted with one or more C1-C4, alkyl, C1-C4 heteroalkyl, halo, CN, COOR, ═O, ═NR, ═NOR, ═N—CN, OR, or NR₂, wherein each R is independently H, C1-C4 alkyl, or C1-C4 heteroalkyl, and wherein two R can optionally cyclize to form a 3-7 membered ring containing 0-2 heteroatoms selected from N, O and S;

Cy is a cyclic group having 3-7 ring members that is substituted with 0-5 substituents R⁶, wherein two R⁶ substituents can form a ring that is fused to Cy, or
Cy can represent two cyclic groups having 3-7 ring members each, where both cyclic groups are bonded to a single atom of L² and where each of the two cyclic groups is optionally substituted with 0-5 substituents R⁶;

each R³ and R⁶ independently represents an optionally substituted C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
   wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
   and wherein two R′ can be linked to form a 3-7 membered ring optionally containing up to three heteroatoms selected from N, O and S;

each R¹, R², R⁵, and R⁷ independently represents H or one of the groups set forth for R³;

each R⁴ represents one of the groups set forth for R³, or it can be ═CR′₂, ═O, ═N—CN, ═N—OR′, or ═NR′, and two R⁴ can be linked to form a fused ring, spiro-fused ring, or bridging ring having 3-7 members;
wherein each R′ is independently H, C1-C6 alkyl, C1-C6 heteroalkyl, C1-C6 acyl, C2-C6 heteroacyl, C6-C10 aryl or C5-C10 heteroaryl;

each of p and k is an integer from 0-2 wherein the sum of p and k is 0-3;

n is 0-2; and

m is 0-4.

### MODES OF CARRYING OUT THE INVENTION

The compounds of formula (1) are useful in treating conditions which are characterized by excessive, or undesirably high, activity of p38 kinase, in particular the α-isoform. Conditions “characterized by excessive p38 activity” include those where this enzyme is present in increased amount, or where the enzyme has been modified to increase its inherent activity, or both, as well as conditions where the enzyme activity or level is not abnormally high but a medical benefit can be provided to a subject by reducing the subject's p38 kinase activity. Thus, “excessive activity” refers to any condition wherein the effectiveness or activity of these proteins is undesirably high, regardless of the cause.

The compounds of the invention are useful in conditions where p38 kinase exhibits excessive activity. These conditions are those in which fibrosis and organ sclerosis are caused by, or accompanied by, inflammation, oxidation injury, hypoxia, altered temperature or extracellular osmolarity, conditions causing cellular stress, apoptosis or necrosis. These conditions include ischemia-reperfusion injury, congestive heart failure, progressive pulmonary and bronchial fibrosis, hepatitis, arthritis, inflammatory bowel disease, glomerular sclerosis, interstitial renal fibrosis, chronic scarring diseases of the eyes, bladder and reproductive tract, bone marrow dysplasia, chronic infectious or autoimmune states and traumatic or surgical wounds. These conditions, of course, would be benefited by compounds which inhibit p38. Methods of treatment with the compounds of the invention are further discussed below.

### THE INVENTION COMPOUNDS

As used herein, “hydrocarbyl residue” refers to a residue which contains only carbon and hydrogen. The residue may be aliphatic or aromatic, straight-chain, cyclic, branched, saturated or unsaturated, or any combination of these. The hydrocarbyl residue, when so stated however, may contain heteroatoms in addition to or instead of the carbon and hydrogen members of the hydrocarbyl group itself. Thus, when specifically noted as containing heteroatoms the hydrocarbyl group may contain heteroatoms within the “backbone” of the hydrocarbyl residue, and when optionally substituted, the hydrocarbyl residue may also have one or more carbonyl groups, amino groups, hydroxyl groups and the like in place of one or more hydrogens of the parent hydrocarbyl residue.

As used herein, “inorganic residue” refers to a residue that does not contain carbon. Examples include, but are not limited to, halo, hydroxy, NO₂ or NH₂.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C1-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C1-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C1-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” or “heteroatom-containing form” as used herein refers to a derivative of a group such as an alkyl, aryl, or acyl, wherein at least one carbon atom of the designated hydrocarbyl group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

The compounds of the invention are derivatives of azaindoles: they are analogs of indole having at least one extra ring nitrogen, which is in the 6-membered ring. Thus they include ring systems where the ring labeled α in formula (1) is a pyridine or pyrazine ring, which is fused to a pyrrole or a partially saturated pyrrole ring that is labeled β in formula (1). For present purposes, the positions of atoms in the α and β rings will be described using the numbering shown in formula (1).

With respect to the ring labeled α, it is a pyridine or pyrazine ring. Where it is a pyridine ring, either Z⁴ or Z⁵ is N, and the other one of Z⁴ and Z⁵ is either CH or CR³, or it is C to which L¹ is attached. In some preferred embodiments, the ring labeled α is a pyrazine, so both Z⁴ and Z⁵ are N. In other favored embodiments, Z⁴ is CH and Z⁵ is N; in still others, Z⁴ is N and Z⁵ is CH. In these embodiments, it is sometimes preferred that L¹ is CO and it is sometimes preferred that L² is CH₂. Some of these preferred embodiments further include having Z⁶=N or having Z⁶=CH. Some of these embodiments further include a substituent R³ at position 6 on the ring labeled α. Some of these preferred embodiments have Z¹=CQ and Z²=CH or CMe. In some of these, Z³ is NR⁷.

The α ring of the azaindole is necessarily substituted with L¹ at one of positions 4, 5, 6, and 7. In some of the preferred embodiments, L¹ is attached at position 5, and in others it is at position 6. Position 5 is more preferred as the attachment point for L¹. Where both Z⁴ and Z⁵ are N, L¹ must attach at position 5 or position 6; it is often preferably attached at position 5 in these embodiments.

It is preferred that the bond connecting Z¹ to Z² represents a double bond; in such embodiments, one of Z¹ and Z² is CQ and the other is CR¹. Typically, Z¹ is CQ and Z² is CR¹ in such embodiments. However, compounds which contain a partially saturated β ring where the bond connecting Z¹ to Z² is a single bond are also included within the scope of the invention, and can often be made from the compounds having a double bond by, for example, reduction to introduce two hydrogen atoms; reduction followed by alkylation to introduce one H and one other substituent such as alkyl or alkylthio; or oxidation to introduce a carbonyl at Z², optionally followed by further functionalization to introduce a new substituent at Z¹. In such compounds, one of Z¹ and Z² is CR¹Q, and the other is CR¹₂; frequently in such embodiments at least one R¹ is H or C1-C4 alkyl.

R¹ can be H or an optionally substituted C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl. Preferred embodiments of R¹ include hydrogen, alkyl, aryl, arylalkyl, acyl, aroyl, heteroaryl, heteroalkyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-COOR, alkyl-OOCR, SO₃R, CONR₂, SO₂NR₂, NRSO₂NR₂, CN, CF₃, and NO₂, wherein each R is independently H, C1-8 alkyl, C1-8 alkenyl, C1-C8 acyl or C5-12 aryl or C6-C12 arylalkyl or heteroforms of any of these, and two R can optionally be linked to form a 3-7 membered ring containing one or more heteroatoms selected from N, O and S and optionally substituted as described for R. Similarly, when more than one R¹ is present, two R¹ can be joined to form a fused, optionally substituted aromatic or nonaromatic, saturated or unsaturated ring which contains 3-8 members. H or methyl is often preferred for R¹.

Q can be the same groups R¹ can represent, but often Q is preferably a polar group. These polar embodiments of Q include forms of R¹ which contain multiple heteroatoms, such as those comprising an amide or ester or sulfonyl group, and those containing a basic amine group, especially when it is in combination with at least one other heteroatom. Furthermore, in some preferred embodiments, Q is —Wᵢ—COXⱼY wherein Y is COR² or an isostere thereof, and R² is as defined below; each of W and X is an alkylene, alkenylene, alkynylene, or heteroalkylene linker up to four atoms in length, and optionally substituted with the typical substituents appropriate for such groups; and each of i and j is independently 0 or 1. Typically, each of W and X is an alkylene, alkenylene, alkynylene, or heteroalkylene linker up to four atoms in length, which is optionally substituted with one or more C1-C4 alkyl, C1-C4 heteroalkyl, halo, CN, COOR, ═O, ═NR, ═NOR, ═N—CN, OR, or NR₂, wherein each R is independently H, C1-C4 alkyl, or C1-C4 heteroalkyl, and wherein two R can optionally cyclize to form a 3-7 membered ring containing 0-2 heteroatoms selected from N, O and S.

Typically, the Z¹-Z² bond is a double bond, and R¹ is H, or alkyl, such as methyl, and Q is a polar group. “Polar group” in this context refers to an optionally substituted alkyl, alkenyl, alkynyl, or acyl, group, or a heteroform of one of these, that contains two or more heteroatoms selected from N, O and S. Such polar groups include alkyl substituted with an amide or ester or carbamate or urea, for example, and groups containing an amine nitrogen and at least one other heteroatom, and those containing a sulfonyl or sulfoxide. In some preferred embodiments, Q is —Wᵢ—COXⱼY as defined herein. In such embodiments, it is sometimes preferred that at least one of i and j is zero, and in some preferred embodiments both i and j are zero, so Q represents —C(═O)Y or —C(═O)C(═O)R².

In some particularly preferred embodiments of the invention, Q represents —Wᵢ—COXⱼY wherein Y is COR² or an isostere thereof, and R² is hydrogen or a suitable substituent as described herein; each of W and X is an alkylene, alkenylene, alkynylene, or heteroalkylene linking group up to four atoms in length and is optionally substituted, and each of i and j is independently 0 or 1. Each of W and X may be, for example, optionally substituted alkylene or heteroalkylene. Preferably, W and X are unsubstituted alkylenes. Preferably, j is 0 so that the carbonyl group is adjacent to Y, which is a carbonyl or isostere thereof, and X is absent. Preferably, also, i is 0 so that W is absent, and the proximal CO of the group is adjacent to the ring. However, compounds wherein the proximal CO is spaced from the ring can readily be prepared by selective reduction of a glyoxal substituted β ring, which can be prepared as exemplified herein, and compounds having j other than 0 are readily prepared by the same acylation reaction used to introduce a glyoxal moiety.

In certain preferred embodiments of the invention, the dotted line bond between Z¹ and Z² often represents a double bond, and frequently Z¹ is CQ and Z² is CR¹. Typically in these embodiments, Q is a polar group, and preferably Q is WᵢCOXⱼY as defined above. More preferably, Q is COY or COCOR², where R² is as defined below. R¹ in these embodiments is often H or CH₃, with R¹═H being more preferred.

In some preferred embodiments, as already mentioned, Q is COCOR². The substituent represented by R² in these embodiments, when R² is other than H, may be a hydrocarbyl residue (1-20C) containing 0-5 heteroatoms selected from O, S and N or an inorganic residue. Thus R² can be an optionally substituted C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl.

Preferred are embodiments wherein R² is straight or branched chain alkyl, alkenyl, alkynyl, aryl, arylalkyl, heteroalkyl, heteroaryl, or heteroarylalkyl, each optionally substituted with halo, alkyl, heteroalkyl, SR, SO₂R, SO₂NR₂, OR, NR₂, OCOR, NRCOR, NRCONR₂, NRSO₂R, NRSO₂NR₂, OCONR₂, or CONR₂, wherein each R is independently H, or C1-C8 alkyl, C1-C8 alkenyl, C1-C8 acyl, or C5-C10 aryl or the heteroatom-containing forms thereof.

Other preferred embodiments include those wherein R² is OR, NR_{2,} SR, NRCONR₂, OCONR₂, or NRSO₂NR₂ wherein each R is independently H, C1-C8 alkyl, C1-C8 alkenyl, C1-C8 acyl, or C5-C10 aryl or the heteroatom-containing forms thereof, and wherein two R may be linked together to form a 3-8 member ring which may contain up to three heteroatoms selected from N, O and S, and wherein said ring may further be substituted by C1-6 alkyl, heteroalkyl, alkenyl, or alkynyl, or C6-12 aryl, arylalkyl, heteroaryl, or heteroarylalkyl, each of which is optionally substituted, or by halo, SR, SO₂R, SO₂NR₂, OR, NR₂, OCOR, NRCOR, NRCONR₂, NRSO₂R, NRSO₂NR₂, or OCONR₂, wherein each R is independently H, C1-C8 alkyl, C1-C8 alkenyl or C5-C12 aryl or arylalkyl, or the heteroatom-containing forms of one of these, and wherein two R may cyclize to form a 3-8 member ring, optionally substituted with the substituents described for R.

Especially preferred embodiments of R² are heteroarylalkyl, —NR₂, heteroaryl, —COOR, —NRNR₂, heteroaryl-COOR, heteroaryloxy, —OR, heteroaryl-NR₂, —NROR and C1-C8 alkyl or heteroalkyl. Most preferably R² is an NR₂ group that is selected from the following: isopropyl piperazine, methyl piperazine, methylamine, dimethylamine, ethylamine, N-methyl-N-methoxyamine, pyrrolidine, isopropylamine, methoxyamine, piperazine, isobutyl carboxylate, oxycarbonylethyl, benzimidazolyl, aminoethyldimethylamine, isobutyl carboxylate piperazine, oxypiperazine, ethylcarboxylate piperazine, methyl, amine, aminoethyl pyrrolidine, aminopropanediol, piperidine, pyrrolidinyl-piperidine, pyrrolidine, pyrrolidinone, pyrroline, or methyl piperidine, wherein each ring listed may optionally be substituted with the substituents described above for R groups comprising R². Methoxy, ethoxy, hydroxy are also sometimes preferred embodiments of R².

Y can also represent an isostere of COR². Isosteres of COR² as represented by Y have varying lipophilicity and may contribute to enhanced metabolic stability. Thus, Y, as shown, may be replaced by the isosteres in Table A.

TABLE A

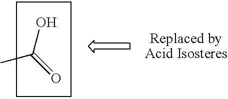

Acid Isosteres

| Names | | |
|---|---|---|
| of Groups | Chemical Structures | Substitution Groups (SG) |
| tetrazole | 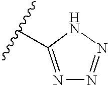 | n/a |
| 1,2,3-triazole | 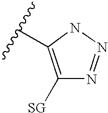 | H; SCH₃; COCH₃; Br; SOCH₃; SO₂CH₃; NO₂; CF₃; CN; COOMe |
| 1,2,4-triazole | 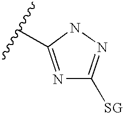 | H; SCH₃; COCH₃; Br; SOCH₃; SO₂CH₃; NO₂ |
| imidazole | 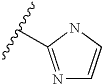 | H; SCH₃; COCH₃; Br; SOCH₃; SO₂CH₃; NO₂ |

Thus, isosteres include optionally substituted tetrazole, 1,2,3-triazole, 1,2,4-triazole and imidazole groups such as those shown in Table A, which may further be substituted on N by an optionally substituted alkyl or heteroalkyl group, preferably methyl or methoxymethyl.

As indicated by the foregoing descriptions, the bicyclic ‘azaindole’ portions of some of the preferred embodiments of the invention have one of the following formulas:

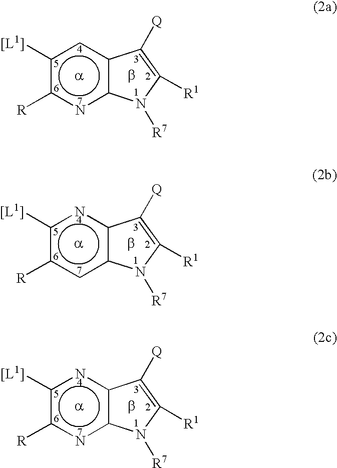

In the formulas (2a), (2b), and (2c), Q, R¹, and R⁷ have the meanings provided herein; R is either H or R³, where R³ is as described above; and [L¹] indicates a preferred attachment point for L¹ of formula (1). These formulas are presented to enhance clarity in describing certain preferred embodiments, and are not limitations on the scope of the invention.

L¹ links the azaindole ring system to another cyclic group, referred to herein as an azacyclic group, which contains N and Z⁶ and is further described below. The azacyclic group is then further linked by L², which attaches at Z⁶ in the azacyclic ring, to another cyclic group, Cy. Each of L¹ and L² is a linking group that comprises up to four serially connected atoms selected from C, N, O and S, and each is optionally substituted. Typically, L¹ and L² are C1-C4 alkylene, or heteroalkylene groups containing 1 or 2 heteroatoms in the chain, which are also optionally substituted. Suitable substituents are those set forth above as substituents for alkyl groups generally, and include but are not limited to, a moiety selected from the group consisting of alkyl, alkenyl, alkynyl, aryl, arylalkyl, acyl, aroyl, heteroaryl, heteroalkyl, heteroalkenyl, heteroalkynyl, heteroarylalkyl, NH-aroyl, arylacyl, heteroarylacyl, halo, ═O, ═NOR, OR, NR₂, SR, SOR, SO₂R, OCOR, NRCOR, NRCONR₂, NRCOOR, OCONR₂, RCO, COOR, alkyl-COOR, alkyl-OOCR, SO₃R, CONR₂, SO₂NR₂, NRSO₂NR₂, CN, CF₃, and NO₂, wherein each R is independently H, C1-6 alkyl, C2-6 alkenyl or C5-12 aryl or arylalkyl, or heteroforms of any of these, wherein two substituents on one atom can be joined to form a carbonyl moiety or an oxime, oximeether, oximeester or ketal of said carbonyl moiety.

In preferred embodiments, L¹ and L² are each an alkylene, alkenylene, alkynylene, or heteroalkylene linker up to four atoms in length, which is optionally substituted with one or more C1-C4 alkyl, C1-C4 heteroalkyl, halo, CN, COOR, ═O, ═NR, ═NOR, ═N—CN, OR, or NR₂, wherein each R is independently H, C1-C4 alkyl, or C1-C4 heteroalkyl, and wherein two R can optionally cyclize to form a 3-7 membered ring containing 0-2 heteroatoms selected from N, O and S. Preferred substituents for L¹ and L² include C1-C4 alkyl, especially methyl, and carbonyl (═O) and oxime (═NOR).

Typical, but nonlimiting, embodiments of L¹ and L² are (CH₂)₁₋₃(CO)₀₋₁ and CO(CH₂)₀₋₃, especially CH₂, CO, and isosteres of these, including forms where the carbonyl has been converted into an oxime, an oximeether, an oximeester, or a ketal, or optionally substituted isosteres, or longer chain forms. L2, in particular, may be alkylene, preferably 1-4C, or alkenylene, preferably 1-4C (a C1 alkenylene refers here to a C that is attached by a double bond to Z⁶, and thus requires Z⁶ to be C), and is optionally substituted with the substituents set forth above. Furthermore, L¹ or L² may be or may include a heteroatom such as N, S or O. In some preferred embodiments, L² is CH₂ and in others it is (CH₂)₂ or CH₂CO. In other preferred embodiments, L² is NR or S or it is CR₂, where each R is independently H, C1-C4 alkyl, or C1-C4 heteroalkyl, and where CR₂ can represent a 3-7 membered nonaromatic ring, optionally containing 1-2 heteroatoms selected from N, O and S.

L¹ can also be C1-C4 alkylene but is typically CO(CH₂)₀₋₃, which is optionally substituted. In many of the preferred embodiments of the invention, L¹ is CO, while in others it is COCH₂ or an isostere of CO. Isosteres of CO and CH₂, include S, SO, SO₂, CH—CN, C═NOR, and CHOH.

For L¹, CO is sometimes preferred and for L² methylene (CH₂) or substituted methylene, especially C1-C4 alkyl-substituted methylene such as CHMe or CMe₂, is sometimes preferred. In some preferred embodiments, L¹ is CO while L² is CH₂, and in such embodiments Z⁶ is sometimes preferably N, and in other such embodiments Z⁶ is preferably CH. Often in these embodiments, both k and p are 1; and Z¹ is CQ. In such embodiments, Z³ is typically NR⁷ and Cy is typically a mono-substituted or unsubstituted phenyl group. In such embodiments, Z² is often CH or CMe.

Between L¹ and L² is an azacyclic moiety of the following formula:

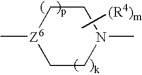

In this azacyclic group, Z⁶ is N or CR⁵ wherein R⁵ is H or a suitable substituent. Each of p and k is an integer from 0-2 wherein the sum of p and k is 0-3. The substituents R⁵ include, without limitation, halo, alkyl, alkoxy, aryl, arylalkyl, aryloxy, heteroaryl, acyl, carboxy, amino, mono- and di-alkylamino, and hydroxy. Preferably, R⁵ is one of the groups set forth above for R¹. Additionally, R⁵ can be joined with an R⁴ substituent or a substituent on L² to form an optionally substituted non-aromatic saturated or unsaturated hydrocarbyl ring which contains 3-8 members and 0-3 heteroatoms such as O, N and/or S. Preferred embodiments include compounds wherein Z⁶ is CH and those where it is N, and in such embodiments, both p and k are sometimes preferably 1, so the azacyclic ring is a piperidine or piperazine ring. However, rings of other sizes as well as bridged rings and bicyclic systems are included within the scope of the invention.

R⁴ may occur ‘m’ times on the azacyclic ring, where m is an integer of 0-4. In some preferred embodiments, m is 0. In others, m is 1, and R⁴ is frequently C1-C8 alkyl or C1-8 heteroalkyl, especially methyl, or it is ═O or COOR, where R is H or C1-C8 alkyl. In other preferred embodiments, m is 2, and the groups R⁴, which may be the same or different, are often both C1-8 alkyl groups. In some preferred embodiments, m is 2 and each R⁴ is a methyl group. In such embodiments, the two R⁴ groups are preferably in a trans orientation relative to each other, and they are typically positioned at least two atoms apart on the azacyclic ring. Thus some preferred embodiments of the azacyclic ring, when named assuming that L¹ is attached at position 1 of the azacyclic ring, include 2-methyl piperazine, 2R-methylpiperazine, 3-methyl piperazine, 3S-methylpiperazine, 2R,5S-dimethylpiperazine, 2-piperazinone, 3-piperazinone, and the piperidines that correspond to each of these named piperazines, having Z⁶=CH.

R⁴ represents a suitable substituent for an alkyl group, such as a hydrocarbyl residue (1-20C) containing 0-5 heteroatoms selected from O, S and N. Preferably R⁴ is alkyl, alkoxy, aryl, arylalkyl, aryloxy, heteroalkyl, heteroaryl, heteroarylalkyl, RCO, acyl, halo, CN, ═O, ═NOR, OR, NRCOR, NR₂, wherein R is H, alkyl (preferably 1-4C), aryl, or hetero forms thereof. Each appropriate substituent is itself unsubstituted or substituted with 1-3 substituents. The substituents are preferably independently selected from a group that includes alkyl, alkenyl, alkynyl, aryl, arylalkyl, acyl, aroyl, heteroaryl, heteroalkyl, heteroalkenyl, heteroalkynyl, heteroalkylaryl, NH-aroyl, halo, OR, NR₂, SR, SOR, SO₂R, OCOR, NRCOR, NRCONR₂, NRCOOR, OCONR₂, RCO, COOR, alkyl-COOR, alkyl-OOCR, SO₃R, CONR₂, SO₂NR₂, NRSO₂NR₂, CN, CF₃, R₃Si, and NO₂, wherein each R is independently H, C1-6 alkyl, C2-6 alkenyl or C5-12 aryl or arylalkyl, or heteroforms of any of these, and wherein two of R⁴ on the same or adjacent positions can be joined to form a fused or spirofused, optionally substituted aromatic or nonaromatic, saturated or unsaturated ring which contains 3-8 members. Alternatively, R⁴ can be ═O or an oxime, oximeether, oximeester or ketal thereof.

Preferred embodiments of R⁴ comprise alkyl (1-4C), straight chain or branched, and where m is 2, R⁴ is often two alkyl substituents which may be further substituted. Most preferably m is 2, and the two R⁴ groups comprise two methyl groups at positions 2 and 5 or positions 3 and 6 of a piperidine or piperazine ring. These substituted forms of the azacyclic group may be chiral and an isolated enantiomer may be preferred.

Certain preferred embodiments of this azacyclic portion include a piperazine or piperidine having a 2,5-disubstitution pattern when the N to which L¹ is attached is defined as position 1, and the substituents are preferably in a trans orientation relative to each other. For example, 2R,5S-dimethylpiperazine, where Z⁶ is defined as position 4 for the purpose of numbering the ring atoms, and the 2,5-dimethylpiperidine having the same absolute and relative stereochemistry as 2R,5S-dimethyl piperazine are preferred forms of this moiety, as are the corresponding racemic versions of these groups. (The absolute stereochemistry defined in the corresponding piperidine may change depending on what L² is; so its stereochemistry is best defined by reference to the piperazine stereochemistry.)

Based on the foregoing description, in some of the preferred embodiments of the compounds of the invention, the azacyclic ring containing Z⁶ is often one of the following:

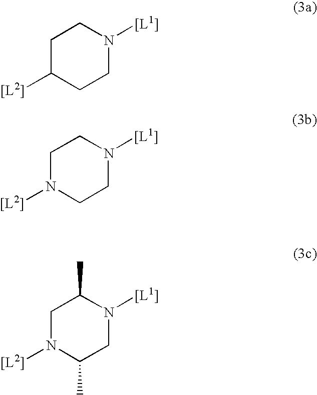

In the formulas (3a), (3b), and (3c), [L¹] and [L²] indicate the attachment points for the L¹ and L² groups, respectively, in formula (1). In these embodiments, L¹ is typically CO and L² is frequently CH₂ or CHMe. These formulas are presented to ensure clarity in describing certain preferred embodiments, and are not limitations on the scope of the invention.

Cy is a cyclic moiety, or it may be two cyclic moieties on a single atom of L². The cyclic moieties of Cy include aryl, heteroaryl, cycloaliphatic and cycloheteroaliphatic groups that can be optionally substituted. Cy may be, for instance, cyclohexyl, piperazinyl, benzimidazolyl, morpholinyl, pyridyl, pyrimidyl, phenyl, naphthyl and the like. Alternatively, Cy can represent a phenyl ring and a cyclohexyl ring both attached to one atom of the L² linker, or two phenyl rings attached in this fashion. Cy is preferably substituted or unsubstituted aryl or heteroaryl, and more preferably is an optionally substituted phenyl or two optionally substituted phenyls. In some preferred embodiments, Cy is a mono-substituted or disubstituted aryl group, preferably a phenyl group, and in others it is an unsubstituted phenyl or two unsubstituted phenyls. When Cy is a monosubstituted phenyl, the substituent is sometimes preferably at the para position. Para-fluoro phenyl is one preferred embodiment of Cy; unsubstituted phenyl is another such embodiment; and two phenyl rings is another.

Each cyclic moiety comprising Cy is optionally substituted with up to five substituents R⁶. Each substituent R⁶ on Cy is independently a hydrocarbyl residue (1-20C) containing 0-5 heteroatoms selected from O, S and N, or is an inorganic residue. Typically, R⁶ is one of the same groups set forth for R¹ above. Preferred R⁶ substituents include those selected from the group consisting of alkyl, alkenyl, alkynyl, aryl, arylalkyl, acyl, aroyl, heteroaryl, heteroalkyl, heteroalkenyl, heteroalkynyl, heteroalkylaryl, NH-aroyl, arylacyl, heteroarylacyl, halo, OR, NR₂, SR, SOR, SO₂R, OCOR, NRCOR, NRCONR₂, NRCOOR, OCONR₂, RCO, COOR, alkyl-COOR, alkyl-OOCR, SO₃R, CONR₂, SO₂NR₂, NRSO₂NR₂, CN, CF₃, and NO₂, wherein each R is independently H, alkyl, alkenyl or aryl or heteroforms thereof, and wherein two of said optional substituents on the same or adjacent atoms can be joined to form a fused, optionally substituted aromatic or nonaromatic, saturated or unsaturated ring which contains 3-8 members. More preferred embodiments of R⁶ include halo, alkyl (1-4C), and C1-4 alkyloxy, and more preferably, fluoro, chloro or methyl.

Cy may be substituted by up to five substituents; typically it is substituted with 0-3 or 1-2 substituents R⁶. In some preferred embodiments it is substituted once; in others it is unsubstituted. These R⁶ substituents may occupy any available positions of the ring of Cy. Typically, Cy comprises unsubstituted, mono-substituted, or disubstituted aryl or heteroaryl groups. In some preferred embodiments, Cy is disubstituted, and in more preferred embodiments it is unsubstituted or is mono-substituted. These substituents may themselves be optionally substituted with substituents similar to those listed above. Of course some substituents, such as halo, are not further substituted, as known to one skilled in the art. A particularly preferred embodiment of Cy is a para-halophenyl, especially para-fluorophenyl. Another preferred embodiment of Cy is two optionally substituted or especially two unsubstituted phenyl rings.

Two substituents on a ring comprising Cy can be joined to form a fused, optionally substituted aromatic or nonaromatic, saturated or unsaturated ring which contains 3-8 members and optionally contains 1-3 heteroatoms selected from N, O and S.

R³ represents a substituent suitable for an aryl ring as set forth above. Typically, R3 represents one of the groups set forth for R¹ above, other than hydrogen. Preferred embodiments include hydrocarbyl residues (1-6C) containing 0-2 heteroatoms selected from O, S and/or N and inorganic residues, including halo. R³ may be present ‘n’ times, where n is an integer of 0-2, preferably 0 or 1. Preferably, the substituents represented by R³ are independently halo, alkyl, heteroalkyl, OCOR, haloalkyl, OR, NRCOR, SR, or NR₂, wherein R is H, alkyl, aryl, or heteroforms thereof. More preferably R³ substituents are selected from C1-C8 alkyl, CF₃, C1-C8 alkoxy and halo, and most preferably methoxy, ethoxy, methyl, and chloro. Most preferably, n is 0 and the α ring is unsubstituted, except for L¹, or n is 1 and R³ is alkyl, halo or alkoxy, preferably chloro, methyl or methoxy. Often when R³ is present it is preferably at position 6 when L¹ is attached at position 5, or it is at position 5 when L¹ is attached at position 6, though R³ can also be at position 4 when Z⁴ is C, or at position 7 when Z⁵ is C. In some preferred embodiments, L¹ is at position 5, n is 0 or 1, and R³, if present, is at position 6. In such embodiments, R³ is typically methyl, methoxy, ethoxy, or chloro. If both Z⁴ and Z⁵ are N, n is 0 or 1, and typically in those embodiments, L¹ is attached at position 5 and R³, if present, must be at position 6.

Z³ is sometimes preferably NR⁷, but can also be S or O. Typical embodiments of R⁷ are the same as those described above for R¹. Preferred embodiments of R⁷ include H, optionally substituted alkyl, alkenyl, alkynyl, aryl, arylalkyl, acyl, arylacyl, aroyl, heteroaryl, heteroalkyl, heteroalkenyl, heteroalkynyl, heteroarylalkyl, heteroarylacyl, or R⁷ can be SOR, SO₂R, RCO, COOR, alkyl-COR, SO₃R, CONR₂, SO₂NR₂, CN, CF₃, NR₂, OR, alkyl-SR, alkyl-SOR, alkyl-SO₂R, alkyl-OCOR, alkyl-COOR, alkyl-CN, or alkyl-CONR₂, wherein each R is independently H, C1-C8 alkyl, C2-C8 alkenyl or C5-C10 aryl or heteroforms thereof. More preferably, R⁷ is hydrogen or is alkyl (1-4C), preferably methyl, or it is acyl (1-4C), or it is COOR wherein R is H, C1-C4 alkyl, C2-C4 alkenyl or C5-C10 aryl or hetero forms thereof. R⁷ is also sometimes preferably a substituted alkyl wherein the preferred substituents are ether groups or carbonyl- or sulfonyl-containing moieties. Other preferred R⁷ embodiments include hydroxyl-, amino-, and sulfhydryl-substituted alkyl substituents. Still other preferred embodiments include COR, COOR, and CONR₂ wherein R is defined as above.

As indicated in the foregoing descriptions, some of the preferred embodiments of the compounds of the invention include the following structural features:

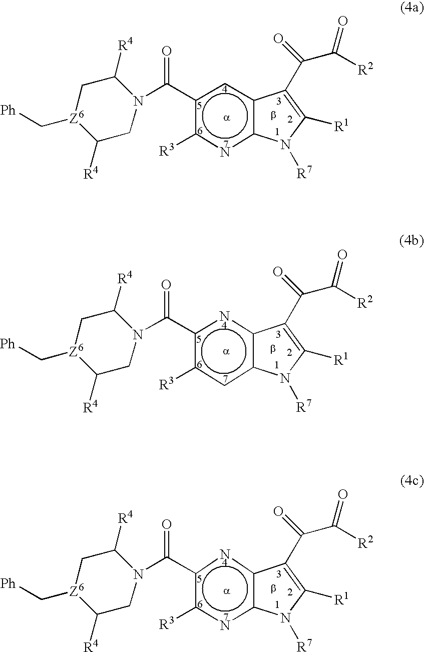

In formulas (4a), (4b), and (4c), Z⁶ represents N or CH; Ph represents an optionally substituted phenyl; R represents H or lower alkyl, especially methyl; and R³, R⁷, R¹, and R² are as defined for formula (1) except that R³ can alternatively be H in these formulas. In these embodiments, R¹ is typically H or methyl; R⁷ is typically H, methyl, or optionally substituted C1-C8 alkyl or C1-C8 heteroalkyl. R² can represent any of the groups described for R² above, but is often preferably NR₂, where each R is independently H or C1-C4 alkyl or C1-C4 heteroalkyl, and where the R groups in this NR₂ can optionally cyclize so that this NR₂ can be a 3-7 membered cyclic group such as a pyrrole, pyrroline, pyrrolidine, piperidine, piperazine, or morpholine, each of which is optionally substituted with halo, ═O, C1-C6 alkyl, C1-C6 heteroalkyl, or C1-C6 acyl or C1-C6 heteroacyl. Other substituents may optionally be present as well. These formulas are presented to enhance clarity in describing certain preferred embodiments, and are not limitations on the scope of the invention.

When the compounds of Formula 1 contain one or more chiral centers, the invention includes each optically pure form as well as mixtures of stereoisomers or enantiomers, including racemic mixtures. The azacyclic portion of these compounds in particular is often chiral, and single enantiomers are sometimes preferred while racemic mixtures or enantiomerically-enriched mixtures are also sometimes preferred.

The compounds of formula (1) may be supplied in the form of their pharmaceutically acceptable acid-addition salts including salts of inorganic acids such as hydrochloric, sulfuric, hydrobromic, or phosphoric acid and salts of organic acids such as acetic, tartaric, succinic, benzoic, salicylic, alkylsulfonic, or glucuronic acid and the like. Descriptions of suitable salts are provided in Stahl, P. Heinrich; Wermuth, Camille G. (Eds.), Handbook of Pharmaceutical Salts, 2002, Wiley-VCH, pp. 265-327. The azaindole rings in the compounds of formula (1) are weakly basic, so the ring itself can be protonated by strong acids such as the inorganic acids listed above as well as the stronger organic acids such as the alkyl- and arylsulfonic acids, trifluoroacetic acid, and the like. The protonated 7-azaindoles, for example, have a pKa of about 3-5, and can thus be protonated by acids having a pKa below about 4 or 5.

For those compounds where the azacyclic group has a relatively basic nitrogen, salts are more often formed on the azacyclic group instead of the aromatic bicyclic azaindole ring. Such acid addition salts readily form if the azacyclic group includes a nitrogen that is not acylated, as when the azacyclic group is piperazine and the L² linker is an alkylene: the protonated forms of those compounds have a pKa of about 6-9. Those compounds readily form salts from both organic and inorganic acids; the salts formed by addition of malonic acid, phosphoric acid, D- or L-tartaric acid, benzensulfonic acid, nitric acid, L-glutamic acid, D- or L-camphorsulfonic acid, sulfuric acid, hydrobromic acid, methanesulfonic acid, ethanesulfonic acid, p-toluenesulfonic acid, maleic acid, hydrochloric acid, as well as those acids mentioned above are specifically contemplated.

Furthermore, where the azacyclic group is relatively basic, it is possible to form di-salts by protonation of both the azacyclic and the azaindole moieties. Thus on treatment with a strong acid such as HCl, sulfuric acid, hydrobromic acid, nitric acid, phosphoric acid, trifluoroacetic acid, or an alkyl- or arylsulfonic acid, such compounds form di-salts. Upon ingestion, the compounds of the invention will form salts such as the hydrochloric acid addition salt or a di-hydrochloride salt, and these salt forms are specifically within the invention scope.

Where a carboxyl moiety is present on the compound of formula (1), the compound may also be supplied as a salt with a pharmaceutically acceptable cation such as lithium, sodium, potassium, or a substituted or unsubstituted ammonium species. Again, suitable counterions are described in Stahl, P. Heinrich; Wermuth, Camille G. (Eds.), Handbook of Pharmaceutical Salts, 2002, Wiley-VCH.

Many techniques for producing pro-drugs are well known in the art, and the invention also includes pro-drug forms of the compounds of formula (1). Thus where a compound of the invention has a carboxylic acid moiety, for example, the esters and readily-hydrolyzed amides of that carboxylic acid, such as amides formed by acylating the alpha-amine of an amino acid with that carboxylic acid are also within the scope of the invention. Similarly, where a compound of the invention has an amine group or a free hydroxyl group, the amides and esters formed with amino acids are examples of pro-drugs that are within the scope of the invention.

### SYNTHESIS OF THE INVENTION COMPOUNDS

The compounds of the invention may be synthesized by art-known methods, by methods described herein, or combinations of the two. The following reaction schemes are illustrative of approaches to synthesize particular embodiments. Those skilled in the art will appreciate that these approaches can be adapted to provide other compounds within the invention by using different available starting materials, or by making obvious changes to the sequence or order of reactions presented.

Scheme 1 illustrates a route used to synthesize compounds of the invention having Z⁵=N. 2,6-difluoropyridine can be converted to carboxylic acid A1 through treatment with a base such as lithium diisopropylamide at −78° C. in THF and then passing in a stream of dry CO₂. Carboxylic acid A1 can be converted to amide B1 through treatment with standard coupling reagents such as TBTU or EDCI and the appropriately substituted amine. B1 is dissolved in alcoholic solvent such as ethanol, methanol, or isopropanol whereupon ammonia gas is passed through the solution. The solution is sealed and heated until conversion to C1 is complete. Compound D1 is obtained by treating C1 with K-O^{t}Bu in the desired alcoholic solvent. Heating D1 in DMF with iodine and sodium periodate yields E1. Acetyl chloride was added to a solution of E1 in a solvent such as THF and a base such as pyridine, yielding F1. The trimethylsilylacetylene group was installed through treatment of F1 with trimethylsilyl acetylene in the presence of Pd(PPh₃)₂Cl₂, CuI, and an amine base. Cyclization to H1 is accomplished by refluxing a solution of G1 and tetrabutylammonium fluoride. At this point H1 can be functionalized by treatment with a base such as NaH, KOH, or LiHMDS followed by addition of an appropriate electrophile to give I1. I1 is then treated with oxalyl chloride in DCM, DCE, or chloroform. To the resulting intermediate is then added the desired nucleophile to give K1. H1 can be converted to J1 in a similar manner.

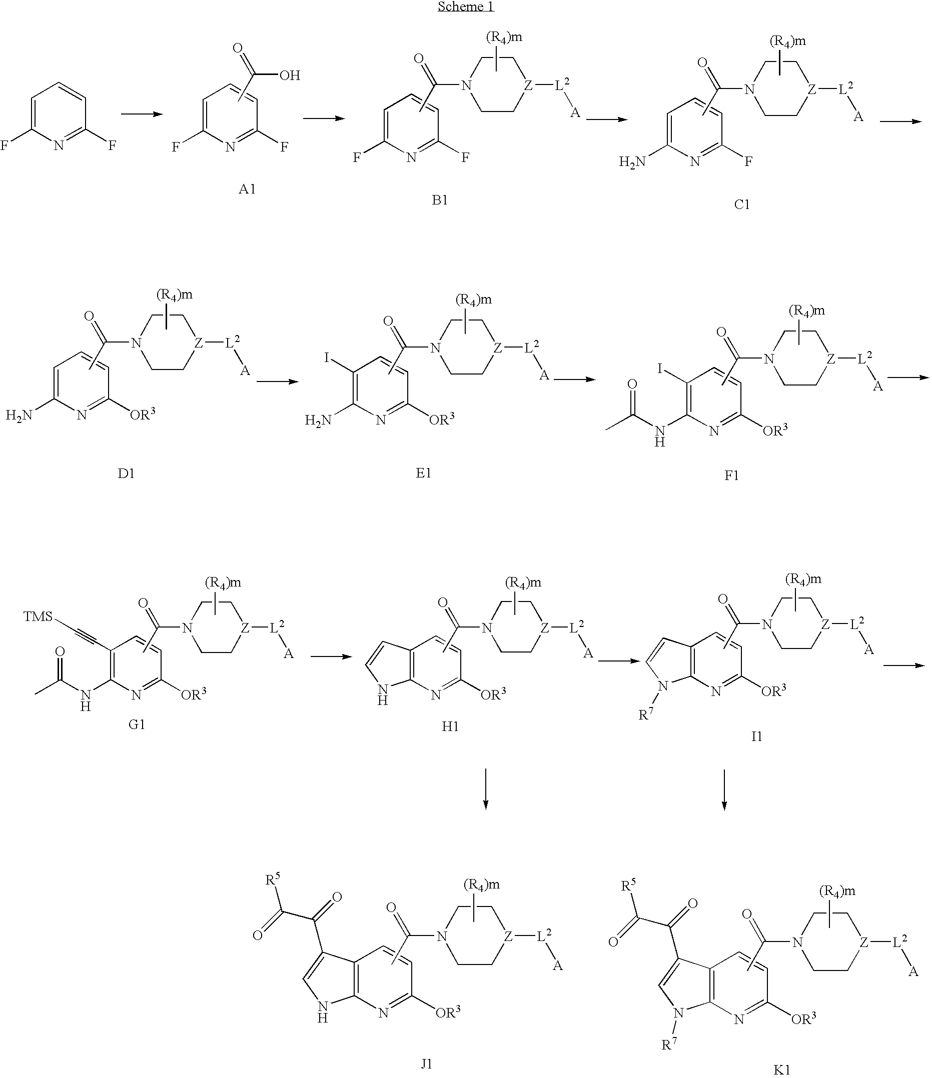

Alternatively, Scheme 2 illustrates how 2-substituted azaindoles can be prepared from D1, which is obtained as shown in Scheme 1, utilizing the method developed by Gassman (J. Amer. Chem. Soc., 96, 5495-5507 (1974)) wherein an appropriately substituted 2-aminopyridine is treated with N-chlorosuccinimide followed by the addition of a thiomethyl ketone and an amine base such as triethylamine to yield a 3-methylthio compound which is then reductively desulfurized using Raney nickel to provide L2. L2 can then be converted to M2 and O2 as described in Scheme 1 for converting H1 to J1 and H1 to K1.

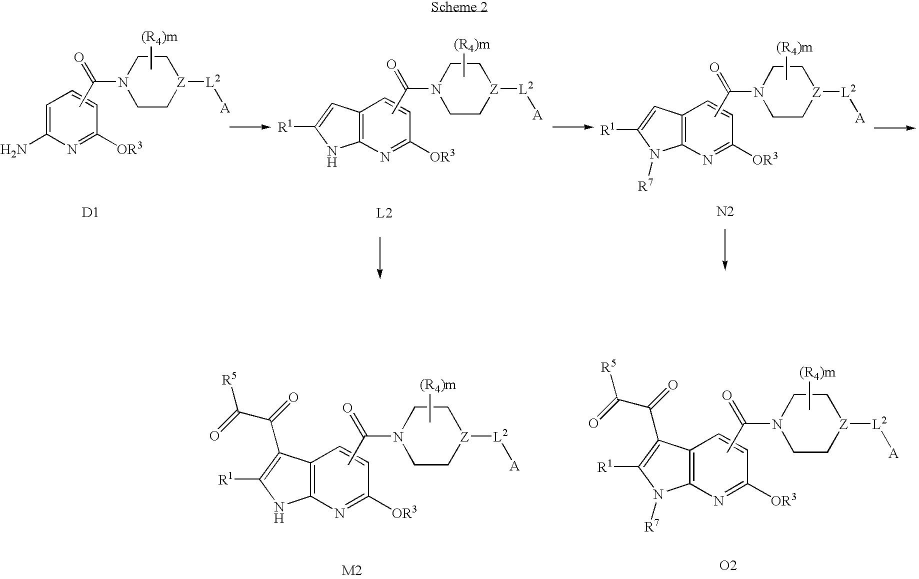

As shown in Scheme 3, the azaindole nitrogen of compounds within the invention can be aminated with an N-amination reagent, such as 2-nitro-4-(trifluoromethyl)phenyl hydroxylamine or 4-nitro-2-(trifluoromethyl)phenyl hydroxylamine, which are described in published patent application PCT/US2003/021888 (publication number WO2004/007462A1), and those described in Tetrahedron Lett., 23(37), 3835-3836 (1982), which are incorporated herein by reference. Compound A3 reacts with an N-amination reactant to give the N-substituted indole compound B3.

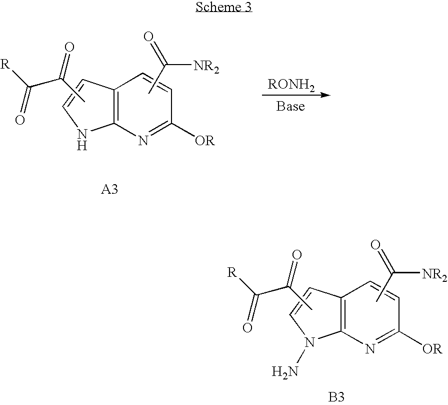

Examples of suitable N-aminating reagents are RONH₂ where R is an aromatic that is appropriately substituted with electron withdrawing groups such as one or two nitro groups; diarylphosphinyl; or a substituted sulfonyl group. Examples of these reagents include but are not limited to (Ar)ONH₂, (Ar₂PO)ONH₂, and (ROSO₂)ONH₂.

An alternate method to prepare 6-alkoxy-1H-pyrrolo[2,3-b]pyridine-5-carboxylic acid amides is provided in Scheme 4. Heating A4 in a solvent such as DMF with iodine and sodium periodate yields B4. This can be coupled with trimethylsilylacetylene in the presence of Pd(PPh₃)₂Cl₂, CuI, and an amine base to provide C4. Acetyl chloride is added to a solution of C4 in a solvent such as THF and in the presence of a base such as pyridine to yield D4. Cyclization is effected by heating D4 at reflux in the presence of tetrabutylammonium fluoride in a solvent such as THF, resulting in E4. E4 can be converted to its corresponding carboxylic acid F4 by treatment with aqueous base. Coupling of F4 with substituted amines under standard conditions using reagents like TBTU or EDCI results in compounds such as G4.

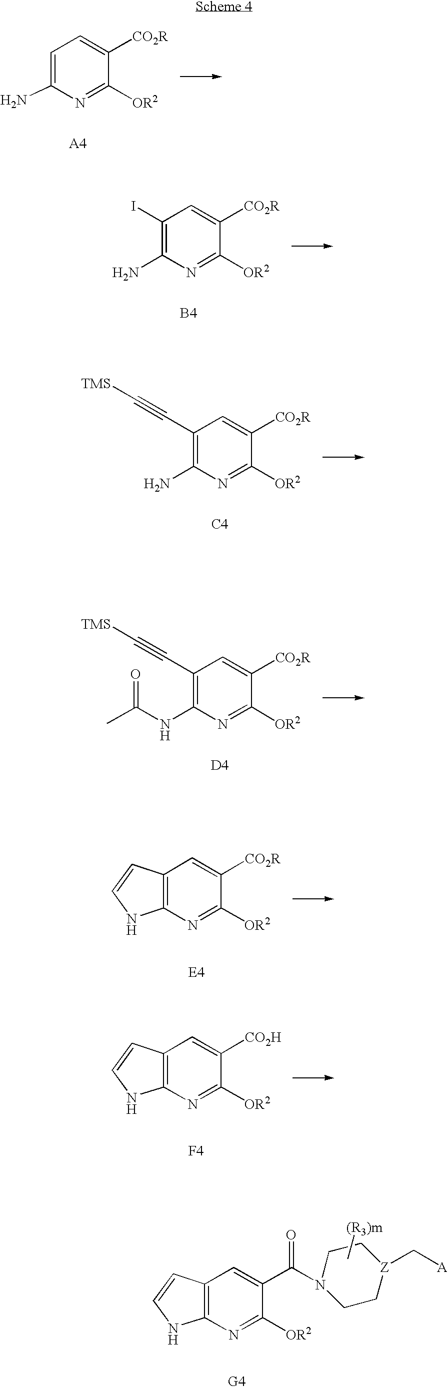

As Scheme 5 illustrates, various 6-amino-2-alkoxy-nicotinic acid esters can be prepared from 2,6-dichloronicotinic acid, which is first converted into ester A5 by heating in the appropriate alcohol with catalytic amounts of acid, such as hydrochloric or sulfuric acid. Compound B5 can be prepared by treating A5 with sodium alkoxides in a solvent such as dichloromethane or dichloroethane. By heating B5 and 4-methoxybenzylamine in the presence of an amine base in a polar aprotic solvent such as N-methylpyrrolidinone compound C5 is obtained. C5 is converted into D5 by heating in TFA until deprotection is complete.

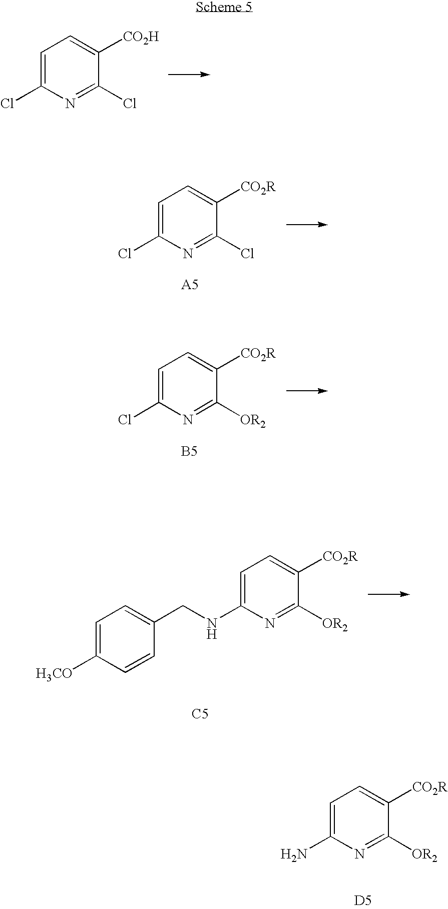

As depicted in Scheme 6, another method to make the requisite 6-amino-2-alkoxy nicotinic acid derivatives involves treatment of 2,6-dichloro-3-trifluromethylpyridine with dibenzylamine and an amine base in N-methylpyrrolidinone at elevated temperatures resulting in compound A6. Heating A6 and an appropriate sodium alkoxide in a solvent such as DMF yields compound B6. Removal of the two benzyl protecting groups can be achieved by treating a solution of compound B6 in wet methanol with palladium hydroxide on carbon under hydrogen pressure to give C6. C6 can be converted to D6 by heating it in methanol in the presence of sodium methoxide. The ester, E6, is obtained through treatment of D6 with dilute hydrochloric acid in the appropriate alcoholic solvent.

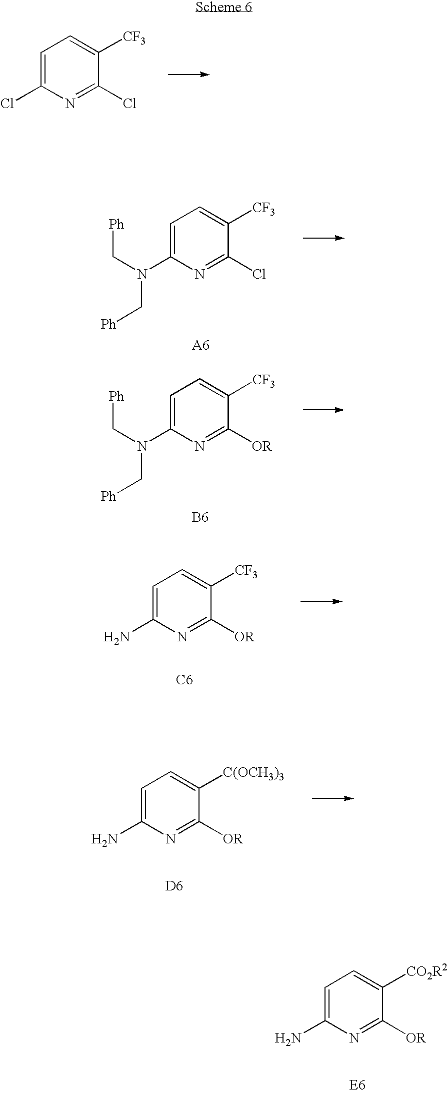

A method to prepare compounds having R³=alkyl, e.g. 6-alkyl-1H-pyrrolo[2,3-b]pyridine-5-carboxylic acid amides, is provided in Scheme 7. Compound A7 can be prepared by heating 5-bromo-6-alkyl-2-aminopyridines with Zn(CN)₂, 1,1-bis(diphenylphosphino)-ferrocene, and Pd₂(dba)₃ in a suitable solvent such as DMF. B7 is prepared by heating A7 in concentrated sulfuric acid. The carboxylic acid B7 can be converted to ester C7 by dissolving in the desired alcohol and treating with thionyl chloride. C7 can be converted to I7 in a manner similar to that described in Scheme 4 for converting A7 to G7. J7 can be prepared by treating I7 with a base such as NaH, KOH, or LiHMDS followed by addition of 2-(trimethylsilyl)ethoxymethyl chloride. J is then treated with oxalyl chloride in DCM, DCE, or chloroform. To the resulting intermediate is then added the desired nucleophile to give K7. Upon treatment with tetrabutylammonium fluoride, L7 is obtained. L7 can be converted to M7 by treating with a base such as NaH, KOH, or LiHMDS followed by the addition of the desired electrophile.

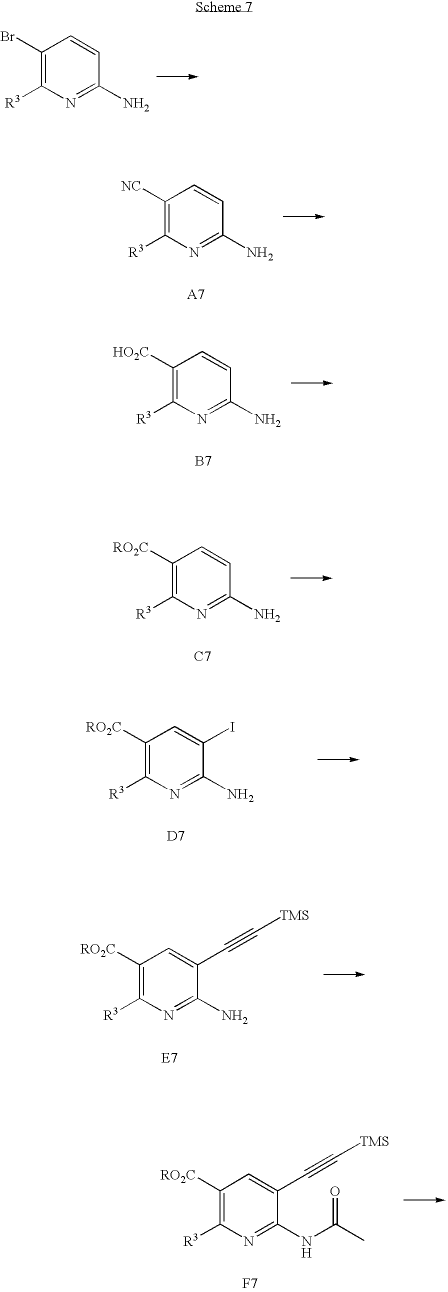

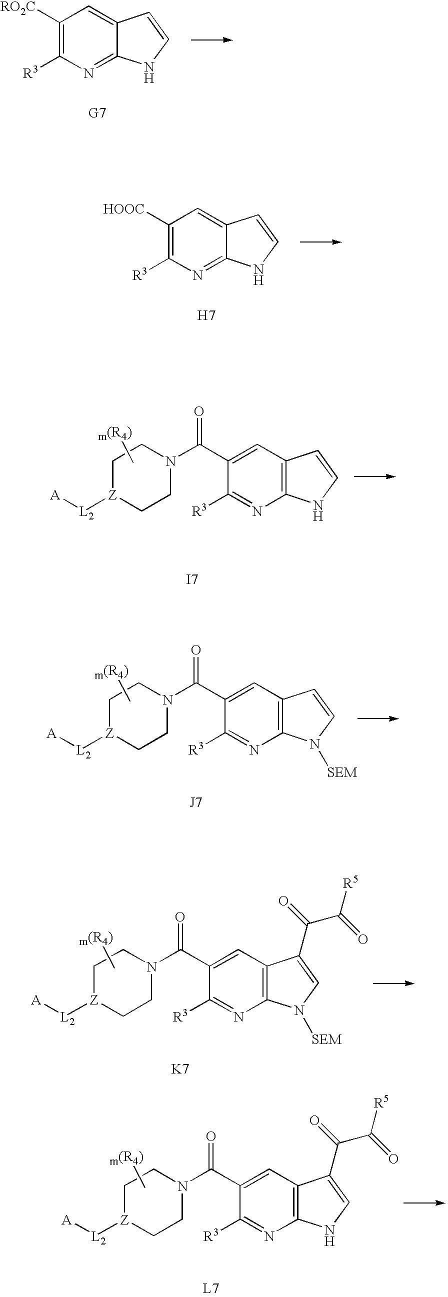

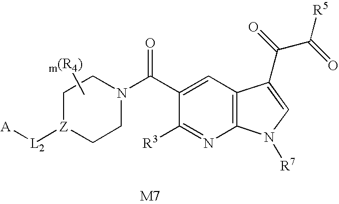

Schemes 8a and 8b depict a method used to make 4-azaindoles of the invention, compounds having Z⁴=N and Z⁵=C. This method uses a 3-amino pyridine and adds an acetylene group at position 2, which can then be cyclized to form the fused five-membered ring. 5-amino-2-cyanopyridine can thus be converted to 5-amino-2-nicotinic acid A8 by treating with sulfuric acid to provide the intermediate amide, which is then heated in water at 100° C. until conversion to carboxylic acid A8 is achieved. The ester B8 can be obtained by treating A8 with thionyl chloride in the desired alcoholic solvent. Synthesis of compound C8 can be achieved by heating B8 in a solvent such as DMF with iodine and sodium periodate. The trimethylsilylacetylene group can be installed through treatment of C8 with trimethylsilyl acetylene in the presence of Pd(PPh₃)₂Cl₂, CuI, and an amine base. Compound E8 can be obtained by adding acetyl chloride to D8 and pyridine in a solvent such as dichloromethane. Cyclization to F8 can be performed by refluxing a solution of E8 and tetrabutylammonium fluoride. F8 can be converted to its corresponding carboxylic acid by treatment with aqueous base. Carboxylic acid G8 can be converted to amide H8 through treatment with standard coupling reagents such as TBTU or EDCI and the appropriately substituted amine.

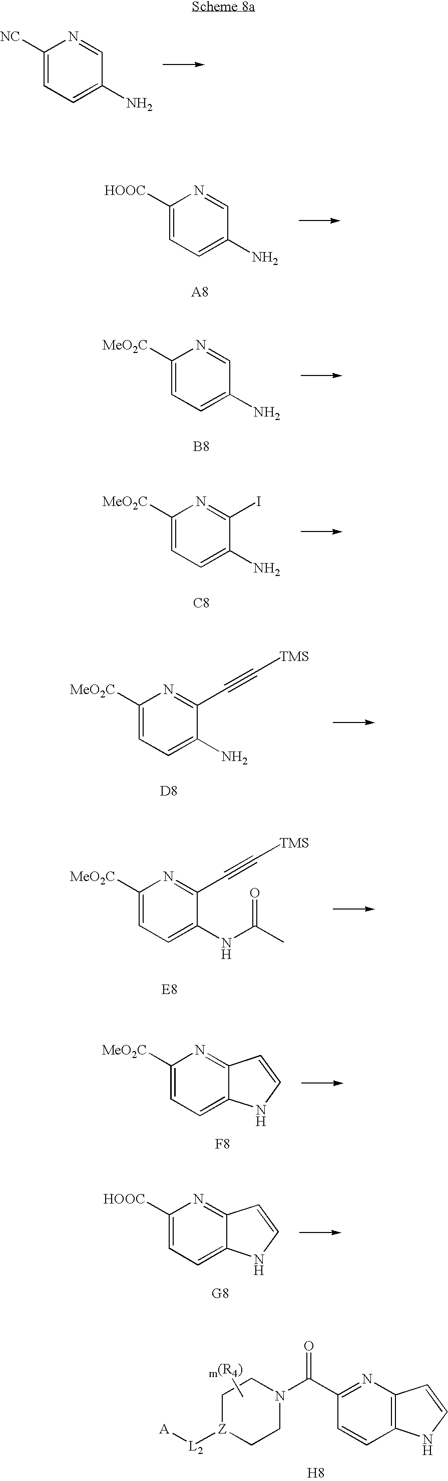

H8 is then treated with AlCl₃ and ethyl oxalyl chloride in a solvent such as dichloromethane to yield I8, which can be converted to its corresponding carboxylic acid K8 by treatment with aqueous base. Alternatively, I8 can be functionalized by treatment with base such as NaH, KOH, or LiHMDS followed by addition of an appropriate electrophile to give J8. J8 can then be converted to carboxylic acid L8 by treatment with aqueous base. K8 can then be converted to M8 using standard coupling reagents such as TBTU, EDCI, or DCC and the desired amine or alcohol. L8 can be converted to N8 in a similar manner.

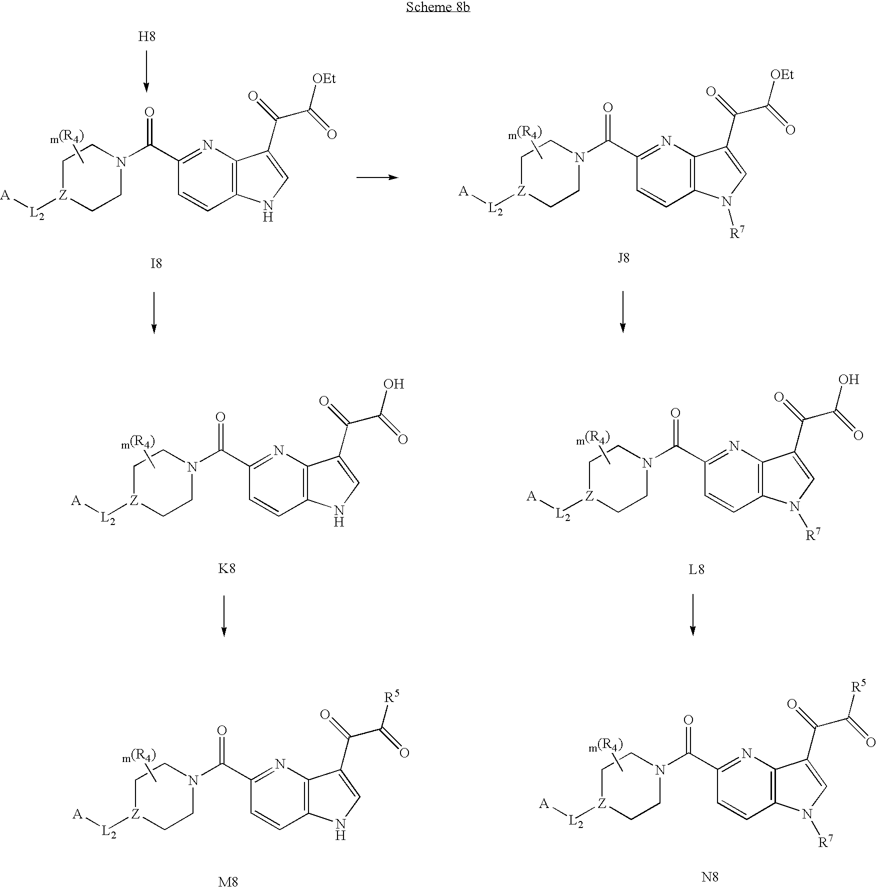

Scheme 9 illustrates the preparation of compounds within the scope of the invention having a halogen on the ring labeled α in formula (1). 5,6-dichloronicotinic acid is treated with diphenylphosphoryl azide and triethylamine in t-butanol to form A9. A9 is converted to B9 by heating with palladium acetate, 1,3-bis(diphenylphosphino)propane, triethylamine, and carbon monoxide. C9 is formed by treating B9 with trifluoroacetic acid in a solvent such as dichloromethane. C9 can be converted to I9 in a manner similar to that described in Scheme 4 for converting A4 to G4 and in Scheme 8 for converting B8 to H8. I9 can be converted to N9 and O9 in a manner similar to that described in Scheme 8 for converting H8 to M8 and N8.

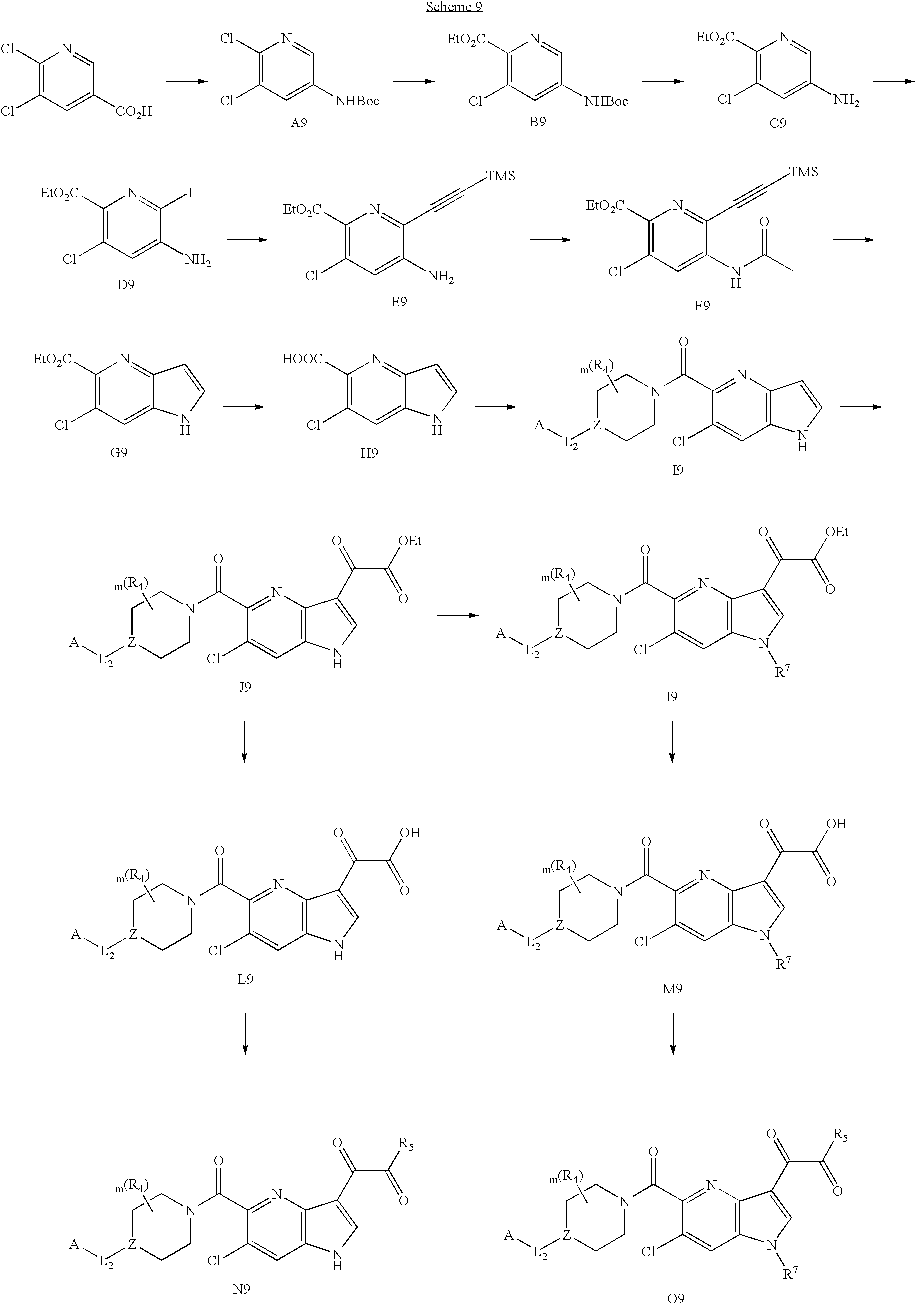

As shown in Scheme 10, compounds within the invention having an alkoxy group on the ring labeled α in formula (1) can be prepared from 2,3-dihydroxypyridine. The dihydroxypyridine is converted to A10 through treatment with NaOH and an alkylating agent such as dimethylsulfate. The intermediate formed is then treated with concentrated sulfuric and nitric acid to form the nitro compound. B10 can be prepared by heating A10 in a mixture of PCl₅ and POCl₃. The nitro compound can then be reduced with tin chloride in concentrated hydrochloric acid to yield C10. D10 is converted to J10 in a manner similar to that described in Scheme 4 for converting A4 to G4 and in Scheme 8 for converting B8 to H8. J10 can be converted to M10 or N10 as shown in Scheme 7 for converting I7 to L7 or M7.

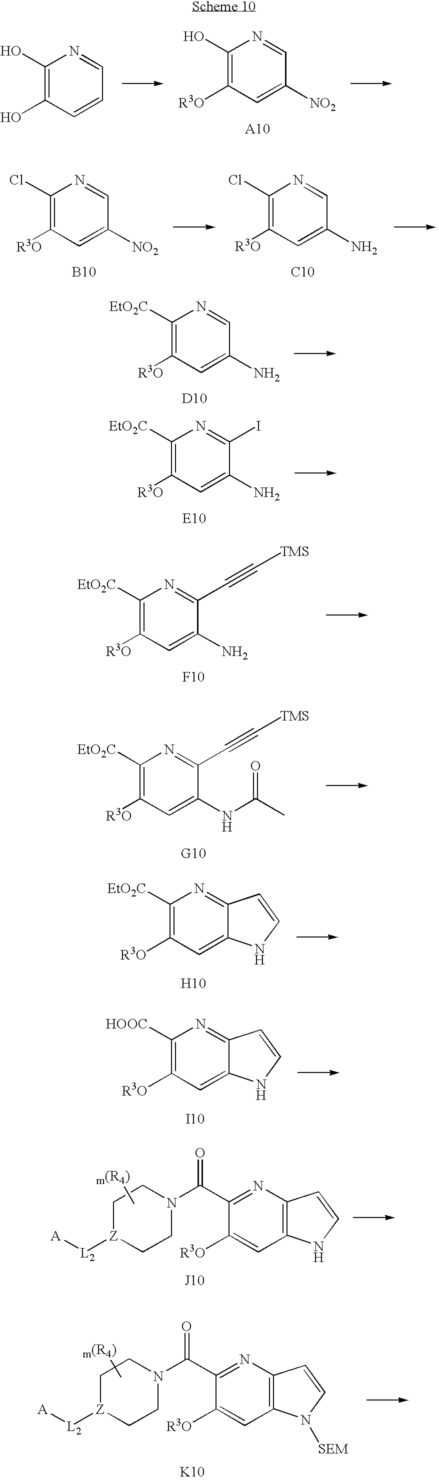

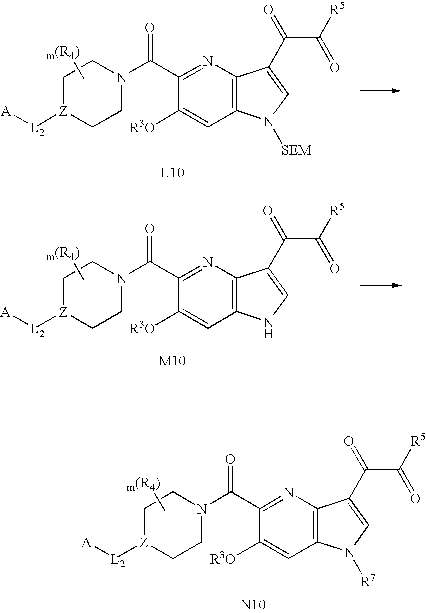

Scheme 11 depicts a method that can be used to prepare 4,7-diazaindoles of the invention, compounds having Z⁴=N and Z⁵=N. 2,6-Dichloropyrazine can be converted to A11 by heating with the appropriate alkoxide in the corresponding alcoholic solvent. Treatment of A11 with LDA in an appropriate anhydrous solvent such as THF followed by quenching with CO₂ gas results in B11. The ester C11 can be obtained by treating B11 with thionyl chloride in the desired alcoholic solvent. Heating C11 and 4-methoxybenzylamine in the presence of an amine base in a polar aprotic solvent such as N-methylpyrrolidinone (NMP) results in D11. D11 can be converted to E11 by heating in TFA until deprotection is complete. Synthesis of F11 can be achieved by heating E11 in a solvent such as DMF with iodine and sodium periodate. The trimethylsilylacetylene group can be installed through treatment of F11 with trimethylsilyl acetylene in the presence of Pd(PPh₃)₂Cl₂, CuI, and an amine base. Compound H11 can be obtained by adding acetyl chloride to G11 and pyridine in a solvent such as dichloromethane. Cyclization to I11 can be performed by refluxing a solution of H11 and tetrabutylammonium fluoride. I11 can be converted to its corresponding carboxylic acid by treatment with aqueous base. Carboxylic acid J11 can be converted to amide K11 through treatment with standard coupling reagents such as TBTU or EDCI and the appropriately substituted amine.

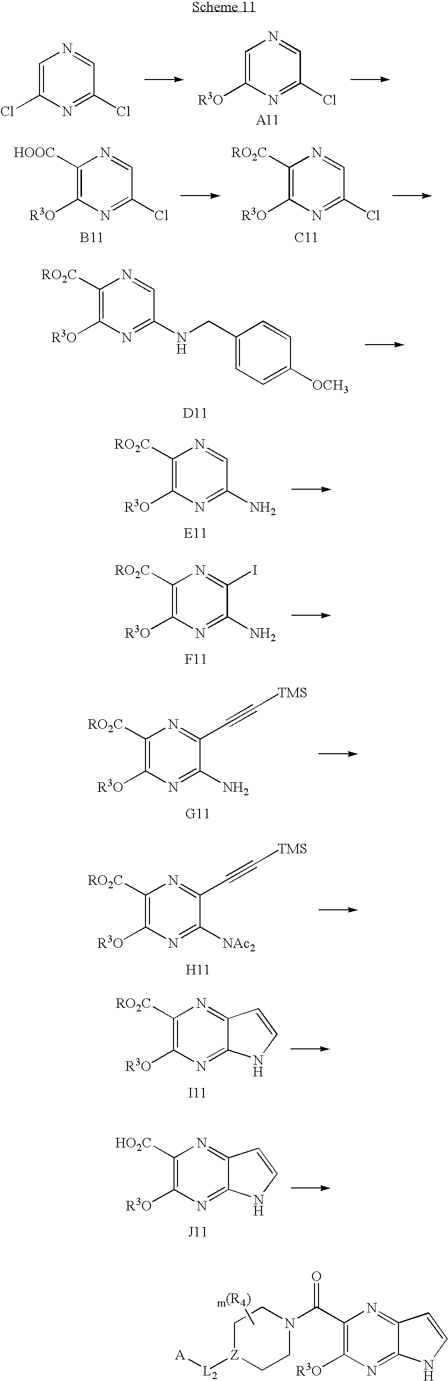

The pyrazines like K11 can be acylated and/or N-alkylated by the same procedures described herein for the compounds having either Z⁴=N or Z⁵=N.

Assays for p38 α Kinase Activity

Compounds of the invention may be tested for biological activity using the in vitro assay described below, or using methods described in the references cited herein.

Administration and Use

The compounds of the invention are useful among other indications in treating conditions associated with cytokine regulation and inflammation. Thus, the compounds of formula (1) or their pharmaceutically acceptable salts are used in the manufacture of a medicament for prophylactic or therapeutic treatment of mammals, including humans, in respect of conditions characterized by excessive production of cytokines and/or inappropriate or unregulated cytokine activity.

The compounds of the invention inhibit the production of cytokines such as TNF, IL-1, IL-6 and IL-8, cytokines that are important proinflammatory constituents in many different disease states and syndromes. Thus, inhibition of these cytokines has benefit in controlling and mitigating many diseases. The compounds of the invention are shown herein to inhibit a member of the MAP kinase family variously called p38 MAPK (or p38), CSBP, or SAPK-2. The activation of this protein has been shown to accompany exacerbation of the diseases in response to stress caused, for example, by treatment with lipopolysaccharides or cytokines such as TNF and IL-1. Inhibition of p38 activity, therefore, is predictive of the ability of a medicament to provide a beneficial effect in treating diseases such as Alzheimer's, coronary artery disease, congestive heart failure, cardiomyopathy, myocarditis, vasculitis, restenosis, such as occurs following coronary angioplasty, atherosclerosis, IBD, rheumatoid arthritis, rheumatoid spondylitis, osteoarthritis, gouty arthritis and other arthritic conditions, multiple sclerosis, acute respiratory distress syndrome (ARDS), asthma, chronic obstructive pulmonary disease (COPD), chronic pulmonary inflammatory disease, cystic fibrosis, silicosis, pulmonary sarcosis, sepsis, septic shock, endotoxic shock, Gram-negative sepsis, toxic shock syndrome, heart and brain failure (stroke) that are characterized by ischemia and reperfusion injury, surgical procedures, such as transplantation procedures and graft rejections, cardiopulmonary bypass, coronary artery bypass graft, CNS injuries, including open and closed head trauma, inflammatory eye conditions such as conjunctivitis and uveitis, acute renal failure, glomerulonephritis, inflammatory bowel diseases, such as Crohn's disease or ulcerative colitis, graft vs. host disease, bone-fracture healing, bone resorption diseases like osteoporosis, soft tissue damage, type II diabetes, pyresis, psoriasis, cachexia, viral diseases such as those caused by HIV, CMV, and Herpes, and cerebral malaria.

p38 MAP kinase plays a role in many biological processes. As mentioned above, it can act as a TNF mediator of the inflammatory process. In a related and preferred embodiment of the invention, the compounds of the invention can be used to treat disorders such as rheumatoid arthritis, myelodysplasia, and psoriasis. Also mentioned herein is the role that p38 has relative to cytokines such as IL-6 which have an effect on the proliferation of certain cell types. In a related and preferred embodiment of the invention, the compounds provided herein can be used to treat disorders such as Multiple myeloma, Hogkins and Non-Hodgkins lymphomas, renal carcinomas, and other cancers. P38 kinase also plays a role in certain structural and regenerative aspects associated with bone disorders, including but not limited to the regulation of osteoblast and osteoclast cell differentiation. In a related and preferred embodiment of the invention, the compounds provided herein can be used to treat such disorders as metastatic bone disease, osteolytic lesions, osteoarthritis, osteoporosis and improper bone healing. In yet another embodiment, inhibition of p38 kinase through administration of the compounds provided herein can be used to treat acute and chronic pain, including circumstances of neuropathy, diabetic or otherwise.

The manner of administration and formulation of the compounds useful in the invention and their related compounds will depend on the nature of the condition, the severity of the condition, the particular subject to be treated, and the judgment of the practitioner; formulation will depend on mode of administration. As the compounds of the invention are small molecules, they are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the particular compound(s) being used, the condition(s) being treated and the judgment of the practitioner.

It should be noted that the compounds of formula (1) can be administered as individual active ingredients, or as mixtures of several embodiments of this formula. In addition, the inhibitors of p38 kinase can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation or activation.

As implied above, although the compounds of the invention may be used in humans, they are also available for veterinary use in treating animal subjects.

### EXAMPLES

The following examples are intended to illustrate but not to limit the invention, and to illustrate the use of the above Reaction Schemes. Those of skill in the art will appreciate that various combinations of the reactions disclosed can be used, and various starting materials known in the art can be employed for the preparation of other compounds within the invention.

Compounds prepared and described herein are often characterized by high performance liquid chromatography (HPLC) using a mass spectrum detector (LC-MS). The LC provides information about the purity of the compound, and most new compounds were purified to at least about 95% purity by LC. The mass spectrum obtained generally included a molecular ion having the mass of the expected product plus one, corresponding to a protonated species, and is reported as M+H or equivalently as M+1 to indicate that the observed molecular ion corresponds to the molecular weight of the protonated species, as expected. The LC-MS data thus provides evidence that the compound having the structure shown was produced by the reaction, as expected. The HPLC retention time in minutes is reported for some compounds, often as Rf, and the HPLC conditions are then usually reported as Condition A or Condition B, which provides further characterization of the compounds.

HPLC Condition A uses a Phenomenex 30×4.6 mm column, model no. 00A-4097-EO. The flow rate is 2.00 mL/min, beginning with 95:5 ratio of water to acetonitrile, with each solvent containing 0.1% trifluoroacetic acid (TFA). The elution profile includes a linear gradient from a 95:5 water-acetonitrile ratio to a 5:95 ratio over the first 5 minutes, then 0.5 min at this ratio before returning to the 95:5 ratio.

HPLC Condition B uses a Merck AGA Chromolith Flash 25×4.6 mm column, model no. 1.51463.001. The flow rate is 3.00 mL/min, beginning with 95:5 ratio of water to acetonitrile, with each solvent containing 0.1% trifluoroacetic acid (TFA). The elution profile includes a linear gradient from a 95:5 water-acetonitrile ratio to a 5:95 ratio over the first 2.5 minutes, then 0.25 min at this ratio before returning to the 95:5 ratio.

### Example 1

### Preparation of 1-{5-[4-(4-Fluoro-benzyl)-piperidine-1-carbonyl]-6-methoxy-1H-pyrrolo[2,3-b]pyridin-3-yl}-2-pyrrolidin-1-yl-ethane-1,2-dione

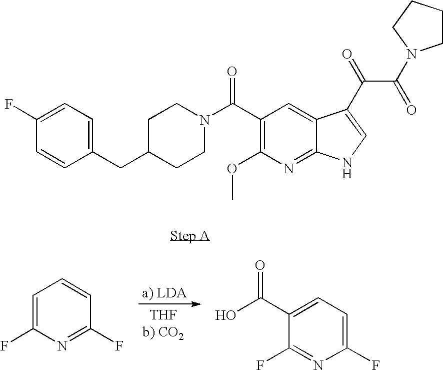

2,6-difluoropyridine-3-carboxylic acid (1) was prepared by using the method described by Rewcastle, G. W., et al., J. Med. Chem. (1996) 39:1823-1835.

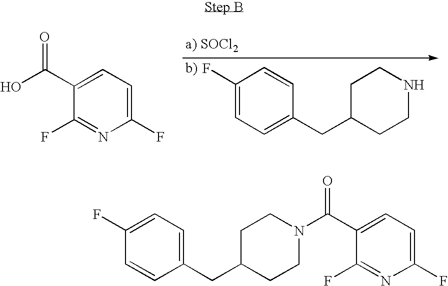

2,6-difluoropyridine-3-carboxylic acid (13.14 g) was suspended in dichloromethane (200 mL) and was cooled to 0° C. To this, under nitrogen atmosphere, was added thionyl chloride (30.14 mL, 413.2 mmol) dropwise. The ice-bath was removed and the mixture was refluxed for 3 h. The solvent was removed in vacuo. The product was taken up in dichloromethane (200 mL), stirred in an ice-bath and 4-fluorobenzylpiperidine hydrochloride (20.93 g, 91 mmol) was added followed by the dropwise addition of DIPEA (28.7 mL, 165.3 mmol). This was removed from the ice-bath and stirring continued for an additional 2 h at RT. The reaction mixture was poured into water and the organic layer was separated. The water layer was further extracted with dichloromethane (100 mL). The combined organic extracts was dried over sodium sulfate and evaporated. The residue was purified on a column of silica gel, eluting with ethyl acetate-hexane (20-50% ethyl acetate, gradient) to yield 23.58 g of the desired product.

LC-MS: 335, M+1

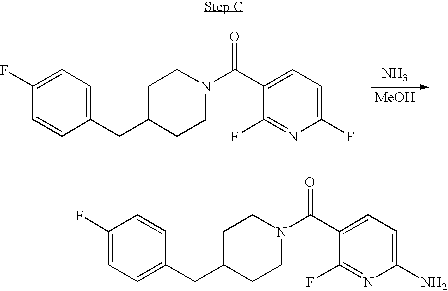

(2,6-Difluoro-pyridin-3-yl)-[4-(4-fluoro-benzyl)-piperidin-1-yl]-methanone (23.58 g) was dissolved in methanol (120 mL) in a sealed tube. This was cooled in a dry ice-acetone bath and a stream of ammonia gas was passed through the solution for about 5 min after which the reaction vessel was sealed. The mixture was heated in an oil bath at 60° C. for 20 h. The solvent was removed in vacuo and the residue was dissolved in dichloromethane and washed with water. The organic layer was dried over sodium sulfate and evaporated. The residue was purified on a column of silica gel eluting with ethyl acetate-hexane (50-70% ethyl acetate, gradient). The second major fraction contained the desired isomer (5.98 g, 25%).

LC-MS: 332, M+1

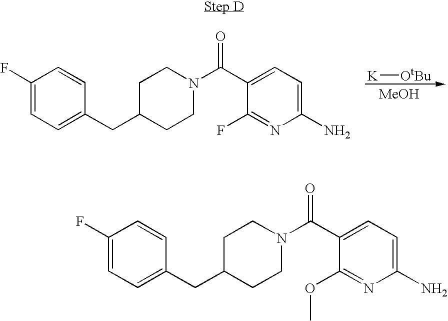

(6-Amino-2-fluoro-pyridin-3-yl)-[4-(4-fluoro-benzyl)-piperidin-1-yl]-methanone (5.86 g, 17.7 mmol) was taken in methanol (60 mL). Potassium-t-butoxide (9.9 g, 85.5 mmol) was added and the mixture was refluxed for 6 h. The methanol was removed under reduced pressure and the residue was extracted from water with ethyl acetate. After drying over sodium sulfate it was evaporated and the residue was purified on a column of silica gel with ethyl acetate-hexane (50-70%, gradient) as eluent to yield 5.46 g (90%) of the desired product.

LC-MS: 344, M+1

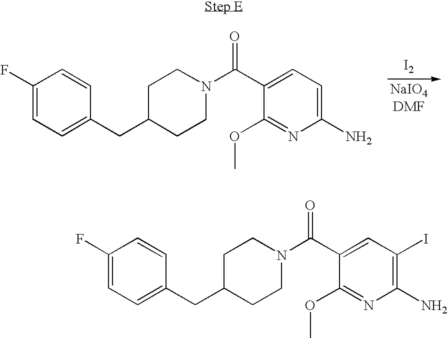

(6-Amino-2-methoxy-pyridin-3-yl)-[4-(4-fluoro-benzyl)-piperidin-1-yl]-methanone (5.44 g, 15.86 mmol) of was dissolved in dry DMF (70 mL). Iodine (3.23 g, 12.70 mmol, 0.8 eq.) was added followed by sodium periodate (1.36 g, 6.34 mmol, 0.4 eq.). The mixture was heated at 50° C. under nitrogen with stirring for 4.5 h. It was then poured into water and extracted with ethyl acetate (3×100 mL). The combined extract was washed with dilute sodium thiosulfate solution to remove the excess iodine. The ethyl acetate extract was dried over sodium sulfate and evaporated. The residue was purified on a column of silica gel eluting with ethyl acetate-hexane (20-40% ethyl acetate, gradient) to yield 6.46 g (86.8%) of the desired product.

LC-MS: 470, M+1

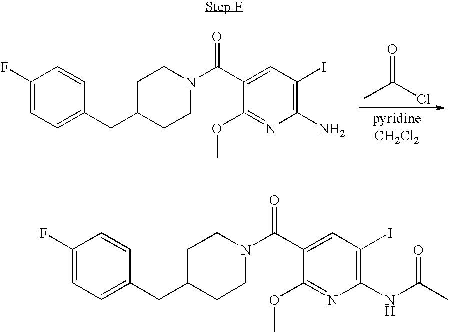

(6-Amino-5-iodo-2-methoxy-pyridin-3-yl)-[4-(4-fluoro-benzyl)-piperidin-1-yl]-methanone (6.46 g, 13.8 mmol) was taken in dry THF (100 mL). Pyridine (1.7 mL, 20.7 mmol) was added and the mixture was cooled in an ice-bath. To this mixture was added dropwise under nitrogen acetyl chloride (1.3 mL, 18 mmol) in dry THF (10 mL). After the addition, the ice-bath was removed and stirring continued at RT for another 20 h. The solvent was removed under reduced pressure and the residue was taken up in water and extracted with dichloromethane (3×100 mL). The combined extracts were dried over sodium sulfate and evaporated. The residue was purified on a column of silica gel eluting with ethyl acetate-hexane (40-70% ethyl acetate, gradient) to yield 4.91 g (69.6%) of the desired product.

LC-MS: 511.

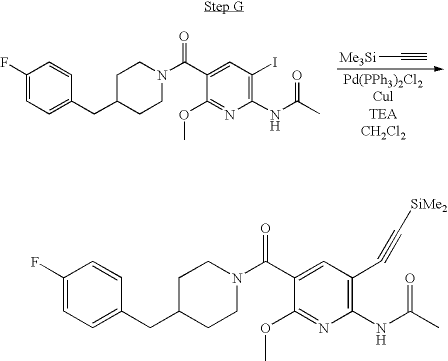

N-{5-[4-(4-Fluoro-benzyl)-piperidine-1-carbonyl]-3-iodo-6-methoxy-pyridin-2-yl}-acetamide (4.9 g, 9.59 mmol) was taken in dry dichloromethane (100 mL) and TEA (1.6 mL, 11.51 mmol) was added. The mixture was cooled in an ice-bath and Pd(PPh3)2Cl2 (35 mg, 0.05 mmol) and CuI (19 mg, 0.10 mmol) were added. To the stirred mixture was then added dropwise trimethylsilyl acetylene (1.49 mL, 10.55 mmol). The reaction mixture was removed from ice-bath and stirring continued for 20 h at RT. The reaction mixture was filtered to remove the solids and the filtrate was evaporated to dryness. The residue was purified on a column of silica gel eluting it with ethyl acetate-hexane (20-50% ethyl acetate, gradient), to yield 4.24 g (92%) of the desired compound.

LC-MS: 481.

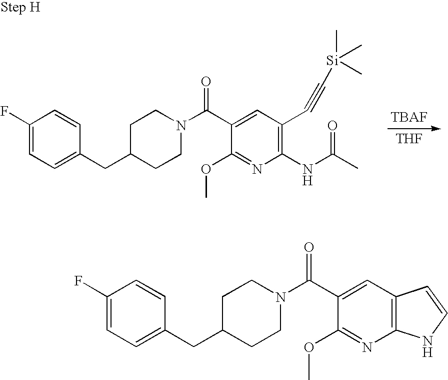

N-{5-[4-(4-Fluoro-benzyl)-piperidine-1-carbonyl]-6-methoxy-3-trimethylsilanylethynyl-pyridin-2-yl}-acetamide (4.24 g, 8.8 mmol) was dissolved in dry THF (50 mL). Tetrabutylammonium fluoride (1M solution in THF, 17.6 mL, 17.6 mmol) was added and the mixture refluxed with stirring for 3 h. The solvent was removed under reduced pressure and the residue was taken in water and extracted with dichloromethane (3×75 mL). The combined extracts were dried over sodium sulfate and evaporated. The residue was purified in a column of silica gel, eluting it with ethyl acetate-hexane (20-50% ethyl acetate, gradient) to yield 2.7 g of the desired product.

LC-MS: 368, M+1

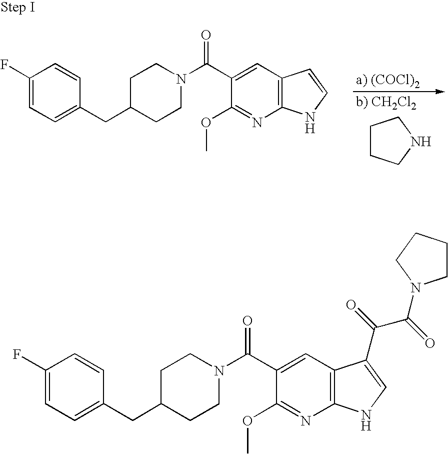

[4-(4-Fluoro-benzyl)-piperidin-1-yl]-(6-methoxy-1H-pyrrolo[2,3-b]pyridin-5-yl)-methanone (368 mg, 1 mmol) was dissolved in dry dichloromethane (5 mL). It was cooled in an ice-bath and oxalyl chloride (4.5 mL, 2 M solution in dichloromethane) was added. The mixture was stirred for 1 h at 0° C. and for another 4 h at room temperature. It was evaporated to dryness, redissolved in dichloromethane and treated with pyrrolidine (3 mmol). After stirring for 30 min, water was added and the product was extracted with dichloromethane (3×25 mL). The combined extracts were dried over sodium sulfate. After evaporation of the solvent, the product was purified via radial chromatography using chloroform-methanol (0-3% methanol) to yield 340 mg of the desired product.

M+H+493, Rf: 5.96 min, Condition A.

The following compounds were prepared by the same general method:

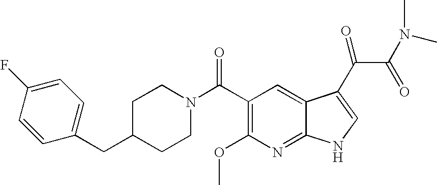

2-{5-[4-(4-Fluoro-benzyl)-piperidine-1-carbonyl]-6-methoxy-1H-pyrrolo[2,3-b]pyridin-3-yl}-N,N-dimethyl-2-oxo-acetamide: M+H⁺466.

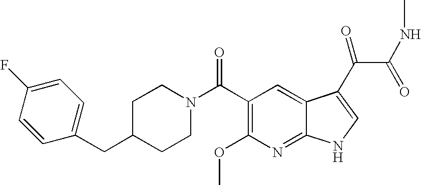

2-{5-[4-(4-Fluoro-benzyl)-piperidine-1-carbonyl]-6-methoxy-1H-pyrrolo[2,3-b]pyridin-3-yl}-N-methyl-2-oxo-acetamide: M+H⁺453, R_{f}: 3.347 min, Condition A.

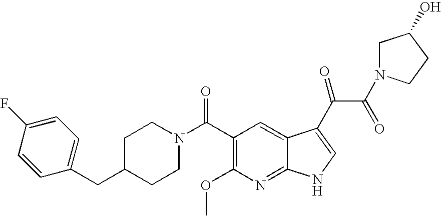

1-{5-[4-(4-Fluoro-benzyl)-piperidine-1-carbonyl]-6-methoxy-1H-pyrrolo[2,3-b]pyridin-3-yl}-2-(3-hydroxy-pyrrolidin-1-yl)-ethane-1,2-dione: M+H⁺509, R_{f}: 2.540 min, Condition A.

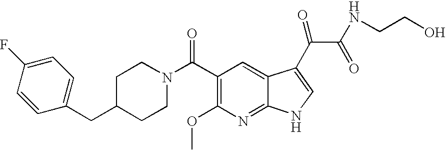

2-{5-[4-(4-Fluoro-benzyl)-piperidine-1-carbonyl]-6-methoxy-1H-pyrrolo[2,3-b]pyridin-3-yl}-N-(2-hydroxy-ethyl)-2-oxo-acetamide: M+H⁺483, R_{f}: 2.740 min, Condition A.

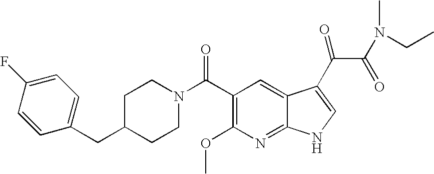

N-Ethyl-2-{5-[4-(4-fluoro-benzyl)-piperidine-1-carbonyl]-6-methoxy-1H-pyrrolo[2,3-b]pyridin-3-yl}-N-methyl-2-oxo-acetamide: M+H⁺481, R_{f}: 3.140 min, Condition A.

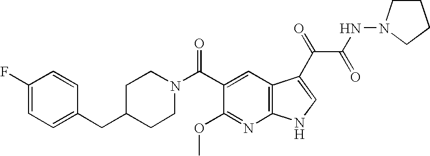

2-{5-[4-(4-Fluoro-benzyl)-piperidine-1-carbonyl]-6-methoxy-1H-pyrrolo[2,3-b]pyridin-3-yl}-2-oxo-N-pyrrolidin-1-yl-acetamide: M+H⁺508, R_{f}: 3.34 min, Condition A.

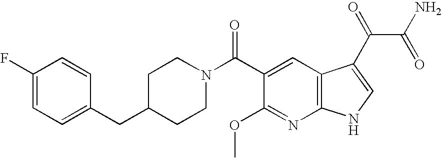

2-{5-[4-(4-Fluoro-benzyl)-piperidine-1-carbonyl]-6-methoxy-1H-pyrrolo[2,3-b]pyridin-3-yl}-2-oxo-acetamide: M+H⁺(439).

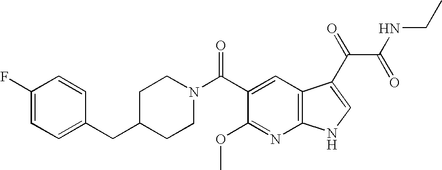

N-Ethyl-2-{5-[4-(4-fluoro-benzyl)-piperidine-1-carbonyl]-6-methoxy-1H-pyrrolo[2,3-b]pyridin-3-yl}-2-oxo-acetamide: M+H⁺467, R_{f}: 3.300 min, Condition A.

### Example 2

### Preparation of 1-{5-[4-(4-Fluoro-benzyl)-piperidine-1-carbonyl]-6-methoxy-1-methyl-1H-pyrrolo[2,3-b]pyridin-3-yl}-2-pyrrolidin-1-yl-ethane-1,2-dione

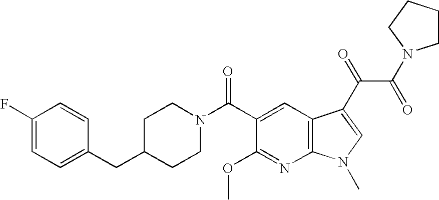

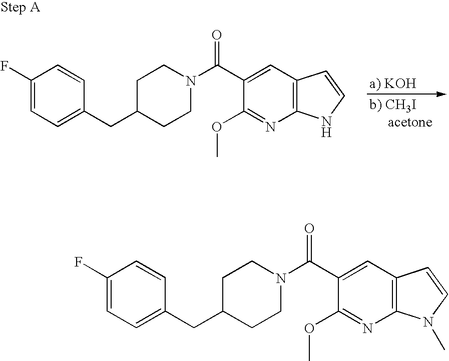

To a solution of [4-(4-Fluoro-benzyl)-piperidin-1-yl]-(6-methoxy-1H-pyrrolo[2,3-b]pyridin-5-yl)-methanone (100 mg, 0.27 mmol) and ground KOH (76 mg, 1.36 mmol) in anhydrous acetone (15 mL) was added iodomethane (96 mg, 0.675 mmol) at 0° C. The reaction mixture was warmed to RT slowly and stirred overnight. The solvent was removed, and the residue was treated with water and extracted with EtOAc. The combined organic layer was washed with brine, dried and concentrated. The residue was purified by chromatography on silica gel eluting with EtOAc:hexane (1:1) to give the desired product 100 mg (98% yield) as a white solid. M+H⁺(382).

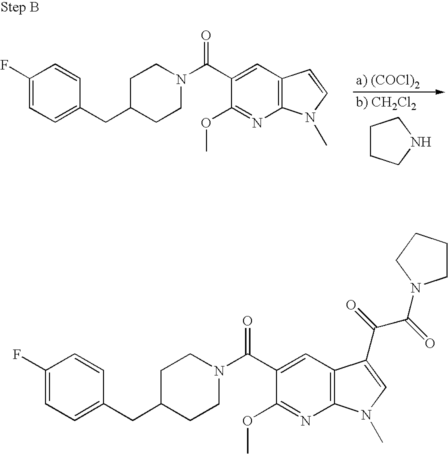

To a solution of [4-(4-Fluoro-benzyl)-piperidin-1-yl]-(6-methoxy-1-methyl-1H-pyrrolo[2,3-b]pyridin-5-yl)-methanone (100 mg, 0.26 mmol) in anhydrous CH₂Cl₂ (15 mL) was added oxalyl chloride (0.52 mL, 1.05 mmol, 2 M in CH₂Cl₂) at RT. The reaction mixture was stirred for 4 h. The reaction mixture was concentrated under reduced pressure. The residue was dried under vacuum for 1 h and dissolved in CH₂Cl₂ (15 mL). An excess amount of pyrolidine (74 mg, 1.04 mmol) was added to the reaction mixture. After stirring for 1 h, the reaction mixture was treated with water. The organic layer was separated and washed with brine, dried and concentrated. The residue was purified by chromatography on silica gel eluting with CH₂Cl₂: MeOH (95:5) to give the desired product (70 mg) in 53% yield as a white solid. M+H⁺507, R_{f}: 4.06 min, Condition A.

### Example 3

### Preparation of 1-{5-[4-(4-Fluoro-benzyl)-piperidine-1-carbonyl]-6-methoxy-1-methoxymethyl-1H-pyrrolo[2,3-b]pyridin-3-yl}-2-pyrrolidin-1-yl-ethane-1,2-dione

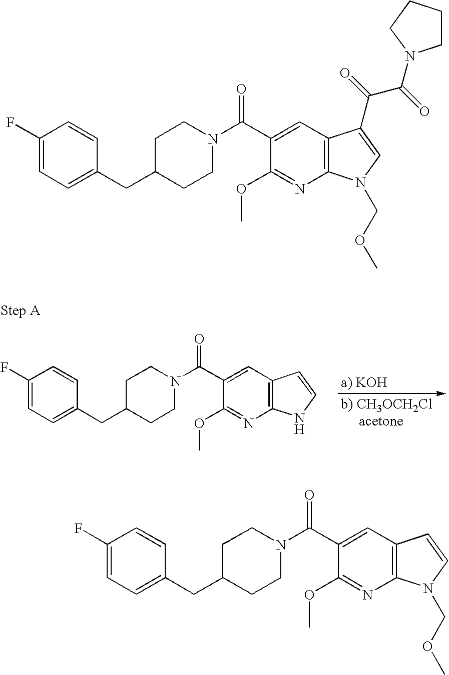

To a solution of [4-(4-Fluoro-benzyl)-piperidin-1-yl]-(6-methoxy-1H-pyrrolo[2,3-b]pyridin-5-yl)-methanone (150 mg, 0.41 mmol) and ground KOH (114 mg, 2 mmol) in anhydrous acetone (15 mL) was added MOM chloride (82 mg, 1 mmol) at 0° C. The reaction mixture was warmed to RT slowly and stirred overnight. The solvent was removed, and the residue was treated with water and extracted with EtOAc. The combined organic layer was washed with brine, dried and concentrated. The residue was purified by chromatography on silica gel eluting with EtOAc:hexane (1:1) to give the desired product 130 mg (77% yield) as a white solid. M+H⁺(411).

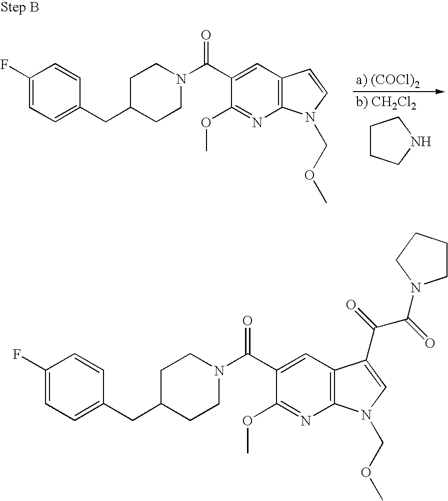

This compound was prepared according to the procedure in Example 1, Step I. M+H⁺538, R_{f}: 3.58 min, Condition A.

### Example 4

### Preparation of 1-{1-Amino-5-[4-(4-fluoro-benzyl)-piperidine-1-carbonyl]-6-methoxy-1H-pyrrolo[2,3-b]pyridin-3-yl}-2-pyrrolidin-1-yl-ethane-1,2-dione

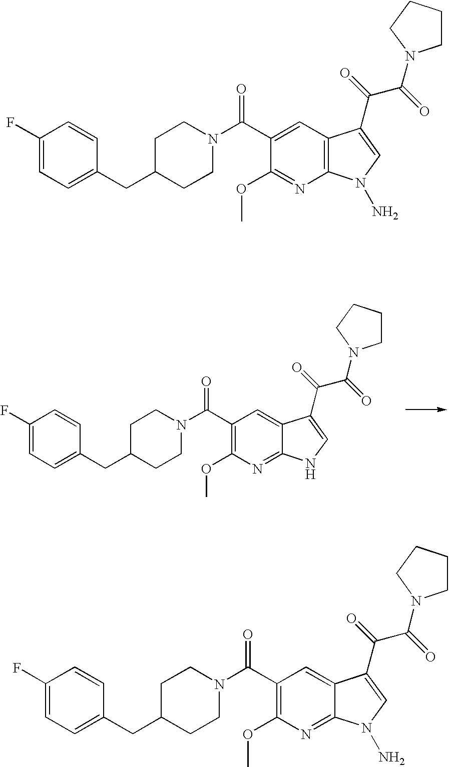

To a solution of 1-{5-[4-(4-Fluoro-benzyl)-piperidine-1-carbonyl]-6-methoxy-1H-pyrrolo[2,3-b]pyridin-3-yl}-2-pyrrolidin-1-yl-ethane-1,2-dione (76 mg, 0.154 mmol) in DMF (10 mL) was added 2,5-dinitrophenoxyamine (40 mg, 0.2 mmol) and K₂CO₃ (43 mg, 0.308 mmol). The reaction mixture was stirred at RT for 6 h, then treated with water (20 mL). The resulting mixture was extracted with EtOAc, washed with brine, dried (Na₂SO₄) and concentrated. The residue was purified by chromatography on silica gel eluting with 2% MeOH in CH₂Cl₂ to give 57 mg (73%) of the desired product as a white solid. M+H⁺508, R_{f}: 3.127 min, Condition A.

### Example 5

### Preparation of 1-{5-[4-(4-Fluoro-benzyl)-2R,5S-dimethyl-piperazine-1-carbonyl]-6-methoxy-1H-pyrrolo[2,3-b]pyridin-3-yl}-2-pyrrolidin-1-yl-ethane-1,2-dione

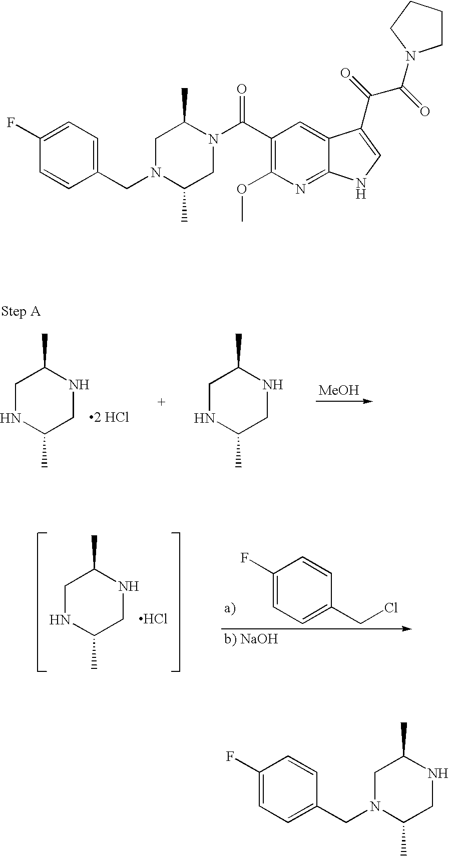

Trans-2,5-dimethylpiperazine (75.0 g) and trans-2,5-dimethylpiperazine dihydrochloride (122.83 g) are reacted together in equimolar quantities in methanol (370 mL) and the temperature raised to 68° C. and held for 30 min to generate two equivalents of trans-2,5-dimethylpiperazine monohydrochloride salt. This is treated with 1.005 molar equivalent of 4-fluorobenzyl chloride (100.4 g), added over 1 h with continued heating at reflux for 4 h, to give the monohydrochloride (1 equivalent) and trans-2,5-dimethylpiperazine dihydrochloride (1 equivalent). The mixture is cooled and the dihydrochloride trans-2,5-dimethylpiperazine salt is filtered off. The majority of the methanol is removed by distillation and heptane (300 mL) is added. The majority of the heptane was removed by distillation and then heptane (300 mL) was added again. This mixture was distilled until a temperature of 90° C. was reached. The mix was then cooled and water (390 mL) was added. The water layer is washed with heptane (2×300 mL) and the monohydrochloride trans-2,5-dimethylpiperazine-4-fluorobenzylpiperazine is treated with aqueous sodium hydroxide (24% w/w) at 0° C. to pH>13 to give the free base. To this is then added dichloromethane (3×300 mL) for extraction into the organic layer. The combined organics were washed with water (340 mL) and concentrated. The material is extracted into warm heptane and crystallized. (Yield: 108.6 g)

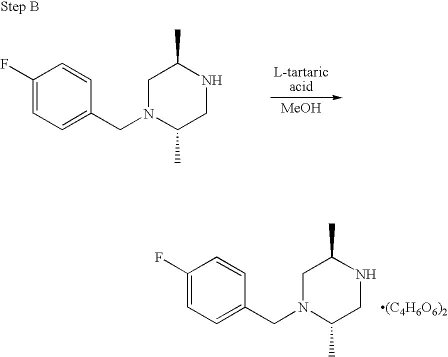

To a solution of (±) N-4-fluorobenzyl-trans-2,5-dimethylpiperazine (76.80 g) in methanol (750 mL) at 50° C. was added a solution of L-tartaric acid (77.01 g) in methanol (230 mL) over 30 min. The resulting solution was stirred at this temperature for 2 h then cooled to 42° C., at which time a small amount of seed crystal was added. This temperature was maintained for 30 min and was then cooled to 15° C. over ˜16 h. The resulting suspension was cooled to 5° C. and filtered. The filter cake was washed with cold methanol (230 mL) and further dried under vacuum. (Yield: 49%)

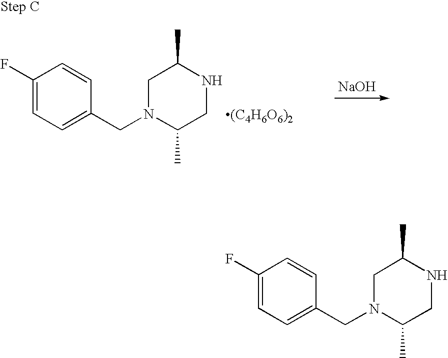

To a dichloromethane (370 mL) solution of the tartaric acid-piperazine salt (62.00 g) at RT was added a solution of NaOH (24.98 g) in water (140 mL) over 40 min. The resulting biphasic solution was stirred for 30 min and then allowed to separate. The dichloromethane solution was collected and the aqueous layer was further extracted with dichloromethane. The combined organics were washed with water and then concentrated to a volume of ˜150 mL. Heptane (250 mL) was then added and the mix was concentrated to a volume of ˜200 mL. This process was repeated and then the mixture was cooled to 0° C., held for 1 h, and then filtered and dried. (Yield: 19.48 g)

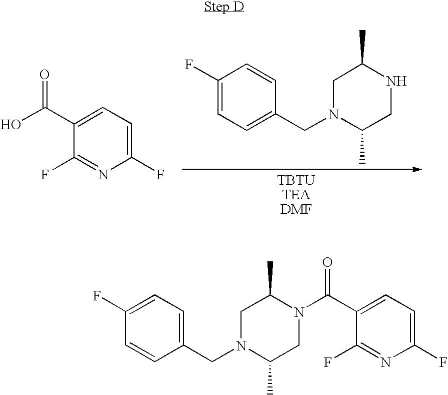

To a solution of 2,6-difluoropyridine-3-carboxylic acid (4.16 g, 26.2 mmol) and 1-(4-fluorobenzyl)-2S,5R-dimethyl piperazine (4.8 g, 21.8 mmol) in dimethylformamide was added TBTU (10.5 g, 32.7 mmol) followed by triethylamine (9 mL, 65.4 mmol). The reaction mixture was stirred overnight at RT and then poured into ice water. The precipitate formed was filtered and dissolved in dichloromethane. Two scoops of silica gel were added to the solution. The mixture was concentrate under reduced pressure. The residue was dry loaded on silica gel column eluting with EtOAc:hexane(4:6) to give 4 g (42%) of the desired product as a white solid. M+H⁺(364).

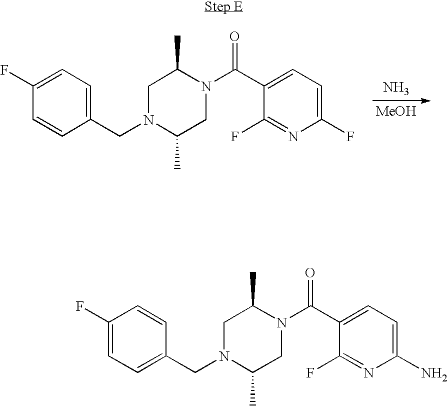

Ammonia gas (2 mL) was condensed and added to a cold solution of (2,6-Difluoro-pyridin-3-yl)-[4-(4-fluoro-benzyl)-2R,5S-dimethyl-piperazin-1-yl]-methanone (2 g, 8.26 mmol) in methanol (20 mL) in a Parr pressure reaction vessel at −78° C. The reaction vessel was sealed immediately and warmed to RT. The reaction mixture was heated at 60° C. overnight and cooled to −78° C. The reaction vessel was opened and the reaction mixture was then concentrated. The residue was purified by chromatography on silica gel eluting with EtOAc:hexane (1:1) then EtOAc:hexane(4:1) to give 750 mg (25%) of the undesired regioisomer, followed by 800 mg (27%) of the desired product as a white solid. M+H⁺(361).

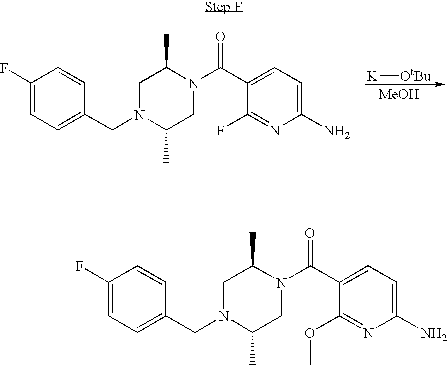

Potassium tert-butoxide (2.5 g, 22.2 mmol) was added to a solution of (6-Amino-2-fluoro-pyridin-3-yl)-[4-(4-fluoro-benzyl)-2R,5S-dimethyl-piperazin-1-yl]-methanone (1.6 g, 4.44 mmol) in anhydrous methanol (10 mL) at RT. The reaction mixture was refluxed overnight and then concentrated. The residue was treated with water and the resulting mixture was extracted with EtOAc. The combined organic extracts were washed with brine, dried and concentrated. The residue was purified by chromatography on silica gel eluting with EtOAc:hexane (2:1) to give 1.3 g (79%) of the desired product as a white foam. M+H⁺(361).

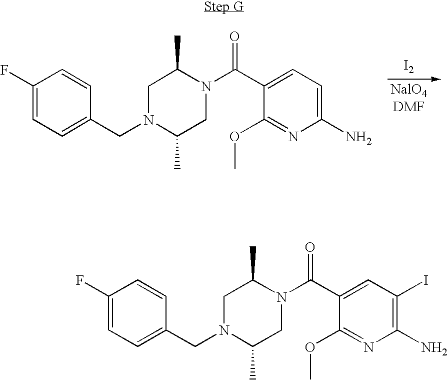

Method 1: To a solution of (6-Amino-2-methoxy-pyridin-3-yl)-[4-(4-fluoro-benzyl)-2R,5S-dimethyl-piperazin-1-yl]-methanone (800 mg, 2.15 mmol) in anhydrous DMF (20 mL) was added iodine (557 mg, 2.15 mmol) and sodium periodate (238 mg, 1.11 mmol). The reaction mixture was heated up at 50° C. overnight. The reaction mixture was poured into ice water and a solution of sodium thiosulfate (10%) was added to destroy the excess iodine. The precipitate formed was filtered. The crude product was purified by chromatography on silica gel (pre-treated with Et₃N) eluting with EtOAc:hexane (1:2) to give 522 mg (52%) of the desired product as a white foam. M+H⁺(499).

Method 2: Benzyltrimethylammonium dichloroiodate (1.46 g, 4.2 mmol) was added to a solution of (6-Amino-2-methoxy-pyridin-3-yl)-[4-(4-fluoro-benzyl)-2R,5S-dimethyl-piperazin-1-yl]-methanone (1.2 g, 3.2 mmol) and calcium carbonate (1.1 g, 11 mmol) in anhydrous dichloromethane (25 mL) at RT. The reaction mixture was stirred overnight. The organic layer was washed with water and 10% sodium thiosulfate, dried and concentrated. The residue was purified by chromatography on silica gel eluting with EtOAc:hexane (1:2) to give 910 mg (57%) of the desired product as a white foam. M+H⁺(499).

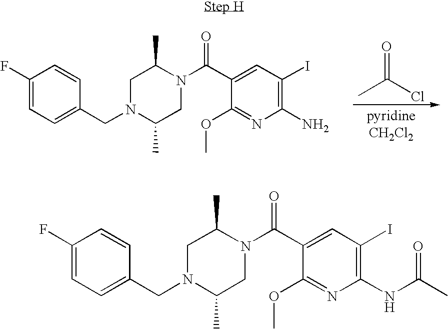

Acetyl chloride (48 mg, 0.62 mmol) was added to a solution of (6-Amino-5-iodo-2-methoxy-pyridin-3-yl)-[4-(4-fluoro-benzyl)-2R,5 S-dimethyl-piperazin-1-yl]-methanone (235 mg, 0.47 mmol) and pyridine (0.057 mL, 0.7 mmol) in dichloromethane (10 mL) at RT. The reaction mixture was stirred at RT overnight, and then treated with water. The organic layer was separated, dried and concentrated. The residue was purified by chromatography on silica gel eluting with EtOAc:hexane (1:1) to give 150 mg (59%) of the desired product as a colorless oil. M+H⁺(541).

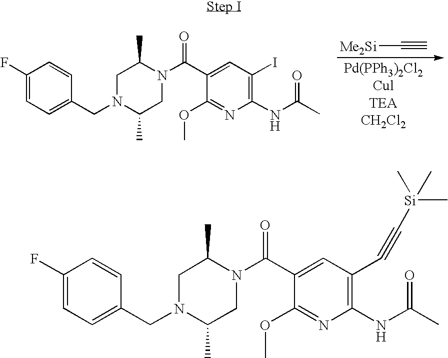

To a solution of N-{5-[4-(4-Fluoro-benzyl)-2R,5-dimethyl-piperazine-1-carbonyl]-3-iodo-6-methoxy-pyridin-2-yl}-acetamide (100 mg, 0.185 mmol), palladium bis(triphenylphosphine) dichloride (65 mg, 0.09 mmol), copper iodide (2 mg, 0.01 mmol) in anhydrous dichloromethane (5 mL) was added trimethlsilyacetylene (18 mg, 0.185 mmol) dropwise at 0° C. The reaction mixture was stirred at RT overnight, filtered through a plug of Celite® and concentrated. The residue was taken up into ethyl acetate, and washed with water and brine, dried and concentrated. The residue was purified by chromatography on silica gel eluting with EtOAc:hexane (1:1) to give 90 mg (96%) of the desired product. M+H⁺(511).

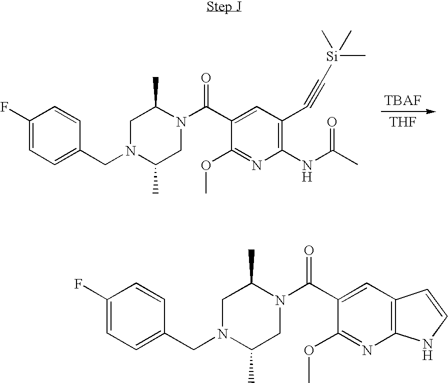

A mixture of N-{5-[4-(4-Fluoro-benzyl)-2R,5S-dimethyl-piperazine-1-carbonyl]-6-methoxy-3-trimethylsilanylethynyl-pyridin-2-yl}-acetamide (350 mg, 0.686 mmol) and tetrabutylammonium fluoride (1.37 mL, 1.37 mmol, 1.0 M in THF) in anhydrous THF was heated at reflux for 4 h. The reaction mixture was concentrated and the residue was taken up into ethyl acetate. The organic layer was washed with water, brine, dried and concentrated. The residue was purified by chromatography on silica gel eluting with EtOAc:hexane (4:6) to give 170 mg (63%) of the desired product as a colorless oil. M+H⁺(397).

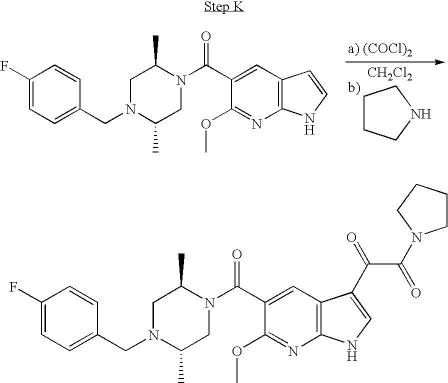

To a solution of [4-(4-Fluoro-benzyl)-2R,5S-dimethyl-piperazin-1-yl]-(6-methoxy-1H-pyrrolo[2,3-b]pyridin-5-yl)-methanone (170 mg, 0.43 mmol) in anhydrous CH₂Cl₂ (15 mL) was added oxalyl chloride (0.86 mL, 1.72 mmol, 2 M in CH₂Cl₂) at RT. The reaction mixture was stirred overnight. The reaction mixture was concentrated under reduced pressure. The residue was dried under vacuum for 1 h and dissolved in CH₂Cl₂. An excess amount of pyrrolidine (122 mg, 1.72 mmol) was added to the reaction mixture. After stirring for 1 h, the reaction mixture was treated with water. The organic layer was separated and washed with brine, dried and concentrated. The residue was purified by chromatography on silica gel eluting with CH₂Cl₂: MeOH (95:5) to give the desired product (150 mg) in 67% yield as a white solid. M+H⁺522, R_{f}: 2.167 min, Condition A.

The following compounds were prepared by the same basic approach.

### 2-{5-[4-(4-Fluoro-benzyl)-2R,5S-dimethyl-piperazine-1-carbonyl]-6-methoxy-1H-pyrrolo[2,3-b]pyridin-3-yl}-2-oxo-acetamide

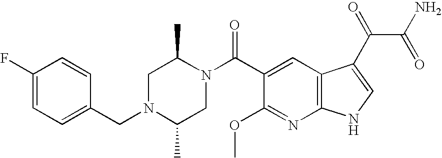

M+H⁺468, R_{f}: 1.76 min, Condition A.

### 2-{5-[4-(4-Fluoro-benzyl)-2R,5S-dimethyl-piperazine-1-carbonyl]-6-methoxy-1H-pyrrolo[2,3-b]pyridin-3-yl}-N,N-dimethyl-2-oxo-acetamide

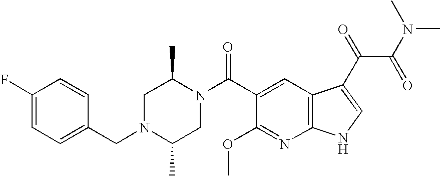

M+H+496, Rf: 1.833 min, Condition A.

### N-Ethyl-2-{5-[4-(4-fluoro-benzyl)-2R,5S-dimethyl-piperazine-1-carbonyl]-6-methoxy-1H-pyrrolo[2,3-b]pyridin-3-yl}-N-methyl-2-oxo-acetamide

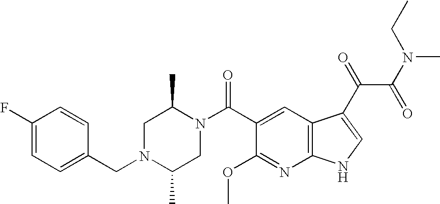

M+H⁺510, R_{f}: 1.94 min, Condition A.

### 1-{5-[4-(4-Fluoro-benzyl)-2R,5S-dimethyl-piperazine-1-carbonyl]-6-methoxy-1H-pyrrolo[2,3-b]pyridin-3-yl}-2-(3-hydroxy-pyrrolidin-1-yl)-ethane-1,2-dione

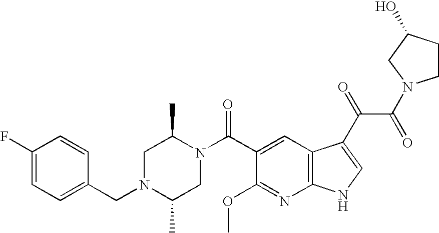

M+H⁺538, R_{f}: 1.68 min, Condition A.

### Example 6

### Preparation of 2-(5-{4-[1R-(4-Fluoro-phenyl)-ethyl]-2R-methyl-piperazine-1-carbonyl}-6-methoxy-1H-pyrrolo[2,3-b]pyridin-3-yl)-N-methyl-2-oxo-acetamide

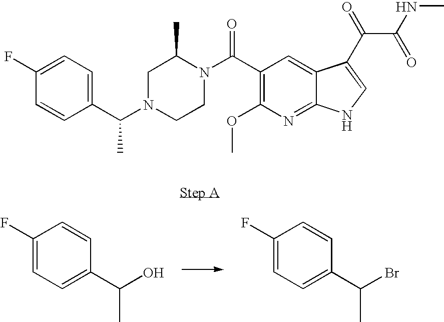

4-Fluoro-α-methylbenzyl alcohol (7 g, 6.3 ml, 50 mmol) was added to 50 mL 48% aqueous solution of HBr at 0° C. The solution was allowed to stir 3 h at RT, at which time it was extracted with hexane. After drying and concentration, 10 g of a colorless oil was obtained. M+H⁺(203).

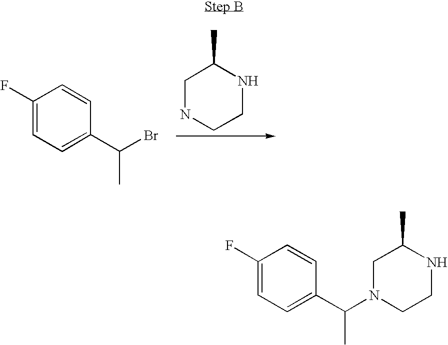

To 6.4 g 1-(4-fluorophenyl)-ethyl bromine in 100 mL DMF was added the 2R-methylpiperazine. The mixture was then stirred overnight at room temperature. The solution was evaporated and the residue was then filtrated on a small quantity of silica gel, washing with ethyl acetate and methanol. Purification was carried out using flash chromatography, CHCl₃/MeOH/Et₃N=90/8/2 (or AcOEt/MeOH=90/10). 6.2 g (90%) of pure compound (mixture inseparable of two diasteromers) was obtained. M+H⁺(223).

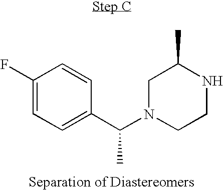

To the mixture of two diastereomers of 1RS-[1-(4-fluoro-phenyl)-ethyl]-3R-methyl-piperazine (1 g, 4.5 mmol) in methanol (2.5 mL), was added a solution of L-tartaric acid (1.4 g, 9 mmol) in methanol (4.2 mL). Crystallization is effected by keeping the resulting mixture at 0° C. over 30 h. The resulting material was filtered and then 15% NaOH was added to the mother liquid. The free base was extracted with ethyl acetate. Upon concentrated the resulting colorless oil was recrystallized in hexane two or three times until the desired purity is obtained (determined by proton NMR). M+H⁺(223).

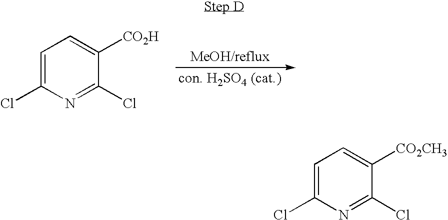

To a solution of 2,6-dichloronicotinic acid (30 g, 0.16 mol) in 150 mL methanol was added 3 mL of con. H₂SO₄ and the mixture was refluxed for 12 h. The methanol was evaporated off and the residue was dissolved in ethyl acetate, washed with water, 10% sodium carbonate solution, brine, dried with sodium sulfate and evaporated to yield the desired product (29.0 g, 87%) as white solid.

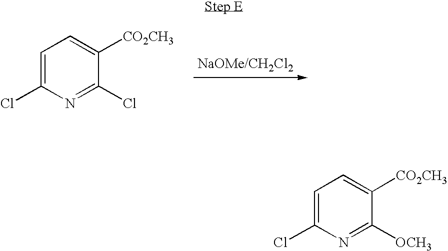

To a solution of 2,6-dichloro-nicotinic acid methyl ester (20.0 g, 0.1 mol) in dichloromethane (80 ml) at 0° C. was added NaOMe (8.1 g, 0.15 mol) slowly and stirred at 0° C. for 3 h. The reaction mixture was diluted with water, the organic layer was dried with sodium sulfate and evaporated to give an oily product which slowly solidified into a white solid (14.0 g, 70%).

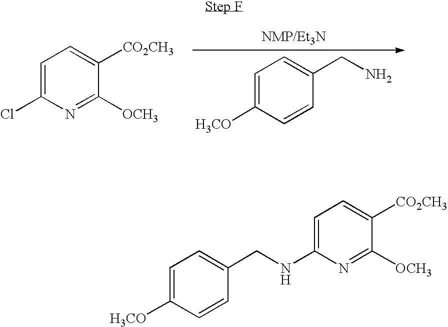

To a solution of 6-Chloro-2-methoxy-nicotinic acid methyl ester (18.5.0 g, 0.092 mol) in 50 mL of NMP was added p-methoxybenzylamine (19.0 g, 0.14 mol) and triethylamine (10.0 g, 0.1 mol). The mixture was heated to 70° C. for 4 h, cooled and diluted with water and extracted with ethyl acetate, washed with water, brine, dried with sodium sulfate, and evaporated to get an oil which was precipitated with ethylacetate/hexane mixture (1:1). This was then filtered and dried to yield 15 g (60%) of the target compound.

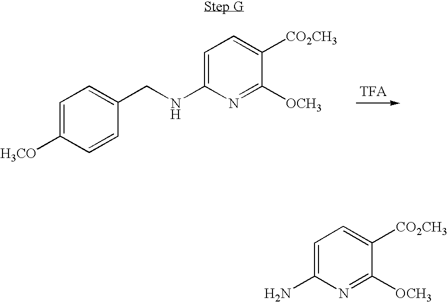

To the 2-Methoxy-6-(4-methoxy-benzylamino)-nicotinic acid methyl ester was added TFA (50 mL) and the mix was warmed to 40° C. for 4 h. Most of the TFA was evaporated off and the residue was suspended in ethyl acetate/20% potassium carbonate and filtered through a Celite® pad. The organic layer was separated, dried with sodium sulfate and evaporated to get 5.4 g (65%) of the product as white solid. Alternatively, upon completion of reaction, the mixture can be added to aqueous NaOAc at 15° C. The solid thus obtained is filtered and dissolved in dichloromethane. Any remaining water is removed and the product is precipitated by adding to heptane.

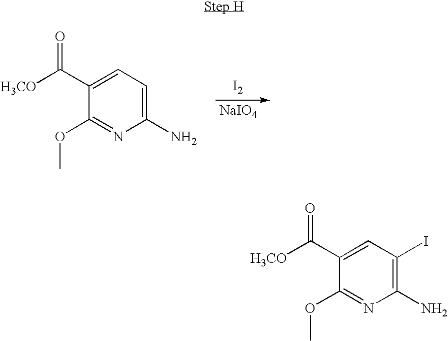

6-Amino-2-methoxy-nicotinic acid methyl ester (20 g, 109.89 mmol) was taken in DMF (100 mL) and iodine (22.4 g, 88 mmol) and NaIO₄ (9.42 g, 44 mmol) were added. The mixture was stirred at 50° C. for 5 h under a nitrogen atmosphere. It was then poured into water and the product was extracted with ethyl acetate. The extract was decolorized using aqueous sodium thiosulphate solution. It was further washed with water, dried over sodium sulfate and concentrated. The crystallized product was collected by filtration. Further concentration of the mother liquor provided another crop to yield 22.25 g of the desired product. LC-MS: 309.

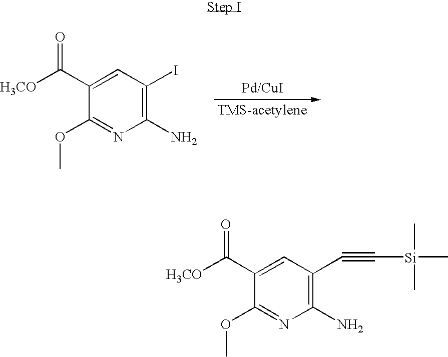

6-Amino-5-iodo-2-methoxy-nicotinic acid methyl ester (19.04 g, 61.82 mmol) was taken in dichloromethane (190 mL) and triethylamine (13 mL, 93 mmol) and Pd(PPh)₂Cl₂ (220 mg, 0.31 mmol) and CuI (411 mg, 2.16 mmol) were added. The mixture was cooled in an ice-bath and trimethylsilylacetylene (9.61 mL, 68 mmol) was added dropwise. The mixture was stirred over ice for another 30 min after which the ice-bath was removed and stirring continued for another 5 h. It was filtered to remove the solids and evaporated to dryness. The product was purified on a column of silica eluting it with ethyl acetate-hexane (0 to 20% ethyl acetate, gradient) to yield 15.69 g of the desired product. LC-MS: 279.

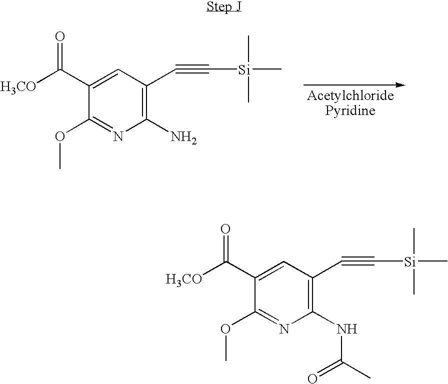

6-Amino-2-methoxy-5-trimethylsilanylethynylnicotinic acid methyl ester (24.63 g, 88.6 mmol) was taken in dichloromethane (250 mL) and pyridine (14.3 mL, 177.2 mmol) was added. The mixture was cooled in an ice-bath and acetyl chloride (7.56 mL, 106.32 mmol) was added dropwise. After 1 h the ice-bath was removed and stirring continued under nitrogen for 20 h. The reaction mixture was washed with water, dried and evaporated. The residue was purified in a column of silica gel eluting with ethyl acetate-hexane (0 to 30% ethyl acetate, gradient) to yield 23.42 g of the desired product. LC-MS: 321.

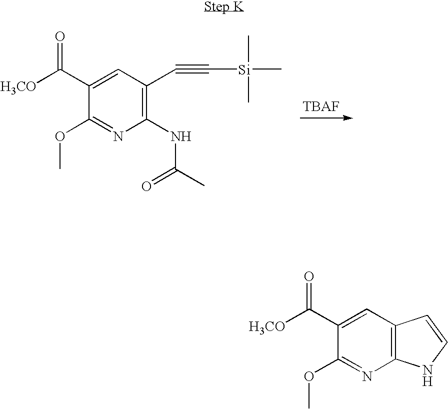

6-Acetylamino-2-methoxy-5-trimethylsilanylethynyl-nicotinic acid methyl ester (18 g, 56.25 mmol) of was dissolved in dry THF (50 mL) and TBAF (1M solution in THF, 225 mL, 225 mmol) was added and the mixture refluxed for 20 h. The volatiles were removed and the residue was extracted with dichloromethane from water. The extract was dried over sodium sulfate, concentrated and purified on a column of silica gel, eluting it with ethyl acetate-hexane (0 to 25% ethyl acetate, gradient) to yield 10.0 g of the desired product. LC-MS: 207.

Alternatively, upon completion of the reaction, the mixture can be cooled and approximately half the solvent removed before purifying on a silica gel column, eluting with THF. The fractions containing the product are concentrated and then purified once again on a silica gel column, eluting with ethyl acetate/heptane (17:8).

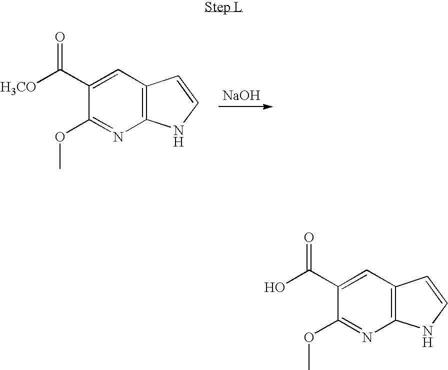

6-Methoxy-1H-pyrrolo[2,3-b]pyridine-5-carboxylic acid methyl ester (2.06 g, 10 mmol) was taken in methanol (50 mL) and 10% aqueous NaOH (16 mL), and water (10 mL) were added. The mixture was then refluxed for 2 h. It was evaporated to remove the methanol, diluted with water and acidified with 10% HCl. The precipitated product was extracted with ethyl acetate, dried over sodium sulfate and evaporated to yield 1.93 g of the desired product. LC-MS: 193.

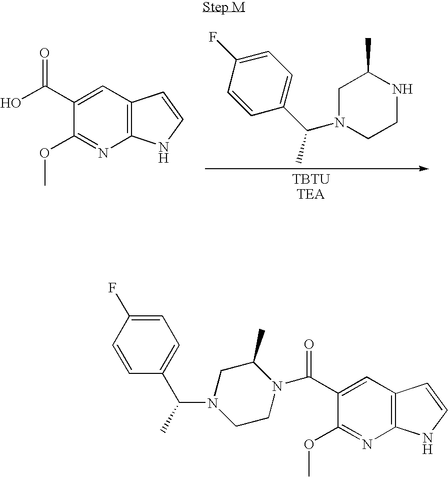

6-Methoxy-1H-pyrrolo[2,3-b]pyridine-5-carboxylic acid (395 mg, 2 mmol) and 1R-[1-(4-fluoro-phenyl)-ethyl]-3R-methyl-piperazine (888 mg, 4 mmol) were dissolved in dry DMF (15 mL) and TBTU (1.28 g, 4 mmol) was added followed by TEA (600 mg, 6 mmol). The mixture was stirred for 20 h. It was poured into water and the product was extracted out with ethyl acetate. The extract was dried, evaporated and purified on a column of silica gel eluting it with ethyl acetate-hexane (20-40% ethyl acetate, Gradient) to yield 510 mg of the desired product. LC-MS: 397.

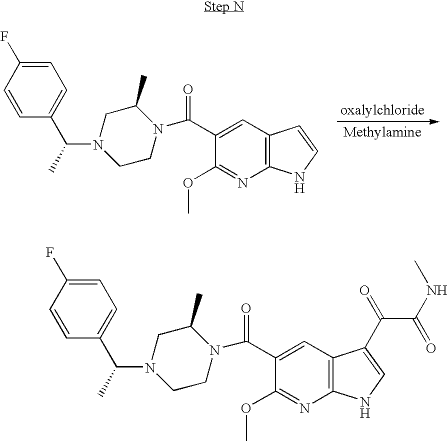

{4-[1R-(4-Fluoro-phenyl)-ethyl]-2R-methyl-piperazin-1-yl}-(6-methoxy-1H-pyrrolo[2,3-b]pyridin-5-yl)-methanone (510 mg, 1.28 mmol) was taken in dry dichloromethane (10 mL) and was cooled in an ice-bath. Oxalylchloride (2 M solution in dichloromethane, 5 mL) was added and the mixture stirred under nitrogen for 1 h. The ice-bath was removed and stirring continued for another 6 h at RT. It was evaporated to dryness and resuspended in dry dichloromethane (15 mL) and was cooled in an ice bath. Methylamine (2 M solution in THF, 5 mL) was added via a syringe and stirring was continued for 30 min. This was poured into water and the product was extracted with dichloromethane. The extract was dried, evaporated and the residue was purified by radial chromatography using CHCl₃-methanol (0 to 3% methanol) as eluant to yield 310 mg of the desired product. M+H⁺483, R_{f}: 1.887 min, Condition B.

Using the same methods described above, the following compound was prepared:

### 2-{5-[4-(4-Fluoro-benzyl)-2R,5S-dimethyl-piperazine-1-carbonyl]-6-methoxy-1H-pyrrolo[2,3-b]pyridin-3-yl}-N-methyl-2-oxo-acetamide

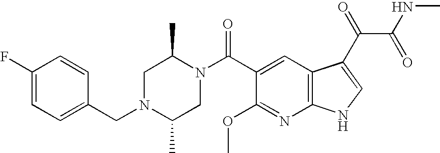

This material was purified by chromatography, eluting with dichloromethane-methanol. It can also be purified by recrystallization from methanol or ethanol. M+H⁺482, R_{f}: 1.847 min, Condition A.

### Example 7

### Preparation of 2-[5-(4-Benzhydryl-2R,5S-dimethyl-piperazine-1-carbonyl)-6-methoxy-1H-pyrrolo[2,3-b]pyridin-3-yl]-N,N-dimethyl-2-oxo-acetamide

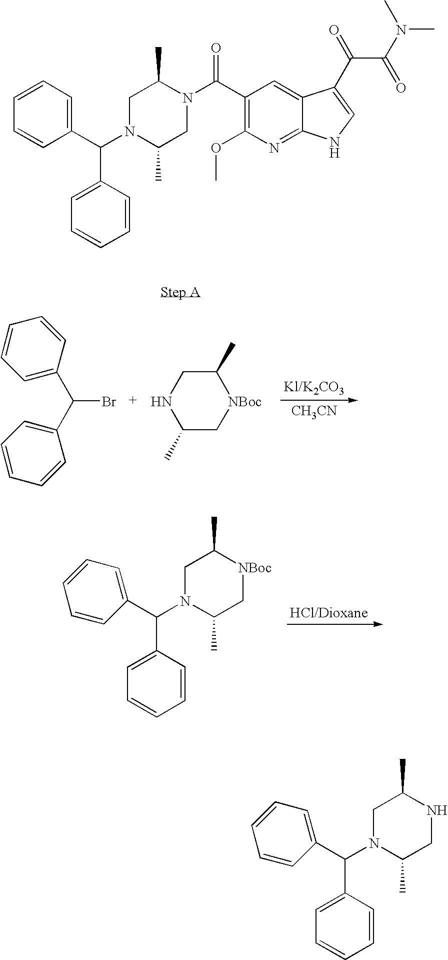

The crude 1-tert-butoxycarbonyl-2S,5R-dimethyl-piperazine was dissolved in acetonitrile (600 mL) and potassium iodide (45.2 g, 272 mmol), potassium carbonate (37.7 g, 272 mmol) and α-bromodiphenylmethane (73.9 g, 299 mmol) were added. The mixture was stirred at room temperature overnight and the solvent was removed. The residue was taken up in EtOAc, washed with 5% potassium carbonate then with brine, dried over sodium sulfate and concentrated. This material was dissolved in 4 M HCl in dioxane and stirred for 1 h. After removal of the solvent, the residue was dissolved in EtOAc, washed with 10% NaOH then with brine, dried over sodium sulfate and concentrated to give crude 1-benzhydryl-2S,5R-dimethyl-piperazine which was purified using flash chromatography (EtOAc/hexanes) to give 3 7 g pure 1-benzhydryl-2S,5R-dimethyl-piperazine. M+H⁺(281).

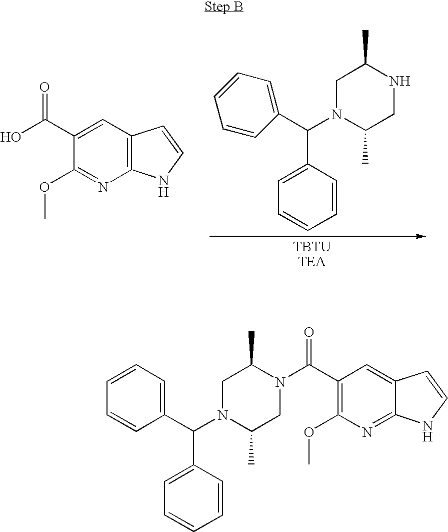

Prepared from 6-Methoxy-1H-pyrrolo[2,3-b]pyridine-5-carboxylic acid and 1-Benzhydryl-2S,5R-dimethyl-piperazine according to the procedure described in Example 6, Step M. M+H⁺(455).

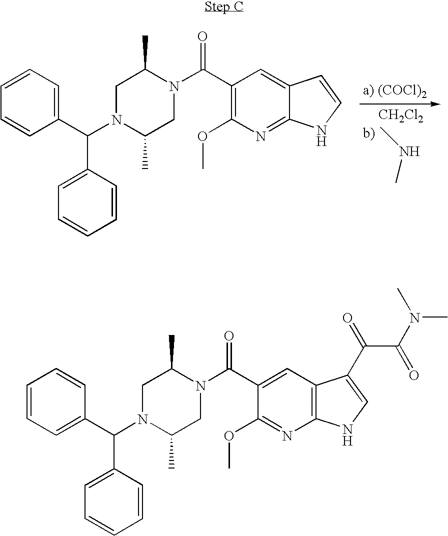

Prepared according to the procedure in Example 1, Step I using dimethylamine in place of pyrrolidine. M+H⁺553, R_{f}: 2.467 min, Condition A.

The following compounds were prepared by the methods illustrated in the preceding examples:

### 2-[5-(4-Benzhydryl-2R,5S-dimethyl-piperazine-1-carbonyl)-6-methoxy-1H-pyrrolo[2,3-b]pyridin-3-yl]-N-methyl-2-oxo-acetamide

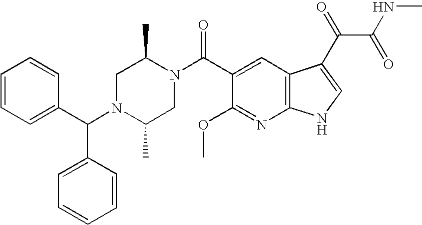

M+H⁺539, R_{f}: 2.487 min, Condition A.

### 1-[5-(4-Benzhydryl-2R,5S-dimethyl-piperazine-1-carbonyl)-6-methoxy-1H-pyrrolo[2,3-b]pyridin-3-yl]-2-pyrrolidin-1-yl-ethane-1,2-dione

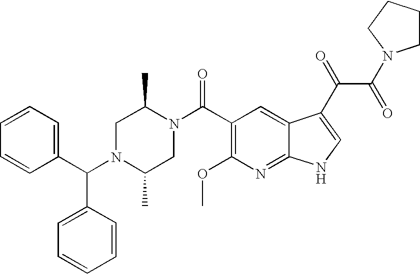

M+H⁺579, R_{f}: 2.607 min, Condition A.

### 2-[5-(4-Benzhydryl-2R,5S-dimethyl-piperazine-1-carbonyl)-6-methoxy-1H-pyrrolo[2,3-b]pyridin-3-yl]-N-cyclopentyl-2-oxo-acetamide

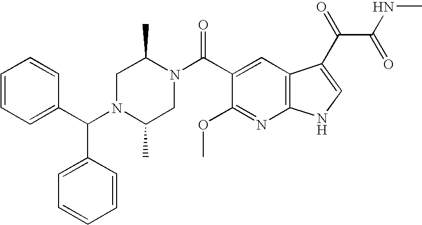

M+H⁺593, R_{f}: 3.067 min, Condition A.

### 2-{5-[4-(4-Fluoro-benzyl)-2R-methyl-piperazine-1-carbonyl]-6-methoxy-1H-pyrrolo[2,3-b]pyridin-3-yl}-N-methyl-2-oxo-acetamide

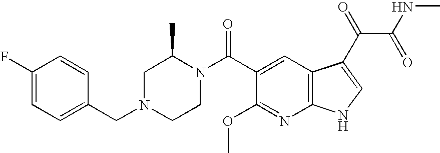

M+H⁺468, R_{f}: 1.747 min, Condition A.

### 2-{5-[4-(4-Fluoro-benzyl)-2R-methyl-piperazine-1-carbonyl]-6-methoxy-1H-pyrrolo[2,3-b]pyridin-3-yl}-2-oxo-acetamide

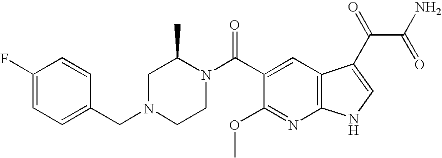

M+H⁺454, R_{f}: 1.707 min, Condition A.

### Example 8

### Preparation of 2-{5-[4-(4-Fluoro-benzyl)-2R,5S-dimethyl-piperazine-1-carbonyl]-6-ethoxy-1H-pyrrolo[2,3-b]pyridin-3-yl}-N-ethyl-2-oxo-acetamide

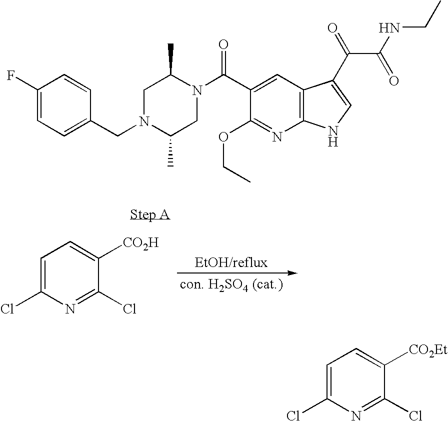

To a solution of 2,6-dichloronicotinic acid (37 g) in 200 mL ethanol was added 4 mL of con. H₂SO₄ and the mixture was refluxed overnight. The ethanol was evaporated off and the residue was dissolved in ethyl acetate, washed with water, 10% sodium carbonate solution, brine, dried with sodium sulfate, filtered and evaporated to yield the desired product (37.29 g) as white solid (R_{f}: 1.553 min., Condition B, M+H⁺: 220).

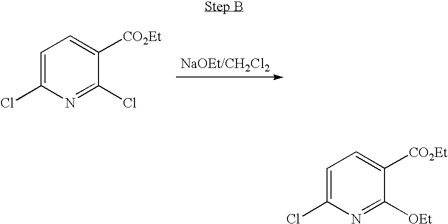

To a solution of 2,6-dichloro-nicotinic acid ethyl ester (37.29 g) in dichloromethane (200 mL) at −2° C. was added NaOEt (17.36 g) slowly and stirred at −2° C. for 1 h and then at 3° C. for 3 h. The reaction mixture was diluted with water, the organic layer was dried with sodium sulfate, filtered and evaporated to give white solid (31.6 g) (R_{f}: 1.78 min., Condition B, M+H⁺: 230).

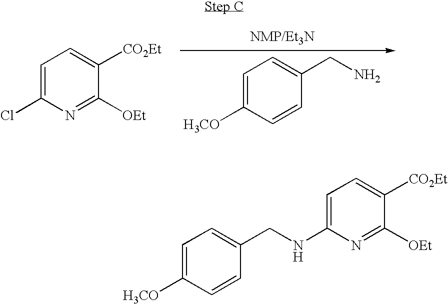

To a solution of 6-chloro-2-ethoxy-nicotinic acid ethyl ester (31.6 g) in 55 mL of NMP was added p-methoxybenzylamine (27 mL) and triethylamine (20.7 mL). The mixture was heated to 70° C. overnight. It was cooled and diluted with water and extracted with ethyl acetate, washed with water, brine, dried with sodium sulfate, filtered and evaporated. The residue obtained was triturated with ethylacetate/hexane. This was then filtered and dried to yield 32.3 g of the target compound (R_{f}: 1.94 min., Condition B, M+H⁺: 331).

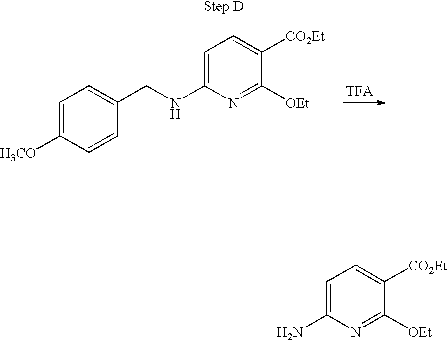

To the 2-ethoxy-6-(4-methoxy-benzylamino)-nicotinic acid ethyl ester (32.3 g) was added TFA (110 mL) and the mixture was warmed to 40° C. for 5 h. Most of the TFA was evaporated off and the residue was suspended in ethyl acetate/20% potassium carbonate. The organic layer was separated, dried with sodium sulfate, filtered and evaporated. The residue obtained was triturated with ethyl acetate. The solid thus separated was then filtered and dried to yield 13.2 g of the target compound (R_{f}: 1.067 min., Condition B, M+H⁺: 211).

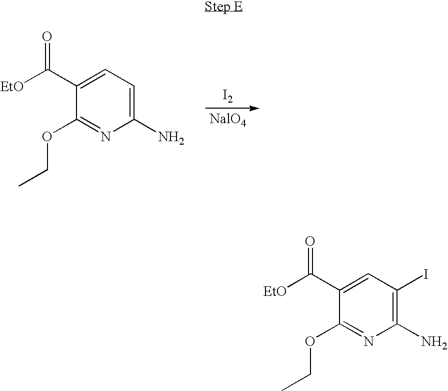

6-Amino-2-ethoxy-nicotinic acid ethyl ester (13.2 g) was taken in DMF (100 mL) and iodine (12.76 g) and NaIO₄ (5.37 g) were added. The mixture was stirred at 50° C. for 8 h under a nitrogen atmosphere. The mixture was then poured into aqueous sodium metabisulfite solution. The solid thus separated was filtered, washed with water and dried under high vacuum overnight to give 18.2 g of the desired product (R_{f}: 1.54 min., Condition B, M+H⁺: 337).

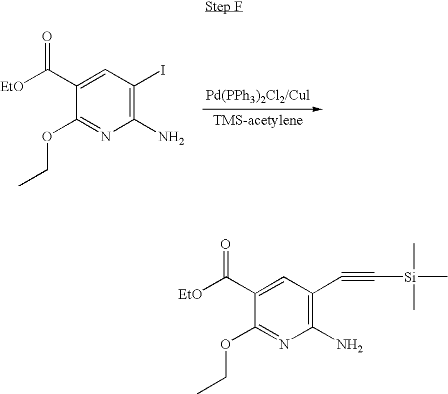

6-Amino-5-iodo-2-ethoxy-nicotinic acid ethyl ester (18.2 g) was taken in dichloromethane (150 mL) and triethylamine (11.28 mL) and Pd(PPh₃)₂Cl₂ (378.56 mg) and CuI (721.8 mg) were added. The mixture was cooled in an ice-bath and trimethylsilylacetylene (8.37 mL) was added dropwise. The mixture was stirred at room temperature for 5 h. The reaction mixture was concentrated and the residue obtained was diluted with water and ethyl acetate. The layers were filtered through a Celite® pad and the organic layer was separated, dried over sodium sulfate, filtered and concentrated. The compound was purified by flash chromatography using 20% ethyl acetate/hexane to 30% ethyl acetate/hexane as a solvent (Yield: 14.7 g, R_{f}: 2.00 min., Condition B, M+H⁺: 307).

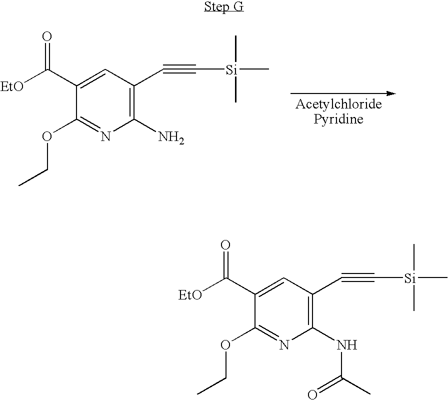

6-Amino-2-ethoxy-5-trimethylsilanylethynylnicotinic acid ethyl ester (14.7 g) was taken in dichloromethane (175 mL) and pyridine (7.8 mL) was added. The mixture was cooled in an ice-bath and acetyl chloride (4 mL) was added dropwise. The reaction was stirred at room temperature overnight. The reaction mixture was diluted with water and ethyl acetate. The organic layer was separated, dried over sodium sulfate, filtered and evaporated. The residue obtained was triturated with hexane. The solid thus separated was filtered and dried under high vacuum overnight (Yield: 13 g, R_{f}: 2.007 min., Condition B, M+H⁺: 349).

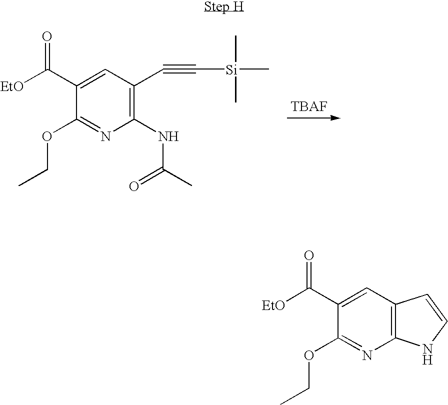

6-Acetylamino-2-ethoxy-5-trimethylsilanylethynyl-nicotinic acid ethyl ester (13 g) was dissolved in dry THF (20 mL) and TBAF (1M solution in THF, 150 mL) was added and the mixture refluxed for 4 h. The volatiles were removed and the residue was diluted with water. The solid thus separated was filtered, washed with water and dried under high vacuum overnight (Yield: 8.3 g, R_{f}: 1.36 min., Condition B, M+H⁺: 235).

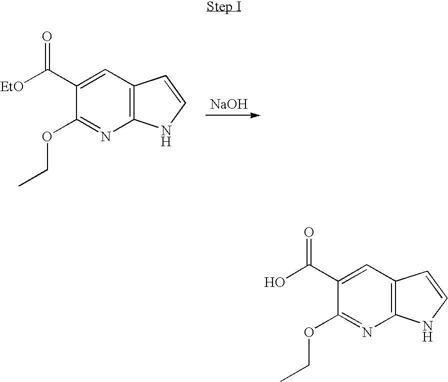

6-Ethoxy-1H-pyrrolo[2,3-b]pyridine-5-carboxylic acid ethyl ester (8.3 g) was taken in ethanol (90 mL), NaOH (4.2 g), and water (90 mL) were added. The mixture was then stirred at 70° C. for 2 h. It was evaporated to remove the ethanol, diluted with water and acidified with concentrated HCl to pH 2. The solid thus separated was filtered, washed with water and dried under high vacuum overnight (Yield: 7 g, R_{f}: 0.94 min., Condition B, M+H⁺: 207).

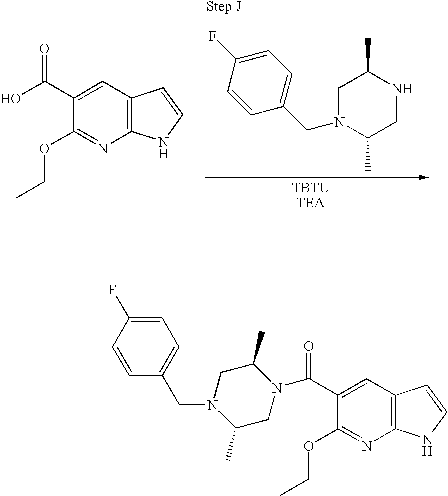

6-Ethoxy-1H-pyrrolo[2,3-b]pyridine-5-carboxylic acid (3.0 g) and 1-[1-(4-fluoro-phenyl)-ethyl]-2S,5R-dimethyl-piperazine (3.23 g) were dissolved in dry DMF (20 mL) and TBTU (4.67 g) was added followed by TEA (6.1 mL). The mixture was stirred for 5 h. It was poured into water and the product thus separated was filtered, washed with water and dried under high vacuum overnight (Yield: 6 g, R_{f}: 1.04 min, Condition B, M+H⁺: 411).

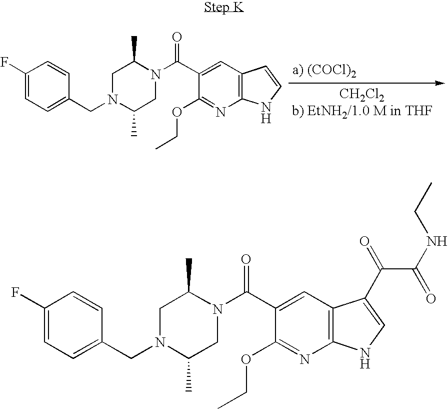

4-(4-Fluoro-benzyl)-2R,5S-dimethyl-piperazin-1-yl-(6-ethoxy-1H-pyrrolo[2,3-b]pyridin-5-yl)-methanone (170 mg) in anhydrous CH₂Cl₂ (5 mL) was added oxalyl chloride (0.82 mL, 2 M in CH₂Cl₂) at RT. The reaction mixture was stirred at room temperature for 5 h. The reaction mixture was concentrated under reduced pressure. The residue was dried under vacuum for 1 h and dissolved in CH₂Cl₂. An excess amount of ethyl amine (1 mL, 1.0 M in THF) was added to the reaction mixture. Stirred for 1 h, the reaction mixture was treated with water. The organic layer was separated and washed with brine, dried and concentrated. The residue was purified by preparative chromatography (R_{f}: 1.07 min, Condition B, M+H⁺: 510).

The following compounds were prepared by the same general method:

### 2-{5-[4-(4-Fluoro-benzyl)-2R,5S-dimethyl-piperazine-1-carbonyl]-6-ethoxy-1H-pyrrolo[2,3-b]pyridin-3-yl}-N-methyl-2-oxo-acetamide

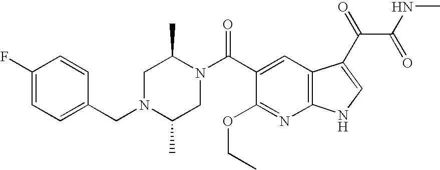

R_{f}: 1.953 min, Condition A, M+H⁺: 496.

### 2-{6-Ethoxy-5-[4-(4-fluoro-benzyl)-2R,5S-dimethyl-piperazine-1-carbonyl]-1H-pyrrolo[2,3-b]pyridin-3-yl}-2-oxo-acetamide

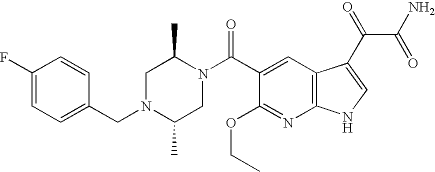

R_{f}: 1.927 min, Condition A, M+H⁺: 483.

### 2-{6-Ethoxy-5-[4-(4-fluoro-benzyl)-piperidine-1-carbonyl]-1H-pyrrolo[2,3-b]pyridin-3-yl}-N-methyl-2-oxo-acetamide

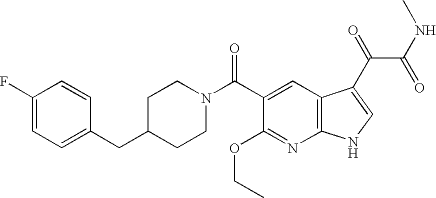

R_{f}: 3.340 min, Condition A, M+H⁺: 467.

### 2-{6-Ethoxy-5-[4-(4-fluoro-benzyl)-piperidine-1-carbonyl]-1H-pyrrolo[2,3-b]pyridin-3-yl}-2-oxo-acetamide

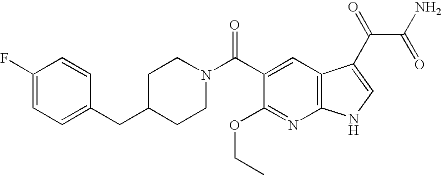

R_{f}: 3.600 min, Condition A, M+H⁺: 454.

### 2-{5-[4-(4-fluoro-benzyl)-2R,5S-dimethyl-piperazine-1-carbonyl]-6-ethoxy-1H-pyrrolo[2,3-b]pyridin-3-yl}-N-isopropyl-2-oxo-acetamide

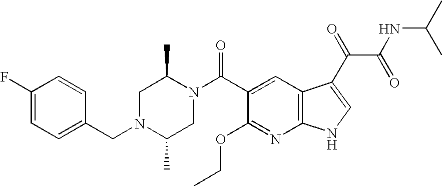

R_{f}: 1.127 min, Condition B, M+H⁺: 524.

### 1-{6-Ethoxy-5-[4-(4-fluoro-benzyl)-2,5-dimethyl-piperazine-1-carbonyl]-1H-pyrrolo[2,3-b]pyridin-3-yl}-2-pyrrolidin-1-yl-ethane-1,2-dione

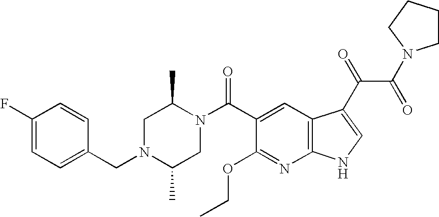

R_{f}: 1.067 min, Condition B, M+H⁺: 536.

### Example 9

### Preparation of 2-{5-[4-(4-Fluoro-benzyl)-2R,5S-dimethyl-piperazine-1-carbonyl]-6-methyl-1H-pyrrolo[2,3-b]pyridin-3-yl}-N-methyl-2-oxo-acetamide

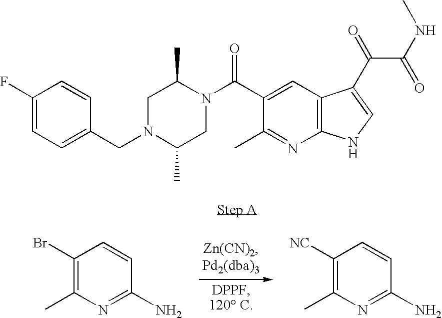

To the degassed N,N-dimethylformamide (150 mL) solution of 5-bromo-6-methyl-2-aminopyridine (25 g), Zn(CN)₂ (9.425 g, 0.6 equiv.) and 1,1-bis(diphenylphosphino)ferrocene (DPPF) (7.425 g, 0.1 equiv.) was added Pd₂(dba)₃ (3.075 g, 0.025 equiv.). The reaction was heated to 130° C. in a sealed tube for 4 days. The reaction was then cooled to 85° C. and diluted with 90 mL NH₄Cl (saturated), 90 mL H₂O and 22.5 mL NH₄OH (4:4:1). The mixture was then stirred overnight at 85° C. After cooling to room temperature, the reaction mixture was extracted with ethyl acetate. The two layers were then filtered through Celite®. The organic layer was separated, washed with water, brine, and then dried over Na₂SO₄. After removing the volatiles, the residue was triturated with ethyl acetate. The solid thus separated was filtered and dried under high vacuum overnight. The filtrate was purified by silica gel chromatography eluting with 30 to 100% ethyl acetate in hexane (Yield: 13 g, R_{f}: 0.273 min, Condition B, M+H⁺: 134).

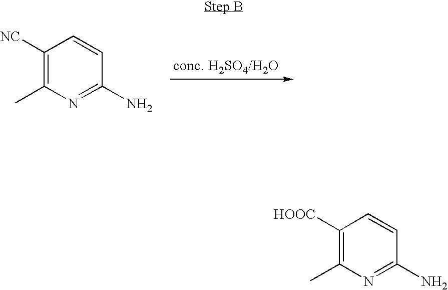

The 6-amino-2-methyl-3-cyanopyridine (4.1 g) was added to the sulfuric acid (10 mL) slowly with stirring. The reaction mixture was stirred at 100° C. for 1 h in a sealed tube. The reaction mixture was diluted with 10 mL of water and reaction was continued for 5 h. The reaction mixture was cooled and maintained at room temperature overnight. The solid thus separated was filtered, washed with cold water and was dried under high vacuum pump overnight (Yield: 4.5 g, R_{f}: 0.227 min, Condition B, M+H⁺: 153).

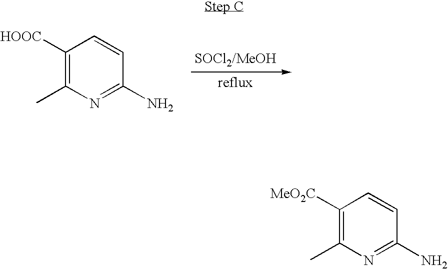

The 6-Amino-2-methyl-nicotinic acid (5.8 g) was dissolved in methyl alcohol (100 mL) and then cooled to 0° C. in an ice-water bath. Thionyl chloride (9 mL) was then added to the solution. The reaction mixture was refluxed for 8 h. After evaporating the methanol, the residue was neutralized with saturated solution of sodium bicarbonate and was extracted with ethyl acetate. The organic layer was washed with water, brine, dried over sodium sulfate and filtered. The filtrate was concentrated to give 4.46 g of the ester (R_{f}: 0.473 min, Condition B, M+H⁺: 167).

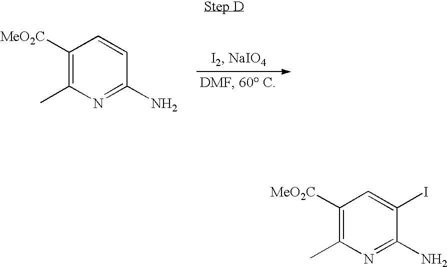

To a N,N-dimethylformamide (20 mL) solution of 6-Amino-2-methyl-nicotinic acid methyl ester (1.65 g) was added iodine (2.02 g) and sodium metaperiodate (0.85 g). The reaction was then heated at 60° C. for 48 h. After cooling to room temperature, the reaction was poured into the solution of sodium metabisulfite. The solid thus separated was filtered, washed with water and dried under high vacuum overnight (Yield: 1.8 g, R_{f}: 0.74 min, Condition B, M+H⁺: 293).

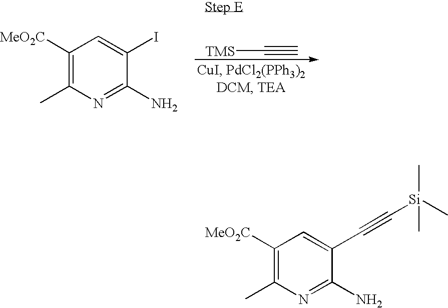

To the dichloromethane solution of 6-amino-5-iodo-2-methyl-nicotinic acid methyl ester (1.8 g) was added copper iodide (82 mg), PdCl₂(PPh₃)₂ (43.2 mg), triethylamine (1.3 mL) and trimethylsilyl acetylene (0.96 mL) sequentially. The reaction was stirred at room temperature for 5 h. The reaction mixture was concentrated and the residue was dissolved in ethyl acetate and water. The two layers were filtered through a Celite® pad. The ethyl acetate layer was separated, washed with water and brine then dried over Na₂SO₄. The solvent was evaporated and the residue was purified by silica gel chromatography using 20-100% ethyl acetate/hexane as a solvent (Yield: 1.3 g, R_{f}: 1.38 min, Condition B, M+H⁺: 263).

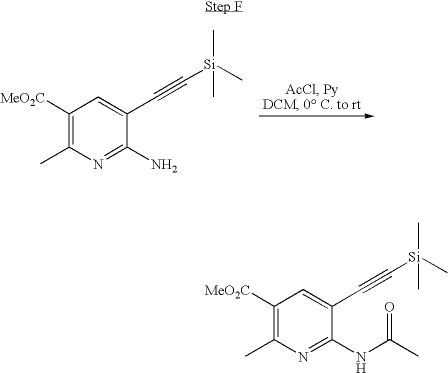

At 0° C., the acetyl chloride (0.41 mL) was added to the dichloromethane (15 mL) solution of 6-amino-5-trimethylsilylacetylenyl-2-methyl-nicotinic acid methyl ester (1.3 g) and pyridine (0.8 mL). The reaction was stirred at room temperature for 7 h, whereupon it was quenched with water. The dichloromethane layer was separated, dried over sodium sulfate and filtered. The filtrate was concentrated under vacuum and the residue obtained was dried under high vacuum and was used as such for the next step without any purification (Yield: 1.5 g, R_{f}: 1.66 min, Condition B, M+H⁺: 305).

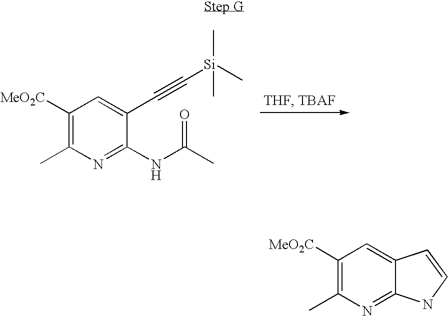

Tetrabutylammonium fluoride (TBAF, 8 mL, 1.0 M/THF) was added to the tetrahydrofuran (5 mL) solution of 6-acetylamino-2-methyl-5-trimethylsilanylethynyl-nicotinic acid methyl ester (1.5 g). The reaction was stirred at 70° C. for 4 h. The reaction mixture was cooled to room temperature and was concentrated. The residue obtained was diluted with water. The solid thus separated was filtered and was washed with water. The solid was dried under high vacuum and was used as such for the next step without any purification (Yield: 0.85 g, R_{f}: 0.8 min, Condition B, M+H⁺: 191).

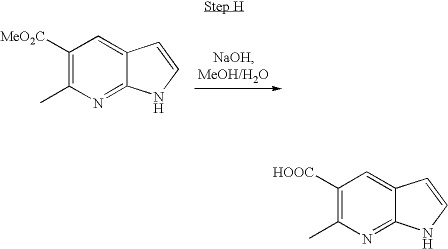

To the methanol (10 mL) and water (10 mL) suspension of6-methyl-1H-pyrrolo[2,3-b]pyridine-5-carboxylic acid methyl ester (0.85 g) was added sodium hydroxide (0.53 g). The reaction was heated at 70° C. for 2 h. The reaction mixture was concentrated and the residue obtained was redissolved in water. The aqueous solution was acidified with conc. hydrochloric acid to pH 2. The solid thus separated was filtered, washed with water and dried under high vacuum. It was used as such for the next step without any purification (Yield: 0.65 g, R_{f}: 0.48 min, Condition B, M+H⁺: 177).

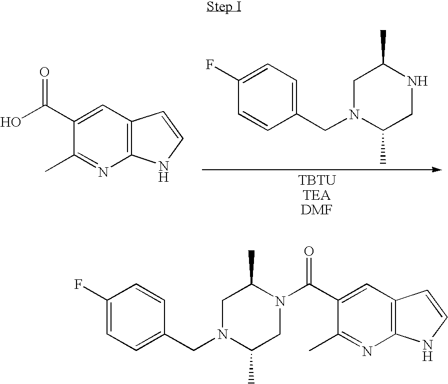

6-Methyl-1H-pyrrolo[2,3-b]pyridine-5-carboxylic acid (0.65 g) and 4-fluorobenzyl-2S,5R-dimethyl piperazine (0.82 g) were dissolved in dry DMF (10 mL) and TBTU (1.18 g) was added followed by triethylamine(1.54 mL). The mixture was stirred for 5 h, whereupon it was poured into water and the solid thus separated was filtered and dried. The crude was purified by silica gel chromatography using ethyl acetate as a solvent (Yield: 0.87 g, R_{f}: 0.767 min, Condition B, M+H⁺: 380).

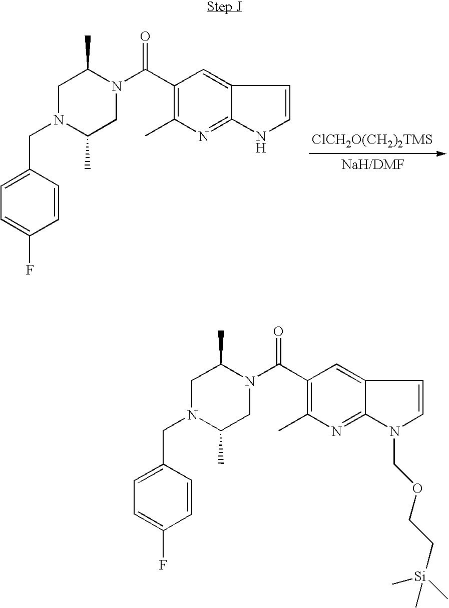

To the solution of N,N-dimethylformamide (5 mL) and sodium hydride (39 mg, 60% in oil) at 0° C. was added a solution of [4-(4-fluoro-benzyl)-2R,5S-dimethyl-piperazin-1-yl]-(6-methyl-1H-pyrrolo[2,3-b]pyridin-5-yl)-methanone (0.336 g) in N,N-dimethylformamide (1 mL). The reaction was stirred at room temperature for 1 h. The reaction was cooled to 0° C. and to this was added 2-(trimethylsilyl)ethoxymethyl chloride (0.172 mL). The reaction was continued at room temperature for 4 h and was quenched with water followed by extraction with ethyl acetate. The organic layer was separated, dried over sodium sulfate, filtered and concentrated. The crude material was purified by silica gel chromatography using 40% ethyl acetate/hexane as a solvent (Yield: 0.394 g, R_{f}: 1.513 min, Condition B, M+H⁺: 511).

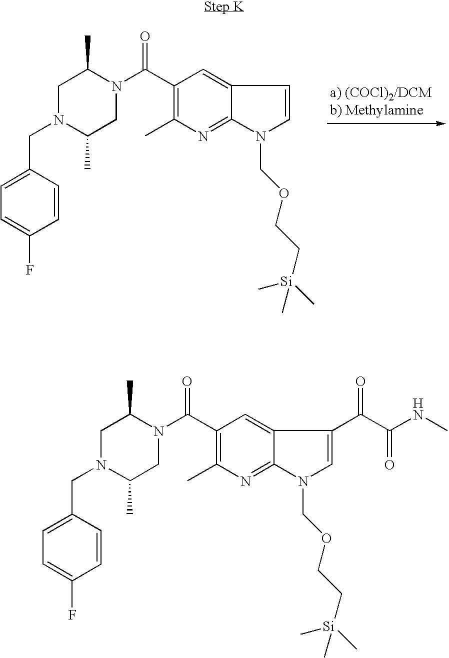

[4-(4-Fluoro-benzyl)-2R,5S-dimethyl-piperazin-1-yl]-[6-methyl-1-(2-trimethylsilanyl-ethoxymethyl)-1H-pyrrolo[2,3-b]pyridin-5-yl]-methanone (0.394 g) was taken in dry dichloromethane (5 mL) and oxalyl chloride (2 M solution in dichloromethane, 1.6 mL) was added and the mixture was stirred under nitrogen overnight. It was evaporated to dryness and dried under high vacuum for 30 min. The residue was re-suspended in dry dichloromethane (5 mL) and methylamine (2 M solution in THF, 2.3 mL) was added via syringe and stirring was continued for 30 min. This was poured into water and the product was extracted with dichloromethane. The extract was dried, evaporated and the residue was purified by radial chromatography using ethyl acetate as eluant to yield 0.25 g of the desired product (R_{f}: 1.48 min, Condition B, M+H⁺: 596).

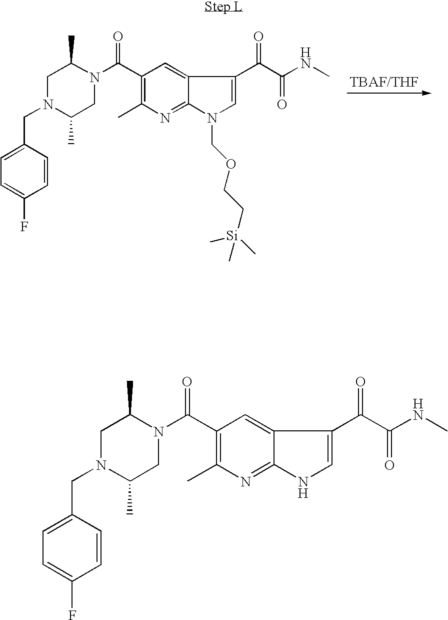

Tetrabutylammonium fluoride (5 mL, 1.0M/THF) was added to the 2-[5-[4-(4-Fluoro-benzyl)-2R,5S-dimethyl-piperazine-1-carbonyl]-6-methyl-1-(2-trimethylsilanyl-ethoxymethyl)-1H-pyrrolo[2,3-b]pyridin-3-yl]-N-methyl-2-oxo-acetamide (0.1 g). Reaction was stirred at 60° C. for 3 h. The reaction mixture was cooled to room temperature and was concentrated. The residue obtained was diluted with water. The solid thus separated was filtered and washed with water. The solid was purified by preparative HPLC (Yield: 0.045 g, R_{f}: 0.887 min, Condition B, M+H⁺: 466).

The following compounds were prepared by the same general method:

### 2-{5-[4-(4-Fluoro-benzyl)-2R,5S-dimethyl-piperazine-1-carbonyl]-6-methyl-1H-pyrrolo[2,3-b]pyridin-3-yl}-N,N-dimethyl-2-oxo-acetamide

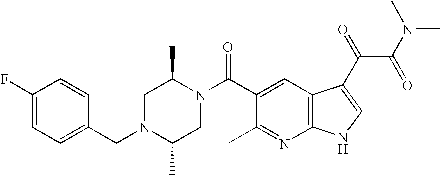

R_{f}: 0.93 min, Condition B, M+H⁺: 481.

### N-Ethyl-2-{5-[4-(4-fluoro-benzyl)-2R,5S-dimethyl-piperazine-1-carbonyl]-6-methyl-1H-pyrrolo[2,3-b]pyridin-3-yl}-2-oxo-acetamide

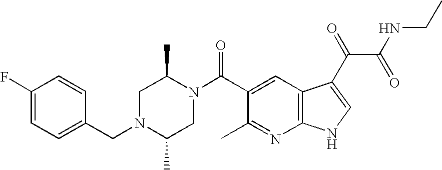

R_{f}: 0.96 min, Condition B, M+H⁺: 481.

### 1-{5-[4-(4-Fluoro-benzyl)-2R,5S-dimethyl-piperazine-1-carbonyl]-6-methyl-1H-pyrrolo[2,3-b]pyridin-3-yl}-2-pyrrolidin-1-yl-ethane-1,2-dione

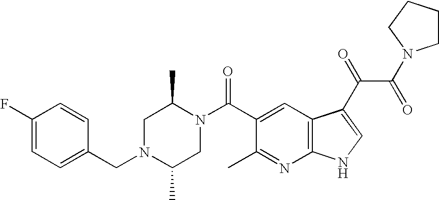

R_{f}: 1.00 min, Condition B, M+H⁺: 507.

### 2-{5-[4-(4-Fluoro-benzyl)-2R,5S-dimethyl-piperazine-1-carbonyl]-6-methyl-1H-pyrrolo[2,3-b]pyridin-3-yl}-2-oxo-acetamide

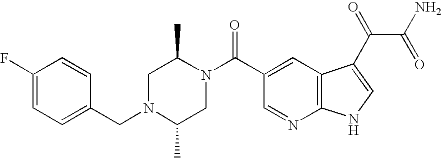

R_{f}: 0.873 min, Condition B, M+H⁺: 452.

### Example 10

### Preparation of 1-{5-[4-(4-Fluoro-benzyl)-piperidine-1-carbonyl]-6-methoxy-2-methyl-1H-pyrrolo[2.3-b]pyridin-3-yl}-2-pyrrolidin-1-yl-ethane-1,2-dione

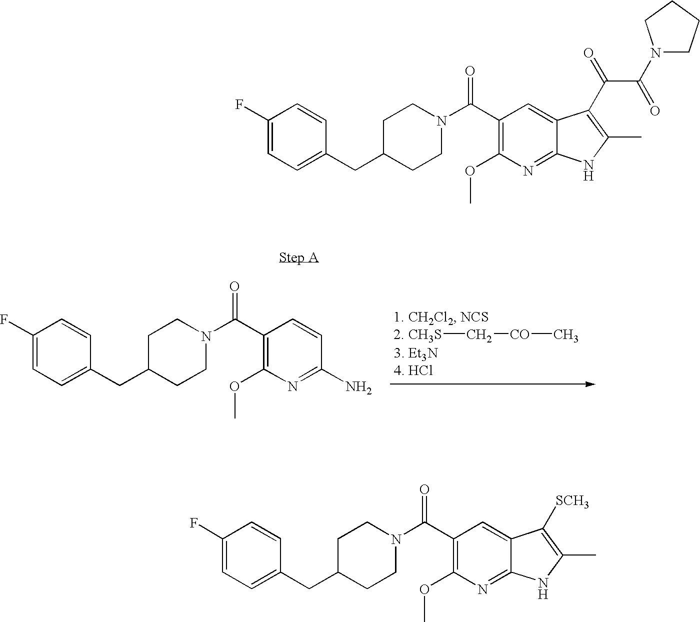

The starting material was prepared as in Step D in Example 1. The azaindole was synthesized using the method of Gassman (Paul G. Gassman et. al. J. Amer. Chem. Soc. (1974), 96, 5495-5507). 6-Methoxy-2-amino pyridine carboxamide (0.9 g, 2.63 mMol) was dissolved in CH₂Cl₂ (10 mL) and was treated with N-chlorosuccinimide (NCS) (0.42 g, 3.16 mMol) at −40° C. for 3 h. Thiomethylacetone was added (0.27 g, 2.63 mMol) followed by Et₃N (0.32 g, 3.1 mmol). The mixture was then evaporated and treated with 10% HCl in methanol. The indole product (70 mg) was obtained after silica gel chromatography using hexane/ethyl acetate (1:1). LC-MS 428, M+1.

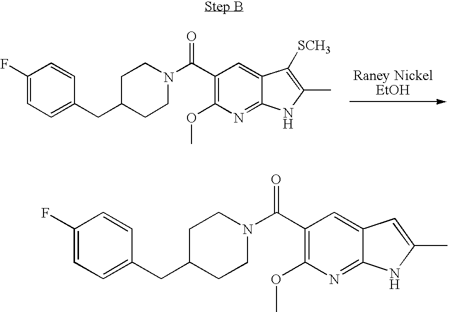

To a solution of thiomethyl azaindole (70 mg) in 10 mL of ethanol was added Raney Nickel in portions over a period of 2 h. The suspension was filtered through a Celite® pad and evaporated to get an oil. The product was obtained (20 mg) after purification by silica gel chromatography using hexane/ethyl acetate. LC-MS 381, M+1.

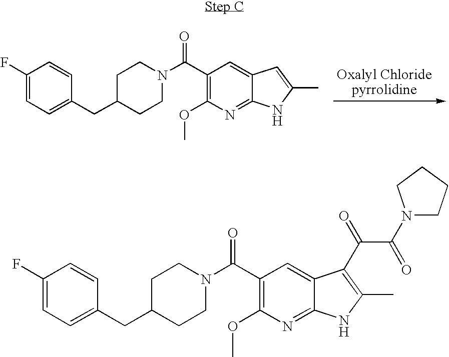

To a solution 2-methylazaindole (20 mg, 0.05 mmol) in 1 mL CH₂Cl₂ was added oxalyl chloride (1.0 mmol) and stirred for 4 h. The reaction mixture was concentrated and the residue was thoroughly dried. This residue was dissolved in CH₂Cl₂ and treated with excess pyrrolidine. After purification using silica gel chromatography using methylene chloride/methanol (1:0.1), the final product (24 mg) was obtained as a white powder (R_{f}: 3.19 min, Condition A, M+H⁺: 507).

### Example 11

### Preparation of 2-{5-[4-(4-Fluoro-benzyl)-2R,5S-dimethyl-piperazine-1-carbonyl]-1H-pyrrolo[3,2-b]pyridin-3-yl}-N-methyl-2-oxo-acetamide

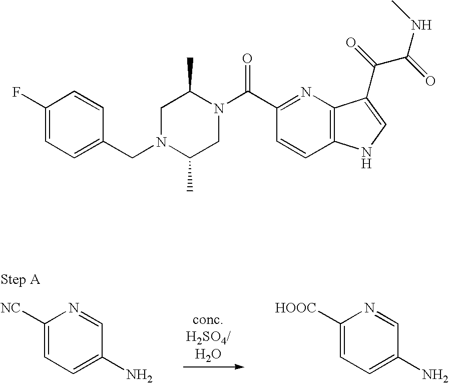

The 5-amino-2-cyanopyridine (4.0 g) was added to sulfuric acid (20 mL) slowly with stirring. The reaction mixture was stirred at 90° C. for 2 h in a sealed tube. The reaction mixture was diluted with water (40 mL) and heating was continued at 100° C. for 2 h. The reaction mixture was cooled to room temperature and was poured into ice. The solid thus separated was filtered, washed with cold water and was dried under vacuum to yield 6.4 g of the desired product (R_{f}: 0.173 min, Condition B, M+H⁺: 139).

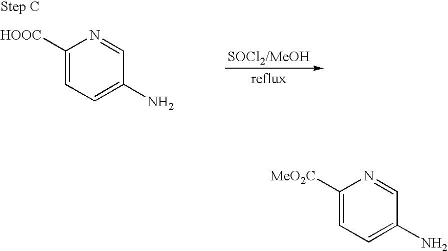

5-Amino-2-nicotinic acid (6.38 g) was dissolved in methyl alcohol (50 mL) and then cooled to 0° C. in an ice-water bath. Thionyl chloride (8.3 mL) was then added to the solution. The reaction mixture was refluxed for 30 h. After evaporating the methanol, the residue was neutralized with a saturated solution of sodium bicarbonate and was extracted with ethyl acetate. The organic layer was washed with water, brine, dried over sodium sulfate and filtered. The filtrate was concentrated to give 2.31 g of the final ester (R_{f}: 0.473 min, Condition B, M+H⁺: 153).

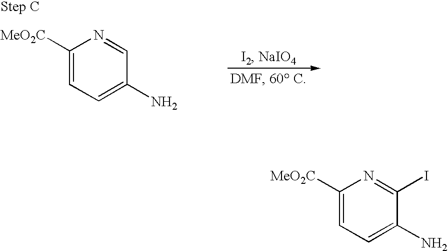

To a N,N-dimethylformamide (20 mL) solution of 5-Amino-2-nicotinic acid methyl ester (2.31 g) was added iodine (3.09 g) and sodium metaperiodate(1.3 g). The reaction was then heated to 60° C. for 24 h. After cooling to room temperature, the reaction was poured into a solution of sodium metabisulfite. The solid thus separated was filtered, washed with water and dried under vacuum to yield 1.6 g of the desired compound (R_{f}: 0.84 min, Condition B, M+H⁺: 279).

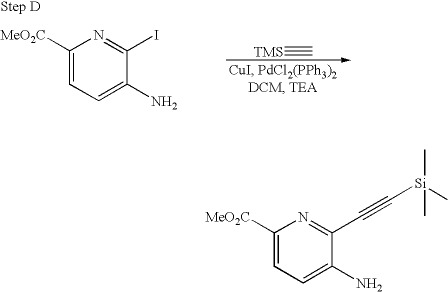

To a dichloromethane solution of 5-amino-6-iodo-2-nicotinic acid methyl ester (1.6 g) was added copper iodide (77 mg), PdCl₂(PPh₃)₂ (40 mg), triethylamine (1.2 mL) and trimethylsilyl acetylene (0.9 mL) sequentially. The reaction was stirred at room temperature for 3 h and then the reaction mixture was concentrated and the residue was dissolved in ethyl acetate and water. The two layers were filtered through a Celite® pad. The ethyl acetate layer was separated, washed with water and brine then dried over Na₂SO₄. The solvent was evaporated and the residue was purified by flash chromatography using 30-100% ethyl acetate/hexane as a solvent, yielding 1.2 g of the product (R_{f}: 1.39 min, Condition B, M+H⁺: 249).

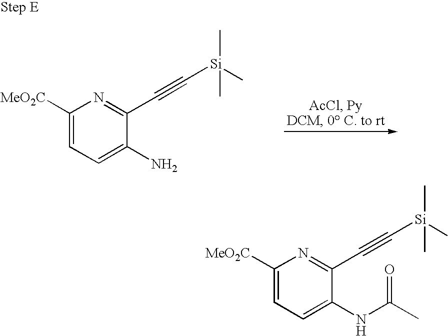

At 0° C., acetyl chloride (0.4 mL) was added to the dichloromethane (20 mL) solution of 5-amino-6-trimethylsilylacetylene-2-nicotinic acid methyl ester (1.2 g) and pyridine (0.78 mL). The reaction was stirred at room temperature for 1 h. The reaction was quenched with water. Dichloromethane layer was separated, dried over sodium sulfate and filtered. The filtrate was concentrated under vacuum. The residue obtained was dried at high vacuum and was used as such for the next step without any purification. (Yield: 1.46 g, R_{f}: 1.577 min, Condition B, M+H⁺: 291).

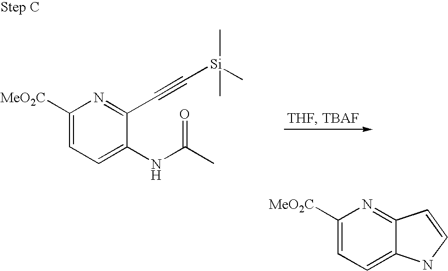

Tetrabutylammonium fluoride (6 mL, 1.0 M in THF) was added to the tetrahydrofuran (7 mL) solution of 5-acetylamino-5-trimethylsilanylethynyl-2-nicotinic acid methyl ester (1.46 g). The reaction was stirred at 70° C. for 4 h then the reaction mixture was cooled to room temperature and was concentrated. The residue obtained was diluted with water. The solid thus separated was filtered and was washed with water. The solid was dried under high vacuum and was used as such for the next step without any purification (Yield: 0.69 g, R_{f}: 0.37 min, Condition B, M+H⁺: 177).

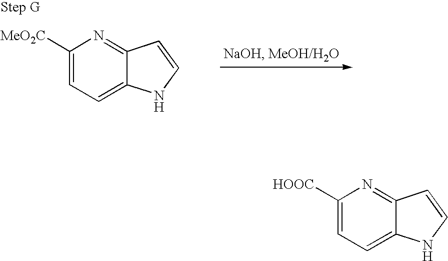

To the methanol (10 mL) and water (10 mL) suspension of 1H-pyrrolo[3,2-b]pyridine-5-carboxylic acid methyl ester (0.54 g) was added sodium hydroxide (0.37 g). The reaction was heated at 70° C. for 2 h. The reaction mixture was concentrated and the residue obtained was redissolved in water. The aqueous solution was acidified with conc. hydrochloric acid to pH 2. The solid thus separated was filtered, washed with water and dried under high vacuum. It was used as such for the next step without any purification. (Yield: 0.49 g, R_{f}: 0.213 min, Condition B, M+H⁺: 163).

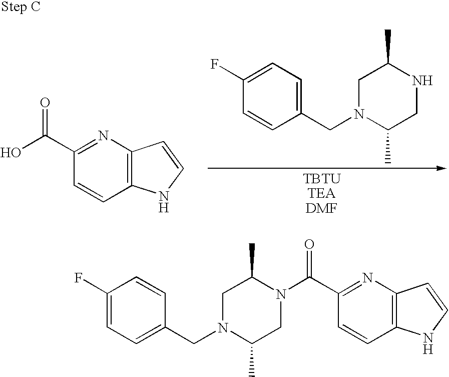

1H-pyrrolo[3,2-b]pyridine-5-carboxylic acid (0.49 g) and 1-[1-(4-fluoro-phenyl)-ethyl]-2S,5R-dimethyl-piperazine (0.67 g) were dissolved in dry DMF (5 mL) and TBTU (0.97 g) was added followed by triethylamine (1.3 mL). The mixture was stirred overnight, whereupon it was poured into water and the solid thus separated was filtered and dried. The crude material was purified by flash chromatography using 20% methanol: 80% dichloromethane as a solvent (Yield: 0.49 g, R_{f}: 0.74 min, Condition B, M+H⁺: 367).

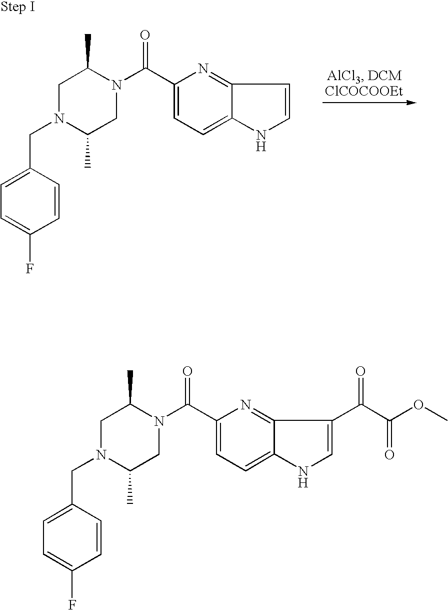

{4-[1-(4-Fluoro-phenyl)-ethyl]-2R,5S-dimethyl-piperazin-1-yl}-1H-pyrrolo[3,2-b]pyridin-5-yl)-methanone (0.35 g) was taken in dry dichloromethane (10 mL) and aluminum chloride (0.635 g) was added and the mixture was stirred under nitrogen for 2 h. To this was added methyl chlorooxoacetate (0.53 mL) and the stirring was continued for an additional 6 h and then quenched with methanol. The reaction mixture was concentrated and was purified by preparative chromatography (Yield: 0.11 g, R_{f}: 0.993 min, Condition B, M+H⁺: 453).

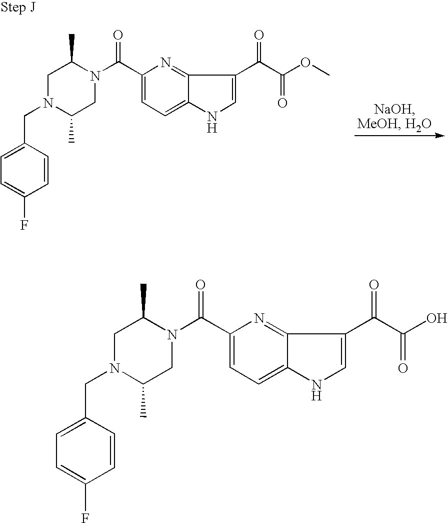

To the methanol (2 mL) and water (2 mL) suspension of the ester (0.11 g) was added sodium hydroxide (29.2 mg). The reaction was heated at 65° C. for 3 h. The reaction mixture was concentrated and the residue obtained was redissolved in water. The aqueous solution was acidified with concentrated hydrochloric acid to pH 2. The crude material was purified by preparative HPLC (Yield: 89 mg, R_{f}: 0.827 min, Condition B, M+H⁺: 439).

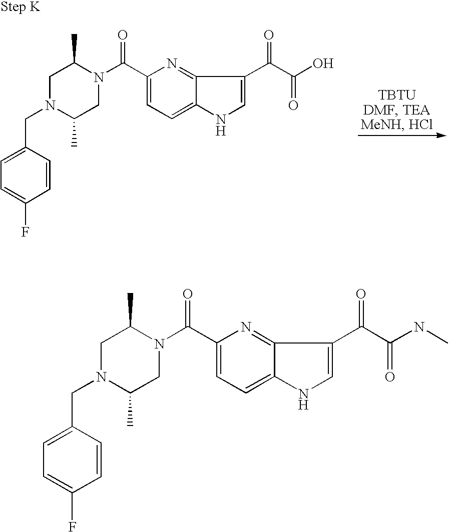

To the acid (84 mg) and methylamine hydrochloride (13 mg) dissolved in dry DMF (3 mL) was added TBTU (61.5 mg) followed by triethylamine (106.7 μl). The mixture was stirred for 5 h and was quenched with water. Extraction was done with ethyl acetate. The organic layer was separated, dried over sodium sulfate, filtered and concentrated. The crude material was purified by preparative HPLC (Yield: 21 mg, R_{f}: 0.90 min, Condition B, M+H⁺: 452).

### Example 12

### Preparation of 2-{6-Chloro-5-[4-(4-fluoro-benzyl)-2R,5S-dimethyl-piperazine-1-carbonyl]-1H-pyrrolo[3,2-b]pyridin-3-yl}-N-methyl-2-oxo-acetamide

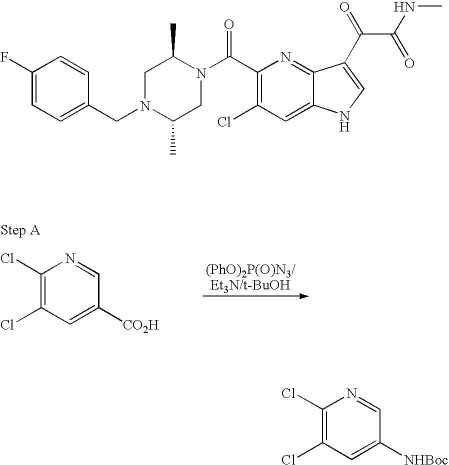

A mixture of 5,6-dichloronicotinic acid (5.0 g, 26.0 mmol), diphenylphosphoryl azide (7.1 g, 26.0 mmol), and triethylamine (2.63 g, 26.0 mmol) in t-butanol (50 mL) was heated at 80° C. for 18 h. The reaction mixture was cooled to room temperature and poured into ice water. The product was extracted with ethyl acetate. The extracts were washed with water, 10% sodium carbonate and brine and evaporated to give a brown oil which was purified on a silica gel column using hexane/ethyl acetate (1:1) to obtain the product (3.9 g, 58%) as a white solid.

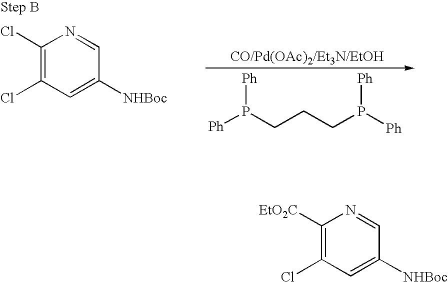

The above product (3.9 g, 14.8 mmol) was dissolved in EtOH (50 mL) and palladium acetate (2.6 g, 11.6 mmol), 1,3-bis(diphenylphosphino)propane (4.7 g, 6.8 mmol) and triethylamine (10.7 g, 106.6 mmol) were added sequentially and a stream of carbon monoxide was bubbled through the solution for 10 min. A balloon filled with CO was attached to the reaction flask and the mixture was stirred at 60° C. for 18 h. The mixture was cooled and diluted with ethyl acetate, filtered through a pad of Celite® and evaporated. Purification using silica gel chromatography using hexane/ethyl acetate provided 2.2 g of the product as a white solid.

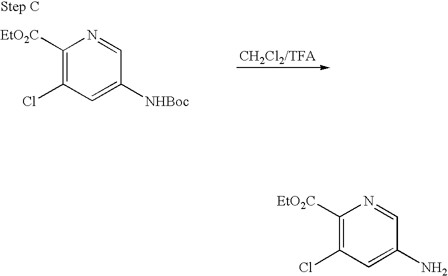

To a solution of 5-tert-butoxycarbonylamino-3-chloro-pyridine-2-carboxylic acid ethyl ester (3.0 g, 10.0 mmol) in methylene chloride (15 mL) was added 5 mL of trifluoroacetic acid. After stirring for 3 h the solvent was evaporated and the residue was dissolved in ethyl acetate and washed with 10% sodium carbonate solution, water and brine. The organic layer was dried with sodium sulfate and evaporated to yield an oil. Purification using silica gel chromatography with hexane/ethyl acetate gave ethyl 3-chloro-5-amino-2-pyridine carboxylate ethyl ester (1.3 g) as a white solid.

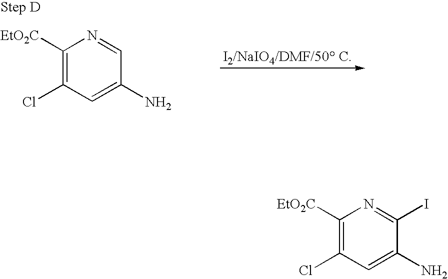

To a N,N-dimethylformamide (20 mL) solution of ethyl ethyl 3-chloro-5-amino-2-pyridine carboxylate (1.3 g, 6.5 mmol) was added iodine (1.6 g, 6.5 mmol)) and sodium metaperiodate (1.4, 6.5 mmol). The reaction was then heated to 60° C. for 18 h. After cooling to room temperature, the reaction was poured into a solution of sodium metabisulfite. The solid thus separated was filtered, washed with water and dried at high vacuum, yielding 1.6 g of the desired compound.

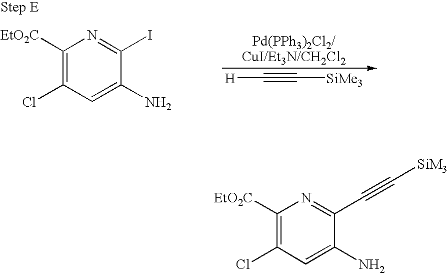

To a dichloromethane (25 mL) solution of ethyl 3-chloro-5-amino-6-iodo-2-pyridine carboxylate (1.6 g, 4.8 mmol) was added copper iodide (9.0 mg, 0.048 mmol), PdCl₂(PPh₃)₂ (17.0 mg, 0.024 mmol), triethylamine (1.0 ml, 7.2 mmol) and trimethylsilyl acetylene (1.0 ml, 7.3 mmol) sequentially. The reaction was stirred at room temperature for 4 h. The reaction mixture was concentrated and the residue was dissolved in ethyl acetate and water. The two layers were filtered through a Celite® pad. The ethyl acetate layer was separated, washed with water and brine then dried over Na₂SO₄. The solvent was evaporated and the residue was purified by silica gel chromatography using 30-100% ethyl acetate/hexane as solvent (Yield: 1.2 g).

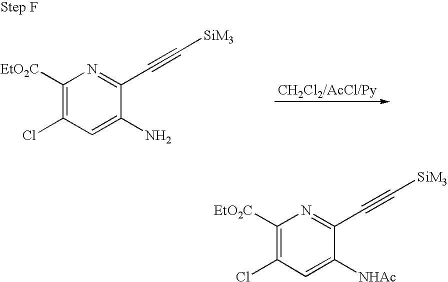

At 0° C., acetyl chloride (0.35 mL) was added to the dichloromethane (20 mL) solution of ethyl 3-chloro-5-amino-6-trimethylsilylacetylene-2-pyridine carboxylate (1.2 g) and pyridine (0.78 mL). The reaction was stirred at room temperature for 1 hr. The reaction was quenched with water and the dichloromethane layer was separated, dried over sodium sulfate and filtered. The filtrate was concentrated under vacuum. The residue was dried at high vacuum and was purified using silica gel chromatography with hexane/ethyl acetate (Yield: 1.3 g).

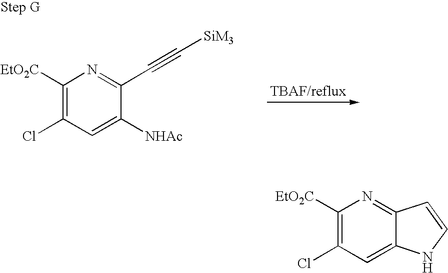

Tetrabutylammonium fluoride(11 mL, 1.0 M/THF) was added to the tetrahydrofuran (7 mL) solution of 3-chloro-5-acetylamino-6-trimethylsilanylethynyl-2-pyridine ethyl ester (1.3 g, 3.8 mmol). Reaction was stirred at 70° C. for 4 h. The reaction mixture was cooled to room temperature and was concentrated. The residue obtained was diluted with water. The solid thus separated was filtered and was washed with water. The solid was purified using silica gel chromatography with hexane/ethyl acetate. (Yield: 0.72 g).

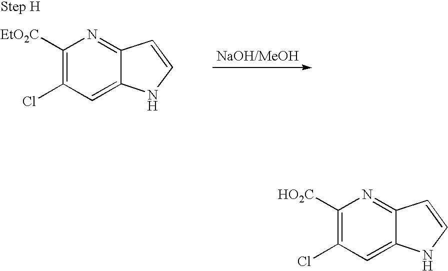

To the methanol (10 mL) and water (10 mL) suspension of 1H-pyrrolo[3,2-b]pyridine-6-chloro-5-carboxylic acid ethyl ester (0.7 g) was added sodium hydroxide (0.5 g). The reaction was heated at 50° C. for 2 h. The reaction mixture was concentrated and the residue obtained was redissolved in water. The aqueous solution was acidified with concentrated hydrochloric acid to pH 2. The solid thus separated was filtered, washed with water and dried under high vacuum. It was used as such for the next step without any purification (Yield: 0.69 g).

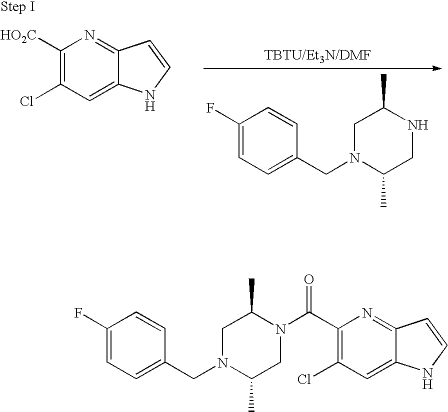

1H-pyrrolo[3,2-b]pyridine-6-chloro-5-carboxylic acid (0.6 g) and 1-[1-(4-fluoro-phenyl)-ethyl]-2S,5R-dimethyl-piperazine (0.67 g) were dissolved in dry DMF (5 mL) and TBTU (0.97 g) was added followed by triethylamine (1.3 mL). The mixture was stirred overnight at RT. The mixture was poured into water and the solid thus separated was filtered and dried. The crude material was purified by silica gel column chromatography using 20% methanol: 80% dichloromethane as solvents (Yield: 0.9 g).

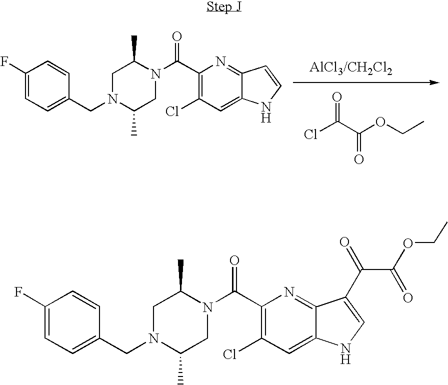

{4-[1-(4-Fluoro-phenyl)-ethyl]-2R,5S-dimethyl-piperazin-1-yl}-6-chloro-1H-pyrrolo[2,3-b]pyridin-5-yl)-methanone (0.3 g) was dissolved in dry dichloromethane (10 mL) and aluminum chloride (0.525 g) was added and the mixture was stirred under nitrogen for 2 h. To this, ethyl chlorooxoacetate (0.44 mL) was added and the stirring was continued for an additional 6 h and was then quenched with methanol. The reaction mixture was concentrated and was purified by preparative chromatography (Yield: 13 mg, R_{f}: 1.033 min, Condition B, M+H⁺: 487).

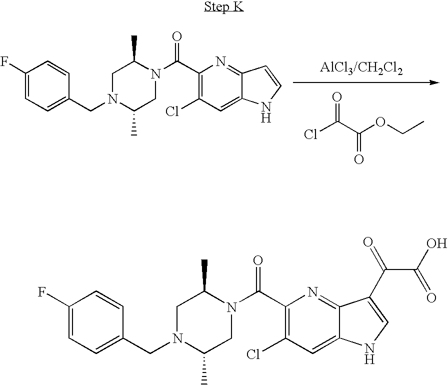

{4-[1-(4-Fluoro-phenyl)-ethyl]-2R,5S-dimethyl-piperazin-1-yl}-6-chloro-1H-pyrrolo[2,3-b]pyridin-5-yl)-methanone (0.6 g) was taken in dry dichloromethane (10 mL) and aluminum chloride (0.996 g) was added and the mixture was stirred under nitrogen for 2 h. To this, ethyl chlorooxoacetate (0.84 mL) was added and the reaction was continued for 10 days and was then quenched with ethanol. The reaction mixture was concentrated and the residue obtained was diluted with water and ethyl acetate. The organic layer was separated, dried over sodium sulfate, filtered and concentrated. The product stayed in the aqueous layer. The material in the aqueous layer was purified by preparative HPLC (Yield: 70 mg, R_{f}: 0.853 min, Condition B, M+H⁺: 473).

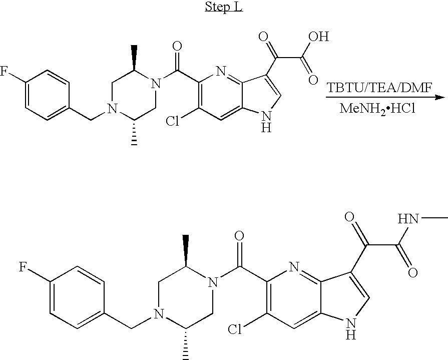

To {6-Chloro-5-[4-(4-fluoro-benzyl)-2R,5S-dimethyl-piperazine-1-carbonyl]-1H-pyrrolo[3,2-b]pyridin-3-yl}-oxo-acetic acid (70 mg) in 3 mL N,N-dimethylformamide was added 165 μl triethylamine, 95.2 mg TBTU and 20 mg methylamine hydrochloride. The reaction mixture was stirred at room temperature overnight. The reaction was quenched with water and was extracted with ethyl acetate. The organic layer was separated, dried over sodium sulfate, filtered and concentrated. The residue was purified by silica gel chromatography using 2% MeOH/dichloromethane to 10% MeOH/dichloromethane (Yield: 7.6 mg, R_{f}: 0.913 min, Condition B, M+H⁺: 486).

### Example 13

### Preparation of 2-{5-[4-(4-Fluoro-benzyl)-2R,5S-dimethyl-piperazine-1-carbonyl]-6-methoxy-1H-pyrrolo[3,2-b]pyridin-3-yl}-N-methyl-2-oxo-acetamide

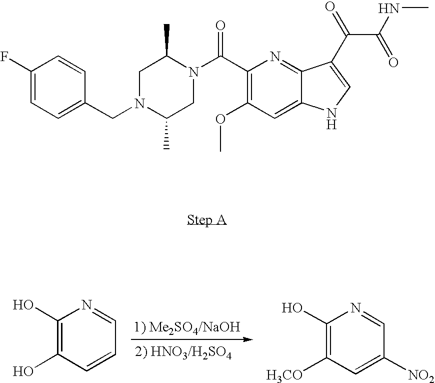

To NaOH (8.6 g, 0.215 mol) and dihydroxypyridine (22.0 g, 0.20 mol) in distilled water (75 mL) was added dimethylsulfate (25 g, 0.21 mol) at 5° C. dropwise and stirred at RT for 20 h. Then concentrated H₂SO₄ (50 mL) was added while cooling at 5° C. To this solution at 5° C. was added a cold solution of concentrated H₂SO₄ (20 mL) and concentrated HNO₃ (20 mL) while keeping the temperature at 10-15° C. After the addition the temperature was kept at 5° C. for 30 min then poured into ice water (400 mL). The dark brown solid was filtered out and dried (Yield: 3.3 g).

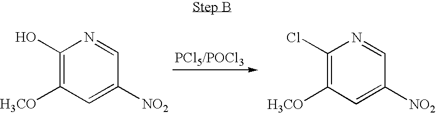

The above compound from Step A (3.3 g, 0.019 mol) was mixed with PCl₅ (3.0 g) and POCl₃ (23 mL) and refluxed for 2.5 h at 100° C. A dark green solution resulted which was cooled and the POCl₃ evaporated off. The mix was then poured into ice-water and the precipitate formed was filtered and washed with water and dried to provide 2.83 g of product as a white solid.

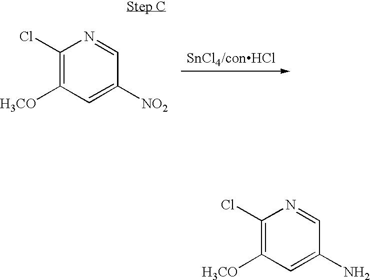

2-chloro-3-methoxy-5-nitropyridine (2.8 g, 14.9 mmol) was dissolved in concentrated HCl (30 mL) at 5° C., tin chloride (10.0 g) was added and stirred at 5° C. for 15 min and then heated at 80° C. for 1 h. The reaction mixture was cooled and neutralized with 20% NaOH until pH 8, extracted with EtOAc, washed with water and dried with sodium sulfate and evaporated to obtain a brown solid (1.83 g, 78%).

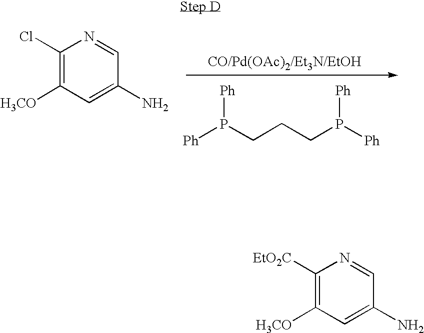

The 2-chloro-3-methoxy-5-aminopyridine (1.83 g, 11.58 mmol) was dissolved in EtOH (50 mL) and palladium acetate (2.0 g, 9.0 mmol), 1,3-bis(diphenylphosphino)propane (3.5 g, 5.3 mmol), triethylamine (8.4 g, 83.38 mmol) were added sequentially and a stream of carbon monoxide was bubbled through the solution for 10 min. A balloon filled with CO was attached to the reaction flask and the mixture was stirred at 60° C. for 18 h. The mixture was cooled and diluted with ethyl acetate, filtered through a pad of Celite® and evaporated. Purification using silica gel chromatography using hexane/ethyl acetate mixture provided 310 mg of product.

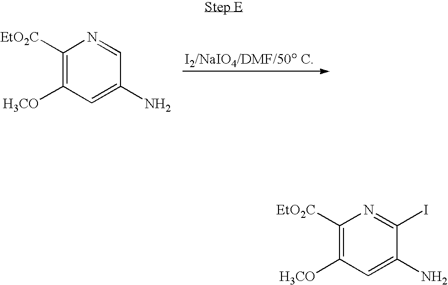

To a N,N-dimethylformamide (10 mL) solution of ethyl 3-methoxy-5-aminopyridine-2-carboxylate (300 mg, 1.53 mmol) was added iodine (388 mg, 1.53 mmol)) and sodium metaperiodate (327 mg, 1.53 mmol). The reaction was then heated to 60° C. for 18 h. After cooling to room temperature, the reaction was poured into a solution of sodium metabisulfite. The solid thus separated was filtered, washed with water and dried under high vacuum (Yield: 350 mg).

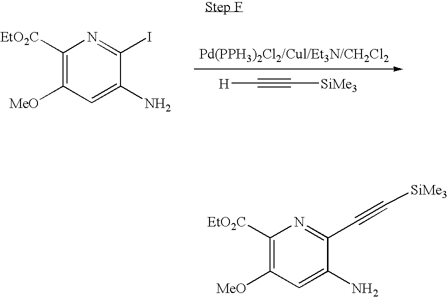

To the dichloromethane (5 mL) solution of ethyl 3-methoxy-5-amino-6-iodopyridine-2-carboxylate (350 mg, 1.08 mmol) was added copper iodide (2.01 mg, 0.0108 mmol), PdCl₂(PPh₃)₂ (3.8 mg, 0.0054 mmol), triethylamine (162 mg, 1.6 mmol) and trimethylsilyl acetylene (160 mg, 1.6 mmol) sequentially. The reaction was stirred at room temperature for 4 h. The reaction mixture was concentrated and the residue was dissolved in ethyl acetate and water. The two layers were filtered through a Celite® pad. The ethyl acetate layer was separated, washed with water and brine then dried over Na₂SO₄. The solvent was evaporated and the residue was purified by silica gel chromatography using 30-100% ethyl acetate/hexane as solvent (Yield: 320 mg).

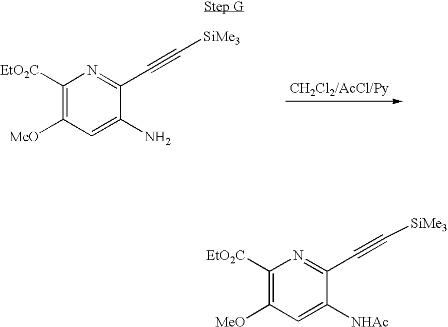

At 0° C., acetyl chloride (0.0.086 mL) was added to the dichloromethane (5 mL) solution of ethyl 3-methoxy-5-amino-6-trimethylsilylacetylenepyridine-2-carboxylate (320 mg) and pyridine (0. 176 mL). The reaction was stirred at room temperature for 1 h and then quenched with water. The dichloromethane layer was separated, dried over sodium sulfate and filtered. The filtrate was concentrated under vacuum. The residue obtained was dried under high vacuum and was purified using silica gel column chromatography with hexane/ethyl acetate mixture (Yield: 290 mg).

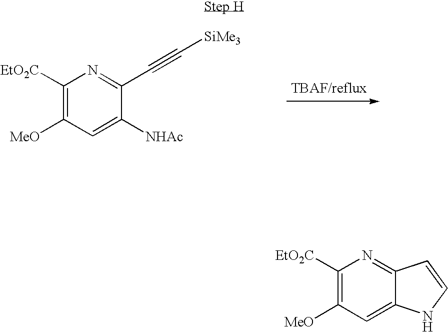

Tetrabutylammonium fluoride (3 mL, 1.0 M in THF) was added to 3-methoxy-5-acetylamino-6-trimethylsilanylethynyl-2-pyridine ethyl ester (290 mg, 0.86 mmol). The reaction was stirred at 70° C. for 4 h. The reaction mixture was cooled to room temperature and concentrated. The residue obtained was diluted with water. The solid thus separated was filtered and was washed with water. The solid was purified using silica gel column chromatography with hexane/ethyl acetate mixture (Yield: 97 mg).

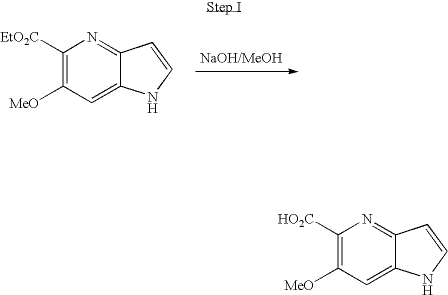

To the methanol (3 mL) and water (3 mL) suspension of 1H-pyrrolo[3,2-b]pyridine-6-methoxy-5-carboxylic acid ethyl ester (97.0 mg) was added sodium hydroxide(70.0 mg). The reaction was heated at 50° C. for 2 h. The reaction mixture was concentrated and the residue obtained was redissolved in water. The aqueous solution was acidified with concentrated hydrochloric acid to pH 2. The solid thus separated was filtered, washed with water and dried under high vacuum. The material was used as such for the next step without any purification (Yield: 84 mg).

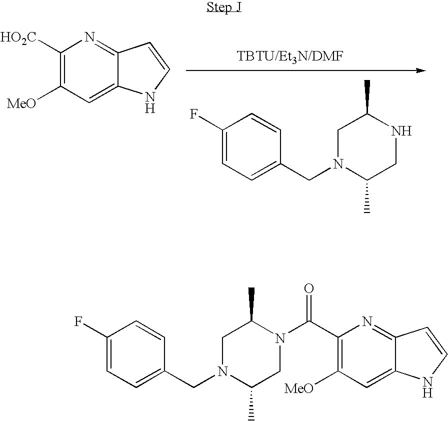

1H-pyrrolo[3,2-b]pyridine-6-methoxy-5-carboxylic acid (84 mg) and 1-[1-(4-fluoro-phenyl)-ethyl]-2S,5R-dimethyl-piperazine (98 mg) were dissolved in dry DMF (5 mL) and TBTU (141 mg) was added followed by triethylamine (0.184 ml). The mixture was allowed to stir overnight. It was then poured into water and the solid thus separated was filtered and dried. The crude material was purified by silica gel column chromatography using 20% methanol: 80% dichloromethane as a solvent (Yield: 96 mg, R_{f}: 0.82 min, Condition B, M+H⁺: 397).

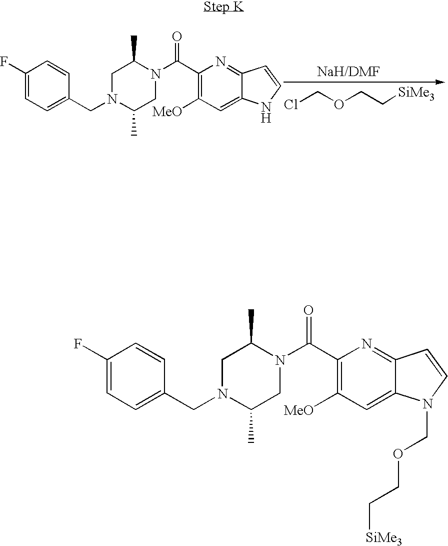

To a solution of the product in Step J (55 mg, 0.14 mmol) in DMF (3 mL) was added NaH (0.15 mmol). The reaction was stirred at RT for 10 min whereupon 2-[trimethylsilyl]ethoxymethylchloride (24 mg, 0.15 mmol) was added and the mixture was stirred for 4 h. The reaction mixture was quenched with water and extracted with ethyl acetate, dried and evaporated to give 65 mg of product (R_{f}: 1.50 min, Condition B, M+H⁺: 527).

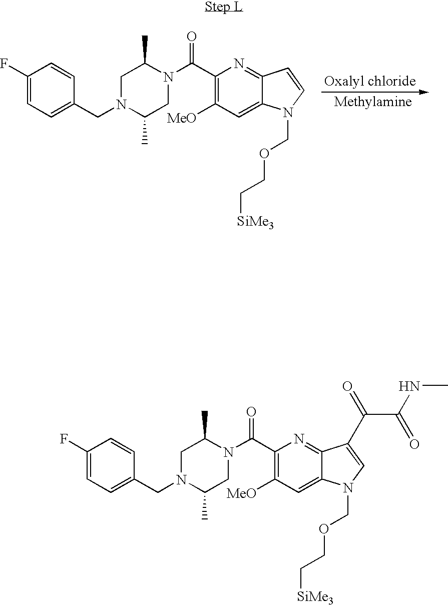

The product from step K (20 mg, 1.0 equiv.) was treated with 2 M oxalyl chloride in methylene chloride (6.0 equiv.) and allowed to stir overnight. The volatiles were then evaporated and the residue dried under vacuum. The residue was dissolved in THF and treated with excess methylamine in THF. Upon complete reaction, the mixture was concentrated and the product was purified by preparative TLC to obtain 6 mg of the desired compound.

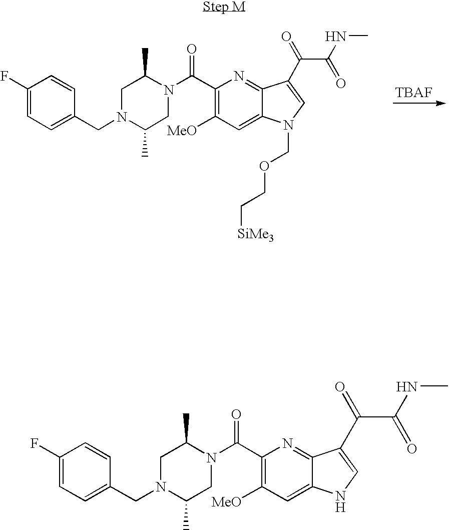

The product from Step L (6.0 mg) was dissolved in 3 mL of a 1 M solution of TBAF in THF and heated at 80° C. for 4 h. The reaction mixture was cooled to room temperature and was concentrated. The residue obtained was diluted with water. The solid thus separated was filtered and washed with water. The product was purified by preparative TLC and 2 mg was obtained (R_{f}: 0.87 min, Condition B, M+H⁺: 482).

### Example 14

### Preparation of [4-(4-Fluoro-benzyl)-2R,5S-dimethyl-piperazin-1-yl]-(3-methoxy-5H-pyrrolo[2,3-b]pyrazin-2-yl)-methanone

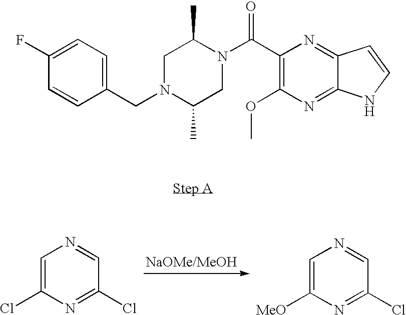

To a solution of 2,6-dichloropyrazine (5.0 g) in methanol (30 mL) was added sodium methoxide (2 eq.) and the reaction was refluxed overnight. To this was added additional NaOMe (3 eq.) and the reaction mixture was refluxed for 8 h. The reaction mixture was cooled to room temperature and concentrated. The residue obtained was washed with water and was extracted with ethyl acetate. The organic layer was separated, dried over sodium sulfate, filtered and concentrated. The oil obtained was placed under high vacuum overnight and was used as such for the next step (Yield: 3 g, R_{f}: 1.007 min, Condition B, M+41: 186).

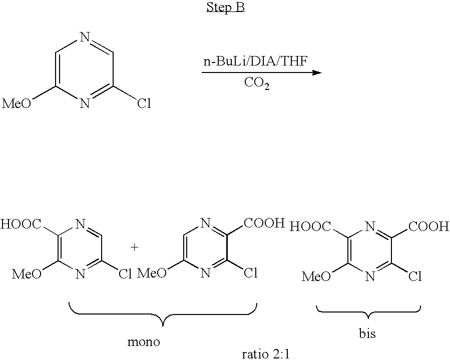

To a 100 mL oven-dried flask, anhydrous THF (40 mL) was added and was cooled to −78° C. under argon. To this n-BuLi (2.5 M in hexanes, 3.05 mL) was added dropwise at −78° C. After 15 min diisopropylamine (1.07 mL) was added dropwise. The reaction was stirred at −78° C. for 1 h and to this was added a solution of 2-chloro-6-methoxy-pyrazine (0.5 g) in THF (5 mL) dropwise at −78° C. Stirring was continued for another 1 h at −78° C. followed by quenching with CO₂ gas. The reaction was then stirred at room temperature for 1 h, whereupon it was quenched with conc. HCl and extracted with ethyl acetate. The organic layer was extracted with a 1N NaOH solution. The basic solution was neutralized with conc. HCl and the aqueous layer was extracted with ethyl acetate. The organic layer was separated, dried over sodium sulfate, filtered and concentrated. The residue obtained was dried under high vacuum and was used as such for the next step (Yield: 0.3 g, R_{f}: 0.767 min, Condition B, M+H⁺: 189).

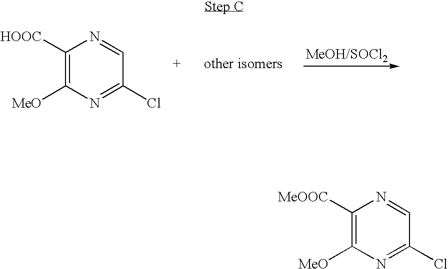

To a solution of 2-chloro-6-methoxy-4-pyrazine carboxylic acid (0.3 g) in MeOH (5 mL) was added thionyl chloride (1 mL) and reaction mixture was refluxed for 1 h. The reaction was cooled to room temperature and was concentrated. The residue obtained was washed with saturated NaHCO₃ solution and was extracted with ethyl acetate. The organic layer was separated, dried over sodium sulfate, filtered and concentrated. The residue obtained was purified by SiO₂ chromatography using 10% ethyl acetate: hexane to 30% ethyl acetate: hexane (Yield: 83 mg, R_{f}: 1.043 min, Condition B, M+H⁺: 203).

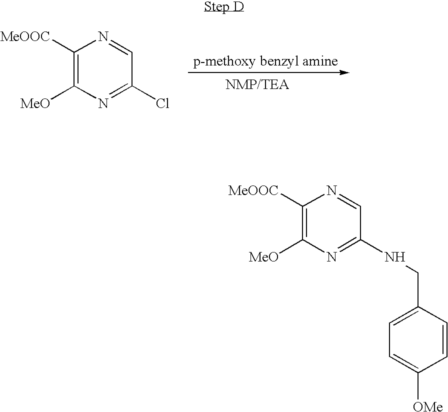

To a solution of 2-chloro-6-methoxy-pyrazine-4-methyl ester (83 mg) in NMP (2 mL) was added 64 μl of 4-methoxy benzyl amine and 61.7 μl of TEA. The reaction was stirred at 70° C. for 4 h. The reaction was cooled to room temperature and was quenched with water and ethyl acetate. The organic layer was separated, dried over sodium sulfate, filtered and concentrated. The residue obtained was purified by SiO₂ chromatography using 50% ethyl acetate: hexane to 100% ethyl acetate (Yield: 70 mg, R_{f}: 1.253 min, Condition B, M+H⁺: 304).

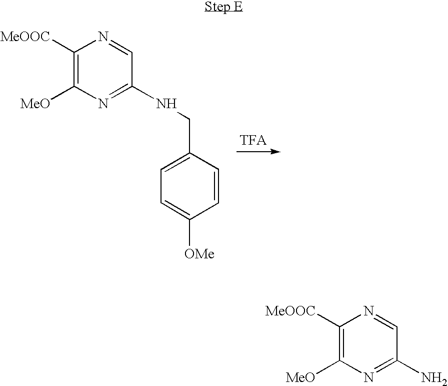

To a 10 mL flask was added 2-(4-methoxy benzyl amine)-6-methoxy-pyrazine 4-methyl ester (0.12 g) and of TFA (2 mL). The reaction was stirred at 40° C. for 5 h. The reaction was concentrated and the residue obtained was triturated with water. The solid thus separated was filtered and dried under high vacuum (Yield: 95 mg, R_{f}: 0.56 min, Condition B, M+H⁺: 184).

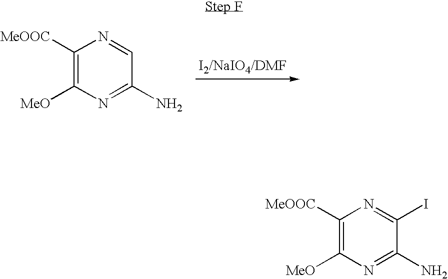

To a N,N-dimethylformamide (3 mL) solution of 2-amino-6-methoxy-pyrazine-4-methyl ester (95 mg) was added iodine (105.4 mg) and sodium metaperiodate (44.4 mg). The reaction was then heated to 60° C. for 24 h. After cooling to room temperature, the reaction was poured into a solution of sodium metabisulfite. The solid thus separated was filtered, washed with water and dried under high vacuum (Yield: 108 mg, R_{f}: 0.833 min, Condition B, M+H⁺: 310).

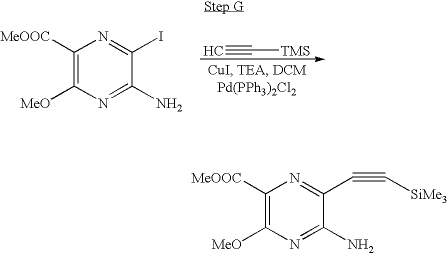

To the dichloromethane (4 mL) solution of 2-amino-3-iodo-6-methoxy pyrazine-4-methyl ester (108 mg) was added copper iodide (4.7 mg), PdCl₂(PPh₃)₂ (2.4 mg), triethylamine (73 μl) and trimethylsilyl acetylene (54.3 μl) sequentially. The reaction was stirred at room temperature for 4 h. The reaction mixture was concentrated and the residue was dissolved in ethyl acetate and water. The ethyl acetate layer was separated, washed with water and brine then dried over Na₂SO₄. The solvent was evaporated and the residue was purified by SiO₂ chromatography using 15-50% ethyl acetate/hexane as a solvent (Yield: 50 mg, R_{f}: 1.523 min, Condition B, M+H⁺: 280).

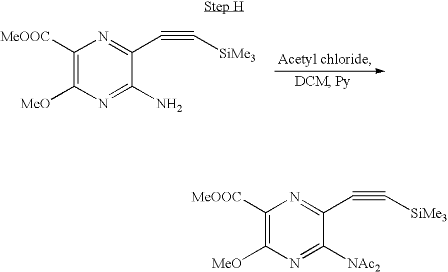

At 0° C. was added acetyl chloride (15 μl) to the dichloromethane (4 mL) solution of 2-amino-3-trimethylsilylacetylene-6-methoxy-pyrazine-4-methyl ester (50 mg) and pyridine (29 μl). The reaction was stirred at room temperature for 8 h. At this point, to the reaction mixture was added another 29 μl of pyridine and 15 μl of acetyl chloride. The reaction mixture was then stirred at room temperature overnight. The reaction was then quenched with water and the dichloromethane layer was separated, dried over sodium sulfate and filtered. The filtrate was concentrated under vacuum and the residue obtained was dried under high vacuum and used as such for the next step without any purification (Yield: 60 mg, R_{f}: 1.653 min, Condition B, M+H⁺: 364).

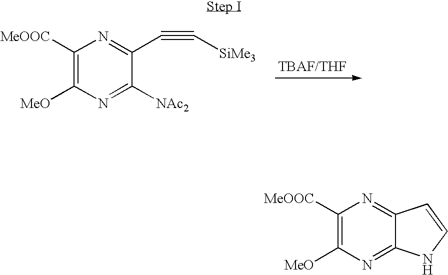

Tetrabutylammonium fluoride (2 mL, 1.0 M in THF) was added to the tetrahydrofuran (1 mL) solution of 2-acetylamino-3-trimethylsilanylethynyl-6-methoxy-pyrazine-4-methyl ester (60 mg). The reaction was stirred at 70° C. for 2 h and then cooled to room temperature and concentrated. The residue obtained was diluted with water and was extracted with ethyl acetate. The organic layer was separated, dried over sodium sulfate, filtered and concentrated. The residue obtained was dried under high vacuum and was used as such for the next step without any purification (Yield: 40 mg, R_{f}: 0.773 min, Condition B, M+H⁺: 208).

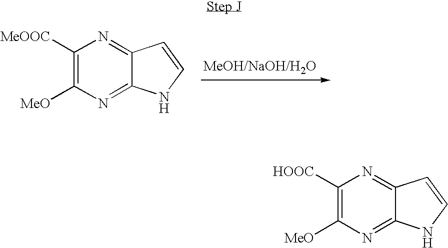

To the methanol (3 mL) and water (3 mL) suspension of 6-methoxy-4,7-diazaindole-5-methyl ester (40 mg) was added sodium hydroxide (3 mg). The reaction was heated at 60° C. for 1 h. The reaction mixture was concentrated and the residue obtained was redissolved in water. The aqueous solution was acidified with conc. HCl to pH 2. The solid thus separated was filtered, washed with water and dried under high vacuum. It was used as such for the next step without any purification (Yield: 26 mg, R_{f}: 0.547 min, Condition B, M+H⁺: 194).

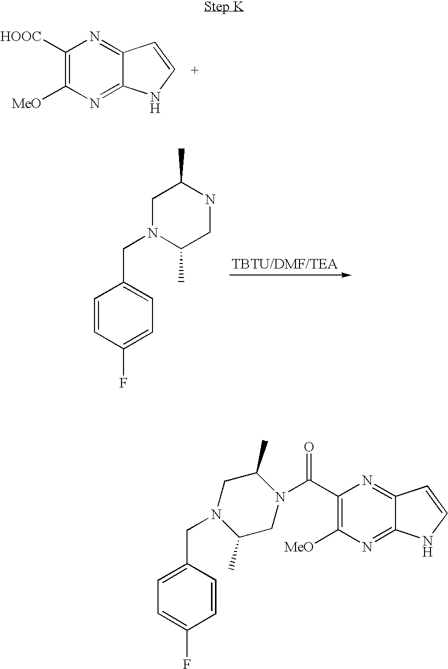

6-Methoxy-4,7-diazaindole-5-carboxylic acid (26 mg) and 1-[1-(4-fluoro-phenyl)-methyl]-2S,5R-dimethyl-piperazine (30 mg) were dissolved in dry DMF (3 mL) and TBTU (43.2 mg) was added followed by triethylamine (56 μl). The mixture was stirred for 5 days and was then poured into water and the solid thus separated was filtered and dried. The crude material was purified by radial chromatography using ethyl acetate as a solvent (Yield: 6 mg, R_{f}: 0.887 min, Condition B, M+H⁺: 398).

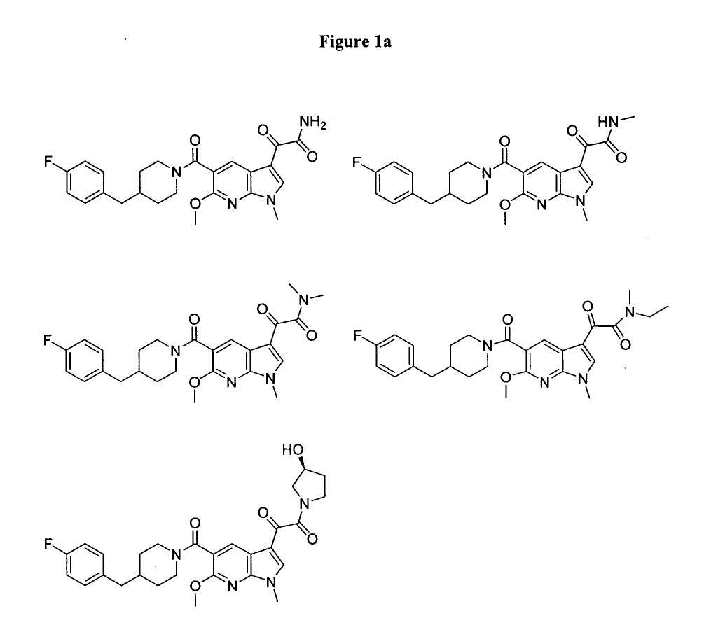

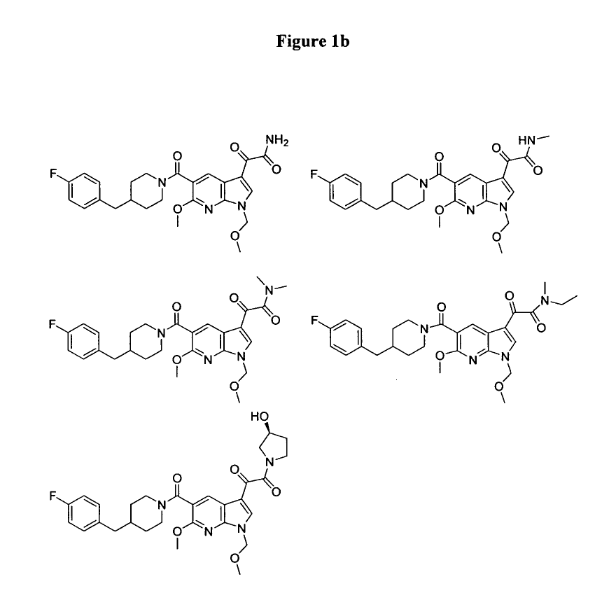

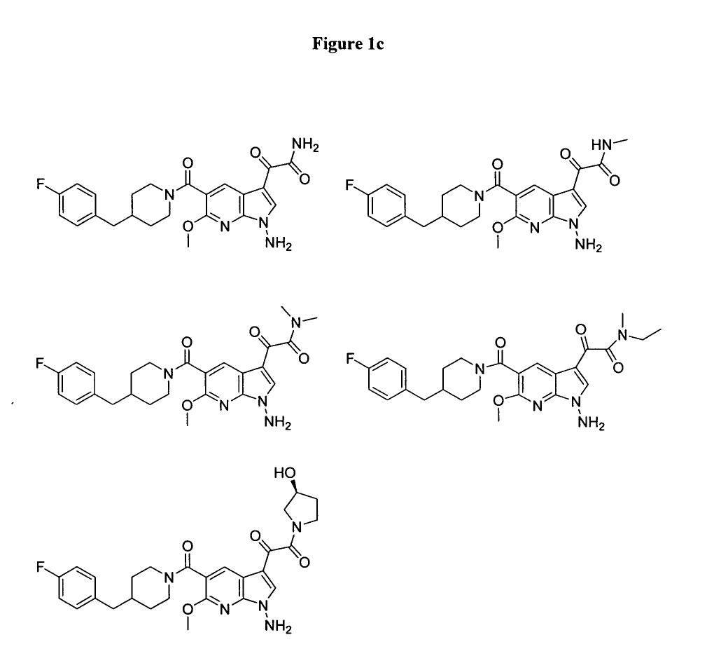

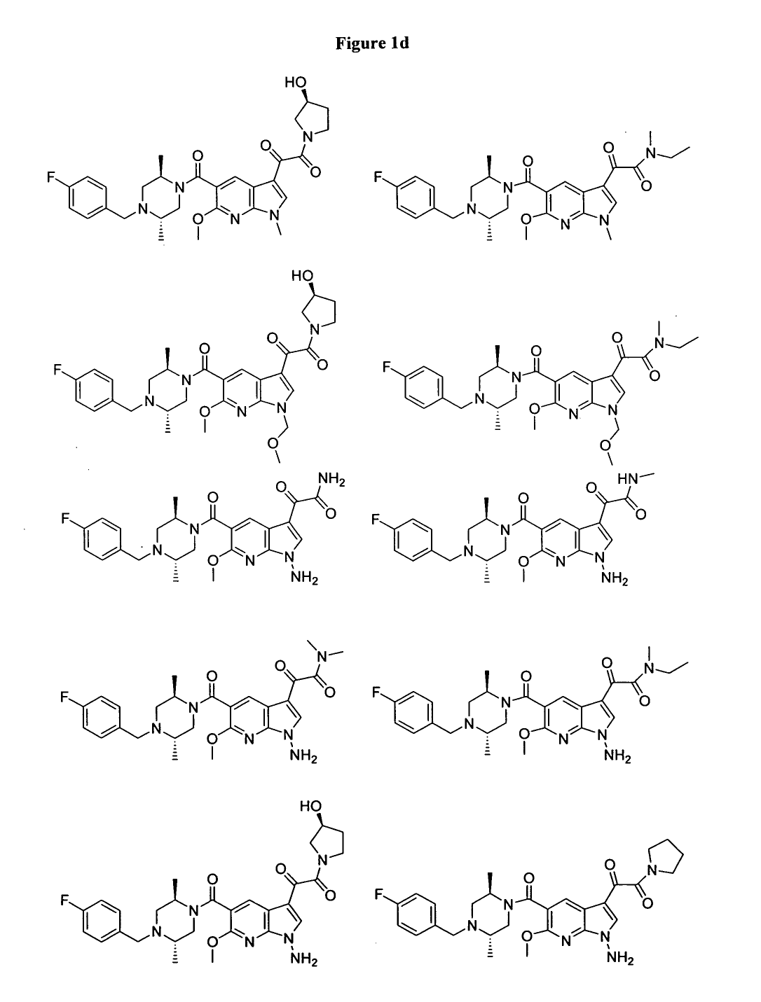

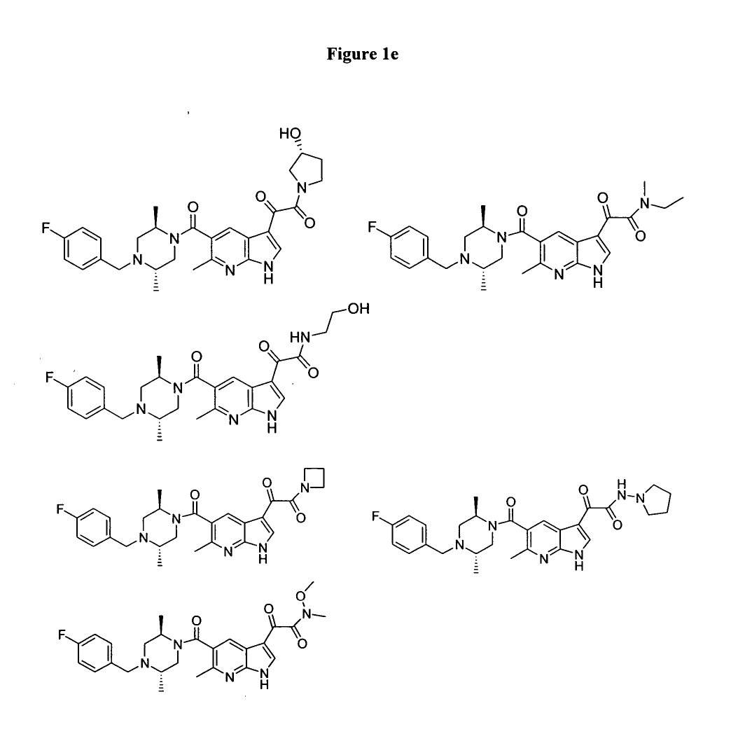

Biological activity and structural data for compounds made by the methods described herein are included in Table 1. Additional compounds of the invention which can be made by the foregoing methods include, but are obviously not limited to, those shown in FIGS. 1a-1e.

### Example 15

### Biological Activity

The activity of the compounds of the invention can be determined with the in vitro assay described below. Table 1 shows the activity data for a number of such compounds.

Assay for p38 Kinase Inhibition—p38α Flash Plate Assay

The compounds to be tested were solubilized in DMSO and diluted with water to the desired concentrations. The p38 kinase was diluted to 20 nM into a buffer containing 20 mM MOPS, pH 7.0, 25 mM beta-glycerol phosphate, 2 mg/ml gelatin, 0.5 mM EGTA (ethylene-bis-(oxyethylenenitrilo)-tetraacetic acid), and 4 mM dithiothreitol (DTT).

The reaction was carried out by mixing 20 μl test compound with 10 μl of a substrate cocktail containing 0.2 mM biotinylated peptide substrate and 0.6 mM ATP (+100 μCi/ml gamma-³³P-ATP) in a 5× assay buffer. The reaction was initiated by the addition of 10 μl of p38 kinase. Final assay conditions were 25 mM MOPS, pH 7.0, 26.25 mM beta-glycerol phosphate, 80 mM KCl, 22 mM MgCl₂, 3 mM MgSO₄, 1 mg/ml gelatin, 0.625 mM EGTA, 1 mM DTT, 0.05 mM peptide substrate, 150 μM ATP, and 5 nM enzyme. After a 60 minute incubation at 30° Celsius, the reaction was stopped by the addition of 10 μl per reaction of 0.25 M phosphoric acid.

A portion of the reaction was transferred to a streptavidin-coated Flash Plate (Perkin Elmer); the Flash Plate was incubated for 60 minutes at 30° Celsius and then washed 3× with PBS containing 0.01% Tween-20.

Counts incorporated are determined on a scintillation counter. Relative enzyme activity was calculated by subtracting background counts (counts measured in the absence of enzyme) from each result, and comparing the resulting counts to those obtained in the absence of inhibitor. IC₅₀ values were determined with curve-fitting plots available with common software packages. The IC₅₀ was expressed as the concentration of compound which inhibited the enzyme activity by 50%.

The compounds of the invention exhibit varying levels of activity towards p38α kinase. Table 1 provides in vitro activity data generated using the assay described above.

TABLE 1

| | | P38 α- | HPLC | Retention | Mass |
|---|---|---|---|---|---|
| | | FLASHPLATE | Column | Time | Observed |
| No. | Structure | IC50 (μM) | Conditions | (min) | (M + H)+ |
| 1 | 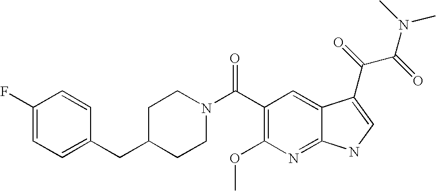 | 0.060 | | | 466 |
| 2 | 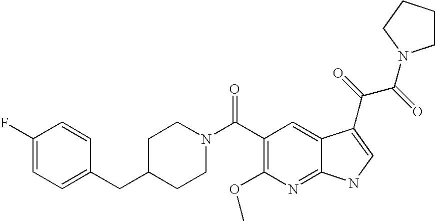 | 0.050 | A | 5.96 | 493 |
| 3 | 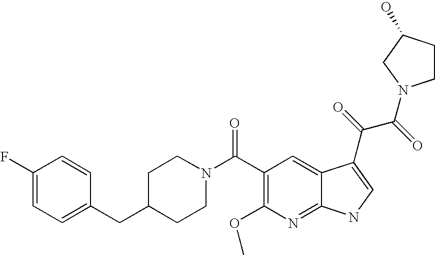 | 0.032 | A | 2.540 | 509 |
| 4 | 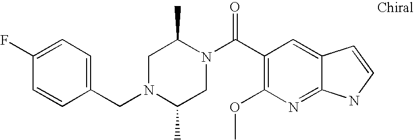 | 0.035 | A | 2.47 | 397 |
| 5 | 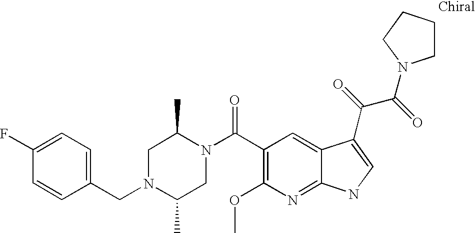 | 0.03 | A | 2.167 | 522 |
| 6 | 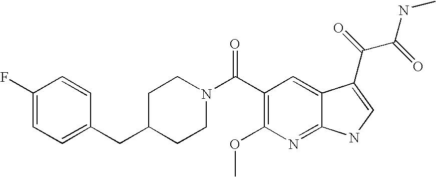 | 0.044 | A | 3.347 | 453 |
| 7 | 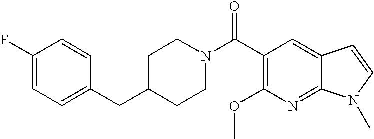 | 0.16 | A | 4.37 | 382 |
| 8 | 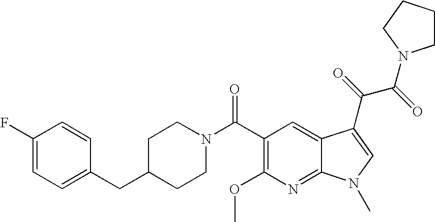 | 0.035 | A | 4.06 | 507 |
| 9 | 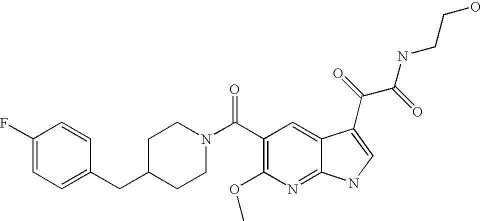 | 0.056 | A | 2.740 | 483 |
| 10 | 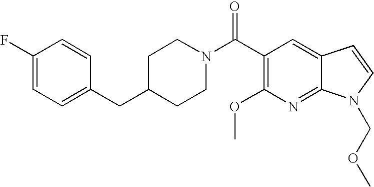 | 0.34 | A | 4.25 | 411 |
| 11 | 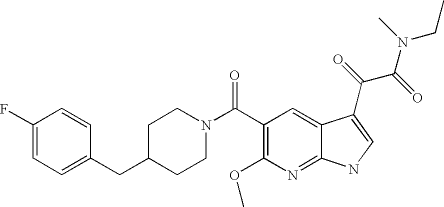 | 0.063 | A | 3.140 | 481 |
| 12 | 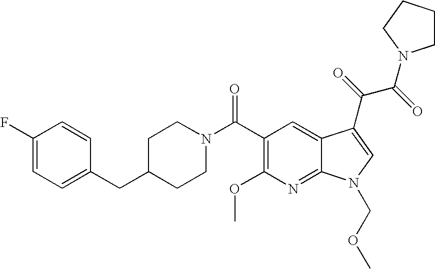 | 0.079 | A | 3.58 | 538 |
| 13 | 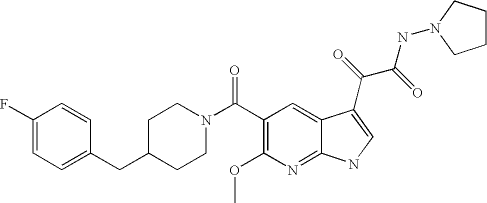 | 0.039 | A | 3.34 | 508 |
| 14 | 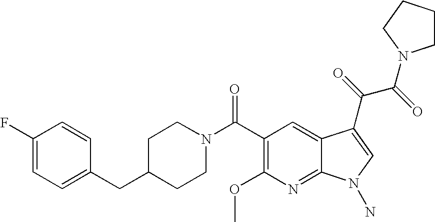 | 0.030 | A | 3.127 | 508 |
| 15 | 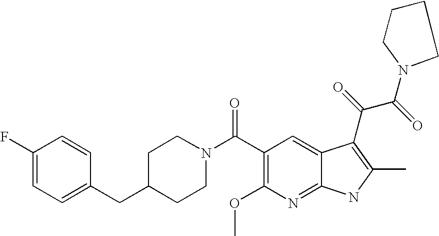 | 0.038 | A | 3.19 | 507 |
| 16 | 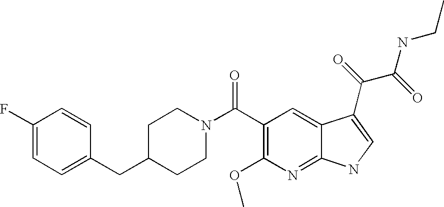 | 0.039 | A | 3.300 | 467 |
| 17 | 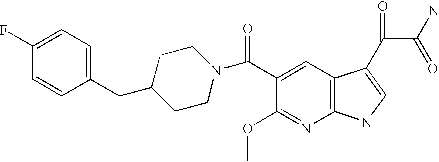 | 0.057 | A | 3.3 | 439 |
| 18 | 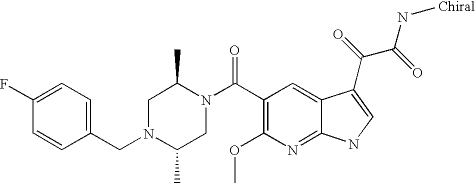 | 0.02 | A | 1.847 | 482 |
| 19 | 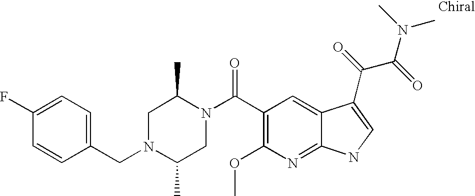 | 0.0082 | A | 1.833 | 496 |
| 20 | 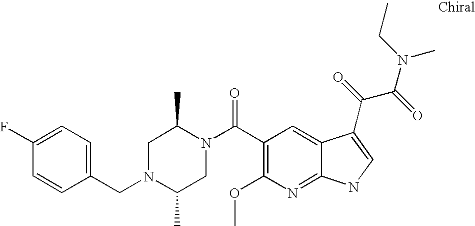 | 0.012 | A | 1.94 | 510 |
| 21 | 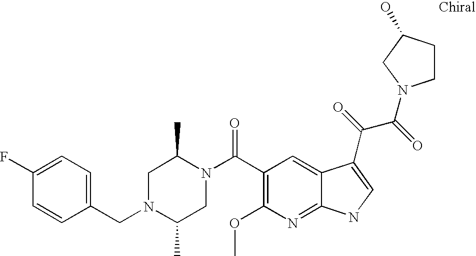 | 0.01 | A | 1.68 | 538 |
| 22 | 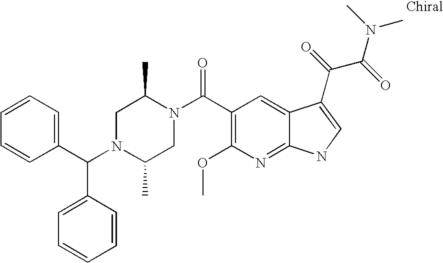 | 0.07 | A | 2.467 | 553 |
| 23 | 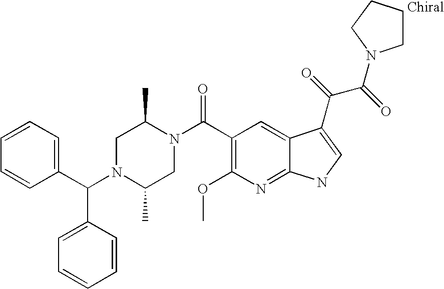 | 0.12 | A | 2.607 | 579 |
| 24 | 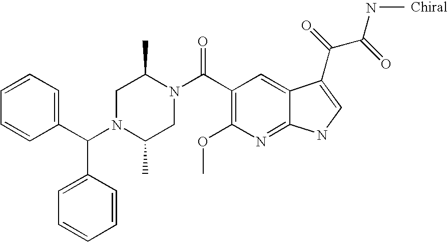 | 0.042 | A | 2.487 | 539 |
| 25 | 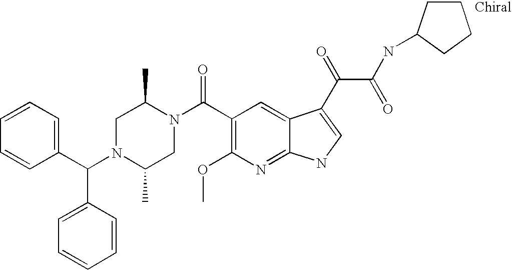 | 0.13 | A | 3.067 | 593 |
| 26 | 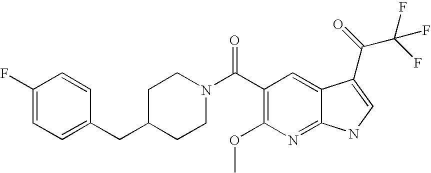 | 0.095 | A | 4 | 464 |
| 27 | 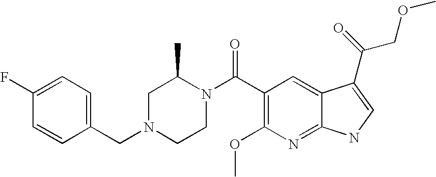 | 0.076 | A | 3.1 | 435 |
| 28 | 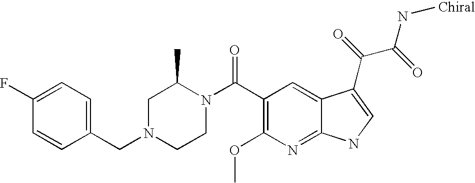 | | A | 1.747 | 468 |
| 29 | 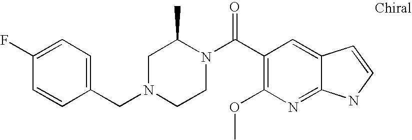 | | A | 1.81 | 419 |
| 30 | 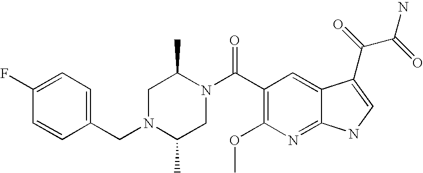 | 0.01 | A | 1.76 | 468 |
| 31 | 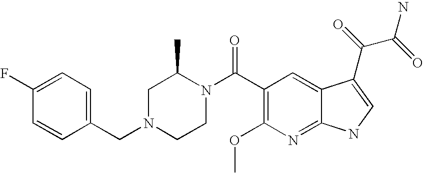 | 0.1 | A | 1.707 | 454 |
| 32 | 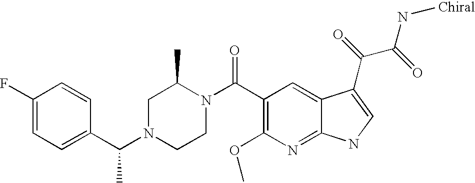 | 0.03 | B | 1.887 | 483 |
| 33 | 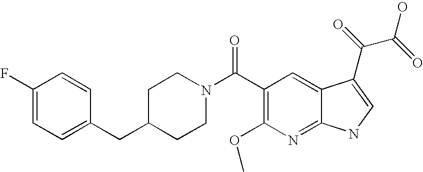 | 0.015 | A | 3.153 | 440 |
| 34 | 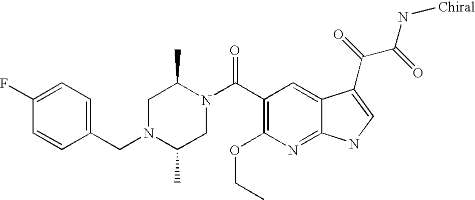 | 0.03 | A | 1.953 | 496 |
| 35 | 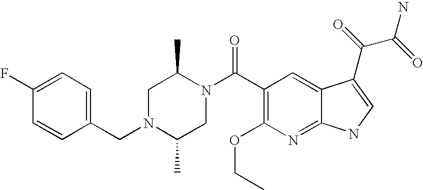 | 0.02 | A | 1.927 | 483 |
| 36 | 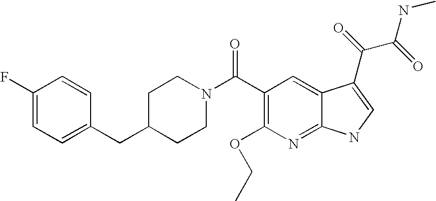 | 0.064 | A | 3.340 | 467 |
| 37 | 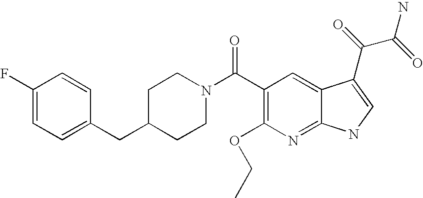 | 0.067 | A | 3.600 | 454 |
| 38 | 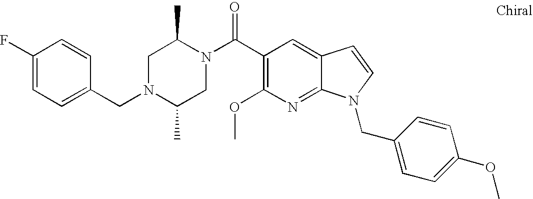 | 1.5 | A | 3.72 | 517.2 |
| 39 | 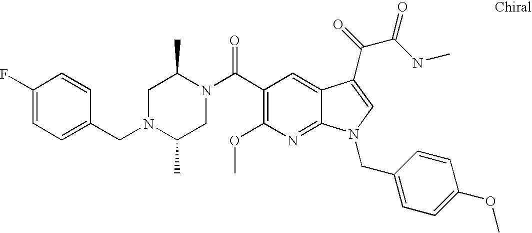 | 0.61 | A | 2.7 | 601 |
| 40 | 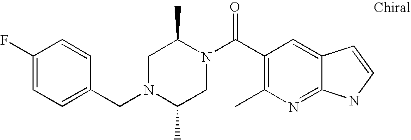 | 0.42 | B | 0.767 | 380 |
| 41 | 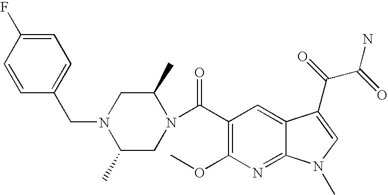 | 0.02 | B | 1.047 | 482 |
| 42 | 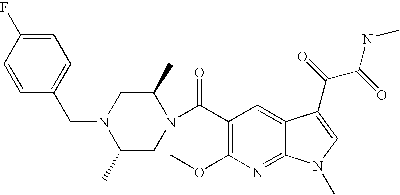 | 0.02 | B | 1.107 | 496 |
| 43 | 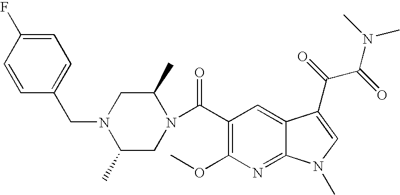 | 0.01 | B | 1.053 | 510 |
| 44 | 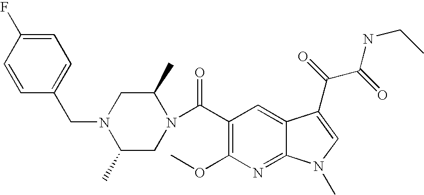 | 0.07 | B | 1.17 | 510 |
| 45 | 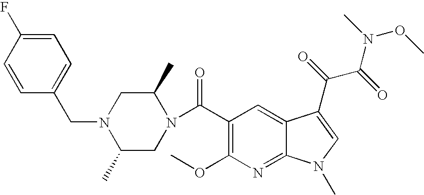 | 0.02 | B | 1.127 | 526 |
| 46 | 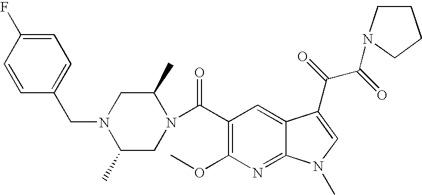 | 0.05 | B | 1.14 | 536 |
| 47 | 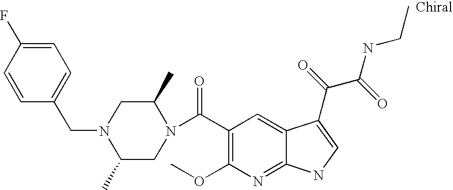 | 0.02 | B | 1.03 | 496 |
| 48 | 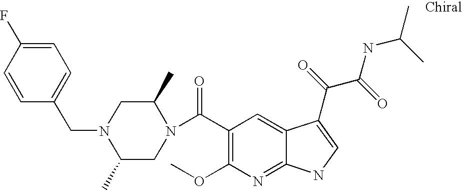 | 0.05 | B | 1.093 | 510 |
| 49 | 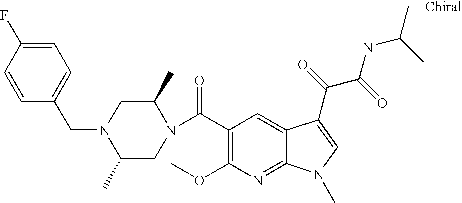 | 0.06 | B | 1.287 | 524 |
| 50 | 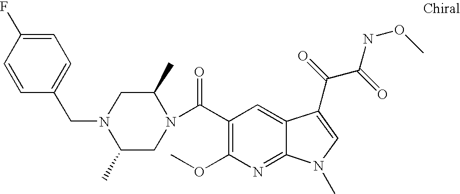 | 0.04 | B | 1.10 | 512 |
| 51 | 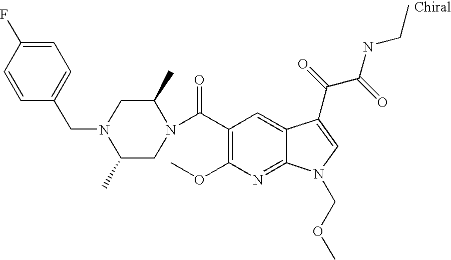 | 0.06 | B | 1.18 | 540 |
| 52 | 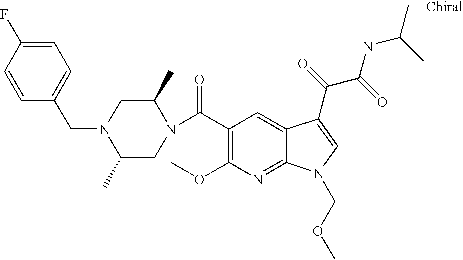 | 0.06 | B | 1.28 | 564 |
| 53 | 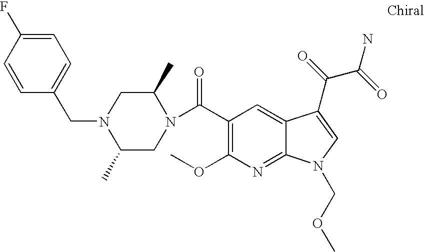 | 0.03 | B | 1.053 | 512 |
| 54 | 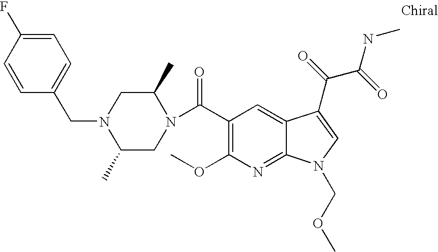 | 0.05 | B | 1.107 | 526 |
| 55 | 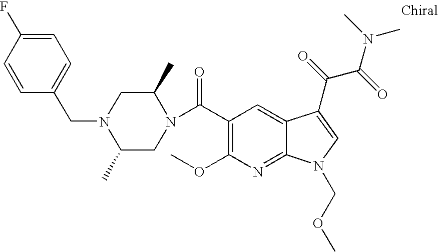 | 0.02 | B | 1.08 | 540 |
| 56 | 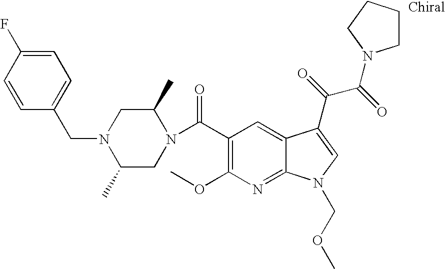 | 0.04 | B | 1.17 | 566 |
| 57 | 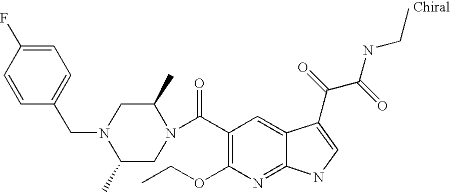 | 0.03 | B | 1.07 | 510 |
| 58 | 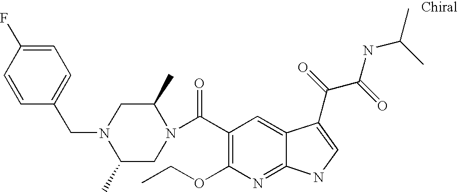 | 0.05 | B | 1.127 | 524 |
| 59 | 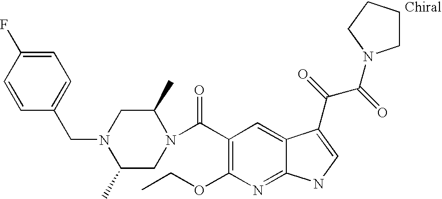 | 0.03 | B | 1.067 | 536 |
| 60 | 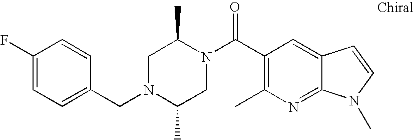 | 0.16 | B | 0.867 | 395 |
| 61 | 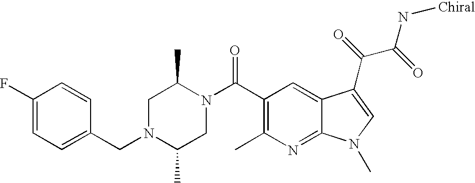 | 0.09 | B | 0.973 | 480 |
| 62 | 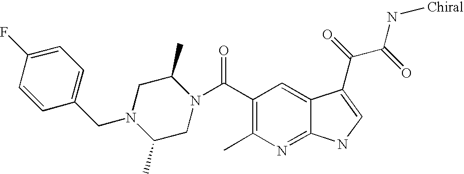 | 0.07 | B | 0.887 | 466 |
| 63 | 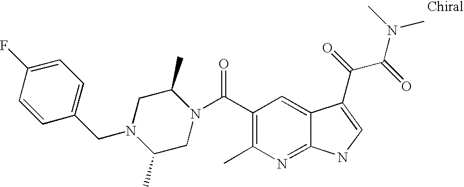 | 0.03 | B | 0.93 | 481 |
| 64 | 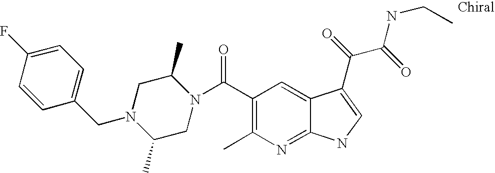 | 0.08 | B | 0.96 | 481 |
| 65 | 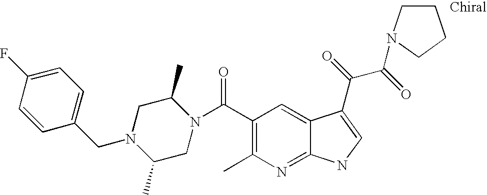 | 0.08 | B | 1.00 | 507 |
| 66 | 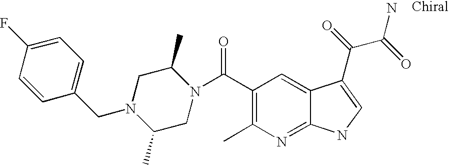 | 0.08 | B | 0.873 | 452 |
| 67 | 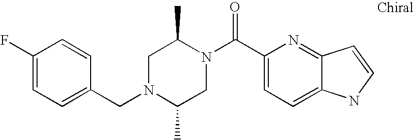 | 0.75 | B | 0.74 | 367 |
| 68 | 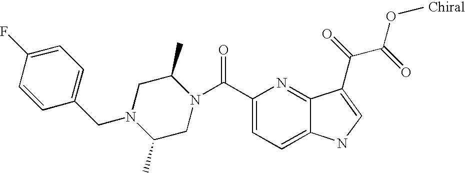 | 0.76 | B | 0.993 | 453 |
| 69 | 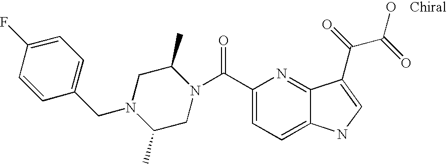 | 0.70 | B | 0.827 | 439 |
| 70 | 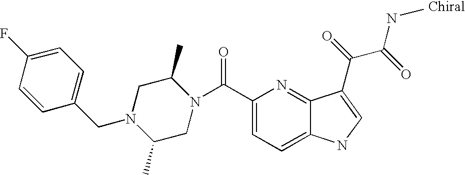 | 0.60 | B | 0.90 | 452 |
| 71 | 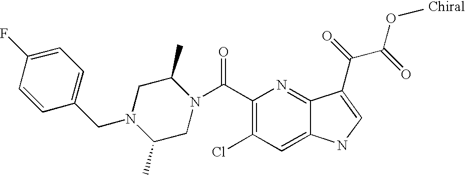 | 0.034 | B | 1.033 | 487 |
| 72 | 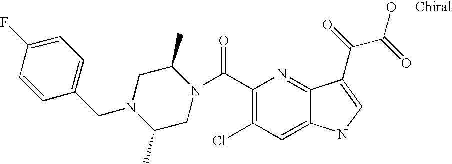 | 0.014 | B | 0.853 | 473 |
| 73 | 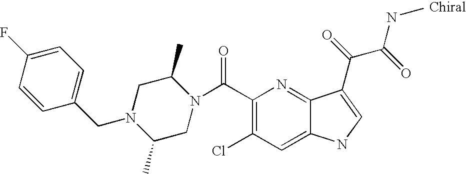 | 0.020 | B | 0.913 | 486 |
| 74 | 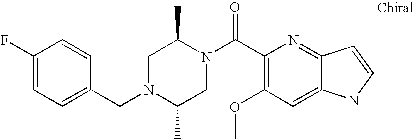 | 0.098 | B | 0.82 | 397 |
| 75 | 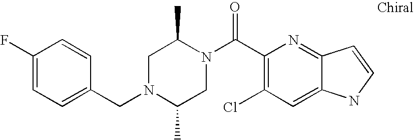 | 0.066 | B | 0.90 | 437 |
| 76 | 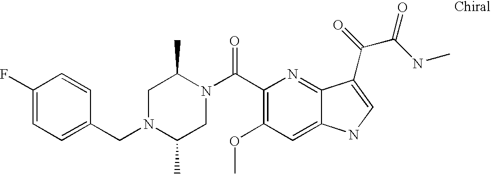 | 0.098 | B | 0.87 | 482 |
| 77 | 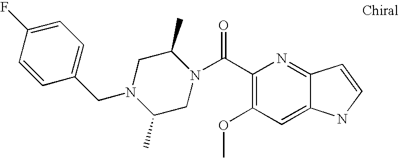 | 0.041 | B | 0.887 | 398 |

1. A compound of the formula: 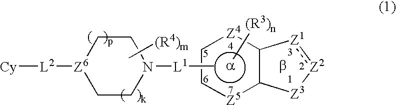
and the pharmaceutically acceptable salts thereof and prodrugs thereof, wherein: [symbol]
represents a single or double bond;
one of Z¹ and Z² is CQ or CR¹Q and the other of Z¹ and Z² is CR¹ or C(R¹)₂;
Q can be any of the groups that R¹ can represent, or Q can be WᵢC(═O)XⱼY, wherein
each of W and X is independently an alkylene, alkenylene, alkynylene, or heteroalkylene linker up to four atoms in length, each of which is optionally substituted with one or more C1-C4 alkyl, C1-C4 heteroalkyl, halo, CN, COOR, ═O, ═NR, ═NOR, ═N—CN, OR, and NR₂, wherein each R is independently H, C1-C4 alkyl, or C1-C4 heteroalkyl, and wherein two R can optionally cyclize to form a 3-7 membered ring containing 0-2 heteroatoms selected from N, O and S;
each of i and j is independently 0 or 1; and
Y is COR² or an isostere thereof;
Z³ is NR⁷ O, or S;
Z⁴ and Z⁵ are independently N, CH or CR³, or one of Z⁴ and Z⁵ can be a carbon to which L¹ is linked,
provided that at least one of Z⁴ and Z⁵ is N;
Z⁶ is N or CR⁵;
each of L¹ and L² is an alkylene, alkenylene, alkynylene, or heteroalkylene linker up to four atoms in length, which is optionally substituted with one or more C1-C4 alkyl, C1-C4 heteroalkyl, halo, CN, COOR, ═O, ═NR, ═NOR, ═N—CN, OR, or NR₂, wherein each R is independently H, C1-C4 alkyl, or C1-C4 heteroalkyl, and wherein two R can optionally cyclize to form a 3-7 membered ring containing 0-2 heteroatoms selected from N, O and S;
Cy is a cyclic group having 3-7 ring members that is substituted with 0-5 substituents R⁶, wherein two R⁶ substituents can form a ring that is fused to Cy, or
Cy can represent two cyclic groups having 3-7 ring members each, where both cyclic groups are bonded to a single atom of L² and where each of the two cyclic groups is optionally substituted with 0-5 substituents R⁶;
each R³ and R⁶ independently represents an optionally substituted C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or it can be halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-C12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
and wherein two R′ can be linked to form a 3-7 membered ring optionally containing up to three heteroatoms selected from N, O and S;
each R¹, R², R⁵, and R⁷ independently represents H or one of the groups set forth for R³;
each R⁴ represents one of the groups set forth for R³, or it can be ═CR′₂, ′O, ═N—CN, ═N—OR′, or ═NR′, and two R⁴ can be linked to form a fused ring, spiro-fused ring, or bridging ring having 3-7 members;
wherein each R′ is independently H, C1-C6 alkyl, C1-C6 heteroalkyl, C1-C6 acyl, C2-C6 heteroacyl, C6-C10 aryl or C5-C10 heteroaryl;
each of p and k is an integer from 0-2 wherein the sum of p and k is 0-3;
n is 0-2; and
m is 0-4.

2. The compound of claim 1 wherein [symbol] represents a double bond.

3. The compound of claim 2 wherein Q is —Wᵢ—COXⱼY.

4. The compound of claim 3 wherein Q is COXⱼCOR² and
wherein R² is OR, NR₂, SR, NRCONR₂, OCONR₂, or NRSO₂NR₂,
wherein each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O,
and wherein two R′ can be linked to form a 3-7 membered ring optionally containing up to three heteroatoms selected from N, O and S; and
X, if present, is alkylene.

5. The compound of claim 4 wherein j is 0.

6. The compound of claim 2, wherein Z¹ is CQ; and
Z² is CR¹,
wherein R¹ is H or is an optionally substituted C1-C8 alkyl, C1-C8 heteroalkyl, C1-C8 acyl, C2-C8 heteroacyl, C6-C10 aryl, C5-C12 heteroaryl, C7-C12 arylalkyl, or C6-C12 heteroarylalkyl group,
or R¹ can be halo, OR, NR₂, SR, SOR, SO₂R, SO₂NR₂, NRSO₂R, NRCONR₂, NRCOOR, NRCOR, CN, COOR, CONR₂, OOCR, COR, or NO₂, wherein each R is independently H, C1-C8 alkyl, or C1-C8 acyl, and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl,
and wherein two R′ can be linked to form a 3-7 membered ring optionally containing up to three heteroatoms selected from N, O and S.

7. The compound of claim 6, wherein Z² is CR′, wherein:
R¹ is H or C1-C4 alkyl optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl; and
Q is —COCOR², wherein
R² is OR, NR₂, NRCONR₂, or NRSO₂NR₂,
wherein each R is independently H, C1-C8 alkyl, C1-C8 alkenyl or C6-C10 aryl or the heteroatom-containing forms thereof,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl,
and wherein two R′ can be linked to form a 3-7 membered ring optionally containing up to three heteroatoms selected from N, O and S.

8. The compound of claim 2 wherein Z³ is NR⁷ and R⁷ is H or is optionally substituted C1-C8 alkyl, C2-C8 alkenyl, C2-C8 alkynyl, C6-C10 aryl, C7-C12 arylalkyl, C1-C8 acyl, C2-C8 heteroaryl, C1-C8 heteroalkyl, C2-C8 heteroalkenyl, C2-C8 heteroalkynyl, C6-C12 heteroalkylaryl, or is SOR, SO₂R, RCO, COOR, C1-C4-alkyl-COR, SO₃R, CONR₂, SO₂NR₂, CN, CF₃, NR₂, OR, C1-C4-alkyl-SR, C1-C4-alkyl-SOR, C1-C4-alkyl-SO₂R, C1-C4-alkyl-OCOR, C1-C4-alkyl-COOR, C1-C4-alkyl-CN, or C1-C4-alkyl-CONR₂, wherein
each R is independently H or C1-C8 alkyl, C1-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl.

9. The compound of claim 8 wherein R⁷ is H, or is optionally substituted alkyl, optionally substituted acyl, optionally substituted heteroacyl, OR, or NR₂, wherein each R is independently H, alkyl, alkenyl or aryl or heteroforms thereof.

10. The compound of claim 1 wherein both p and k are 1.

11. The compound of claim 1 wherein L¹ is CO, C═NOR or CH₂,
wherein R is H or C1-C8 alkyl or C1-C8 acyl or a heteroform of one of these.

12. The compound of claim 10 wherein L¹ is CO.

13. The compound of claim 1 wherein Z⁶ is N.

14. The compound of claim 1 wherein Z⁶ is CR⁵, wherein R⁵ is H, OR, NR₂, SR or halo, and wherein each R is independently H, C1-C8 alkyl, C1-C8 heteroalkyl, C1-C8 acyl, or C1-C8 heteroacyl, each of which is optionally substituted with one or more halo, CN, or C1-C4 alkoxy groups.

15. The compound of claim 1 wherein L² is an alkylene or heteroalkylene linker up to four atoms in length, which is optionally substituted with one or more C1-C4 alkyl, C1-C4 heteroalkyl, halo, CN, COOR, ═O, ═NR, ═NOR, ═N—CN, OR, or NR₂, wherein each R is independently H, C1-C4 alkyl, or C1-C4 heteroalkyl, and wherein two R can optionally cyclize to form a 3-7 membered ring containing 0-2 heteroatoms selected from N, O and S

16. The compound of claim 15 wherein L² is unsubstituted C1-C4 alkylene or is CHMe.

17. The compound of claim 12 wherein L² is CH₂.

18. The compound of claim 1, wherein Cy comprises an optionally substituted phenyl or optionally substituted 5-6 membered heteroaryl ring.

19. The compound of claim 18 wherein Cy is phenyl that is optionally substituted with 0-3 substituents selected from halo, C1-C4 alkyl, and C1-C4 heteroalkyl, and wherein two of these substituents can cyclize to form a fused 5-7 membered ring containing up to two heteroatoms selected from N, O and S.

20. The compound of claim 19 wherein Cy is phenyl which is unsubstituted or has a single substituent.

21. The compound of claim 17, wherein Cy is phenyl which is unsubstituted or has a single substituent.

22. The compound of claim 1 wherein R⁴ is an optionally substituted C1-C8 alkyl, C1-C8 heteroalkyl, C1-C8 acyl, C2-C8 heteroacyl, C6-C10 aryl, C5-C12 heteroaryl, C7-C12 arylalkyl, or C6-C12 heteroarylalkyl group, or it can be halo or ═CR′₂, ═O, ═N—CN, ═N—OR′, or ═NR′, and wherein two R⁴ can be linked to form a fused ring, spiro-fused ring, or bridging ring having 3-7 ring members,
which ring is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, and
wherein each R′ is independently H, C1-C6 alkyl, C1-C6 heteroalkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl.

23. The compound of claim 22 wherein each R⁴ is independently halo, C1-C4 alkyl, ═O, C1-C4 heteroalkyl, or C2-C8 heteroacyl.

24. The compound of claim 23 wherein m is 0, 1, or 2.

25. The compound of claim 21 wherein m is 0,
or wherein m is 2 and each R⁴ is C 1-C4 alkyl.

26. The compound of claim 1, wherein the azacyclic ring in formula (1) that includes Z⁶ is one of the following formulas: 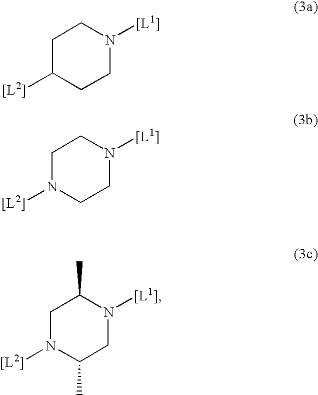
wherein [L¹] and [L²] indicate the point of attachment of the linkers L¹ and L² respectively in formula (1).

27. The compound of claim 23, wherein Z⁶ is N and the azacyclic ring which contains Z⁶ is 2R,5S-dimethylpiperazine when Z⁶ is defined as position 4,
or wherein the ring containing Z⁶ is 2,5-dimethylpiperidine having the same stereochemistry as the isomer of 2R,5S-dimethylpiperazine just described.

28. The compound of claim 2 wherein each R³ is independently halo, C1-C8 alkyl, C1-C8 heteroalkyl, C2-C8 heteroacyl, NRCOR, or NR₂, wherein each R is independently H, C1-C4 alkyl, C1-C8 acyl, or C1-C8 heteroacyl.

29. The compound of claim 28 wherein each R³ is independently C1-C4 alkyl, halo or C1-C4 alkoxy.

30. The compound of claim 29 wherein n is 0 or 2.

31. The compound of claim 29, wherein n is 1.

32. The compound of claim 1 having the formula (2a), (2b) or (2c): 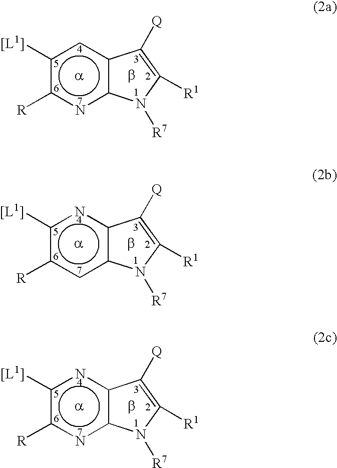
wherein [L¹] indicates the point of attachment of linker L¹ to the α ring;
R represents either H or R³;
and Q, R¹, R³, and R⁷ are as defined in claim 1.

33. The compound of claim 1 having the formula (4a): 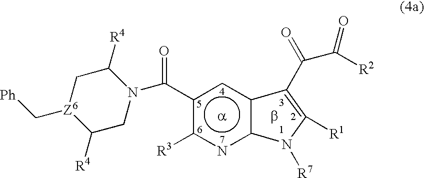
wherein Ph represents phenyl, optionally substituted with up to two groups selected from halo, C1-C4 alkyl, and C1-C4 alkoxy;
R¹ is H or C1-C4 alkyl;
R² is OR, NR₂, NRCONR₂, or NRSO₂NR₂,
wherein each R is independently H, C1-C8 alkyl, C1-C8 alkenyl or C1-C8 acyl or the heteroatom-containing forms thereof,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
R³ represents H, halo, C1-C4 alkyl, or C1-C4 alkoxy;
each R⁴ is independently H, C1-C4 alkyl, or ═O;
R⁷ is as defined in claim 1; and
Z⁶ is N or CR⁵, wherein R is H, OR, NR₂, SR or halo, and wherein each R is independently H, C1-C8 alkyl, C1-C8 heteroalkyl, C1-C8 acyl, or C1-C8 heteroacyl, each of which is optionally substituted with one or more halo, CN, or C1-C4 alkoxy groups.

34. The compound of claim 1 having the formula (4b): 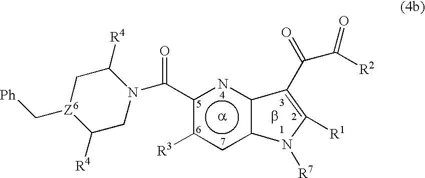
wherein Ph represents phenyl, optionally substituted with up to two groups selected from halo, C1-C4 alkyl, and C1-C4 alkoxy;
R¹ is H or C1-C4 alkyl;
R² is OR, NR₂, NRCONR₂, or NRSO₂NR₂,
wherein each R is independently H, C1-C8 alkyl, C1-C8 alkenyl or C1-C8 acyl or the heteroatom-containing forms thereof,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
R³ represents H, halo, C1-C4 alkyl, or C1-C4 alkoxy;
each R⁴ is independently H, C1-C4 alkyl, or ═O;
R⁷ is as defined in claim 1; and
Z⁶ is N or CR⁵, wherein R⁵ is H, OR, NR₂, SR or halo, and wherein each R is independently H, C1-C8 alkyl, C1-C8 heteroalkyl, C1-C8 acyl, or C1-C8 heteroacyl, each of which is optionally substituted with one or more halo, CN, or C1-C4 alkoxy groups.

35. The compound of claim 1 having the formula (4c): 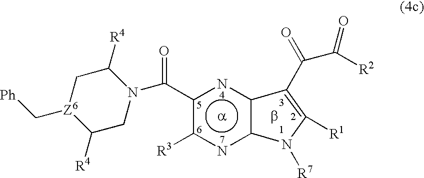
wherein Ph represents phenyl, optionally substituted with up to two groups selected from halo, C1-C4 alkyl, and C1-C4 alkoxy;
R¹ is H or C1-C4 alkyl;
R² is OR, NR₂, NRCONR₂, or NRSO₂NR₂,
wherein each R is independently H, C1-C8 alkyl, C1-C8 alkenyl or C1-C8 acyl or the heteroatom-containing forms thereof,
and wherein two R can be linked to form a 3-8 membered ring, optionally containing one or more N, O or S;
and wherein each R, and each ring formed by linking two R, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
wherein each R′ is independently H, C1-C6 alkyl, C1-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
R³ represents H, halo, C1-C4 alkyl, or C1-C4 alkoxy;
each R⁴ is independently H, C1-C4 alkyl, or ═O;
R⁷ is as defined in claim 1; and
Z⁶ is N or CR⁵, wherein R⁵ is H, OR, NR₂, SR or halo, and wherein each R is independently H, C1-C8 alkyl, C1-C8 heteroalkyl, C1-C8 acyl, or C1-C8 heteroacyl, each of which is optionally substituted with one or more halo, CN, or C1-C4 alkoxy groups.

36. The compound of claim 2, wherein L¹ is attached to the ring labeled α at position 6.

37. The compound of claim 2, wherein L¹ is attached to the ring labeled α at position 5.

38. The compound of claim 1, that is an acid addition salt formed by adding hydrochloric acid to a compound of formula (1).

39. The compound of claim 1 wherein the compound is selected from the group consisting of the compounds of formula (1) depicted in Examples 1-14.

40. The compound of claim 1, which is a compound in Table 1 or an acid addition salt thereof.

41. The compound of claim 1, which is a compound in FIG. 1a, 1b, 1c, 1d, or 1e, or an acid addition salt thereof.

42. A pharmaceutical composition for treating conditions characterized by excessive p38-α activity which composition comprises a therapeutically effective amount of at least one compound of claim 1 and at least one pharmaceutically acceptable excipient.

43. The composition of claim 42 which further contains an additional therapeutic agent.

44. The composition of claim 43 wherein said additional therapeutic agent is a corticosteroid, a monoclonal antibody, or an inhibitor of cell division.

45. A method to treat a condition characterized by excessive p38 kinase activity, comprising administering to a subject in need of such treatment a compound of claim 1, or a pharmaceutical composition thereof.

46. The method of claim 45 wherein said condition is a proinflammation response.

47. The method of claim 46 wherein said proinflammation response is multiple sclerosis, IBD, rheumatoid arthritis, rheumatoid spondylitis, osteoarthritis, gouty arthritis, other arthritic conditions, sepsis, septic shock, endotoxic shock, Gram-negative sepsis, toxic shock syndrome, asthma, adult respiratory distress syndrome, stroke, reperfusion injury, CNS injury, psoriasis, restenosis, cerebral malaria, chronic pulmonary inflammatory disease, chronic obstructive pulmonary disease, cystic fibrosis, silicosis, pulmonary sarcosis, bone fracture healing, a bone resorption disease, soft tissue damage, graft-versus-host reaction, Crohn's Disease, ulcerative colitis, Alzheimer's disease or pyresis.

48. The method of claim 47, wherein said proinflammation response is selected from rheumatoid arthritis, rheumatoid spondylitis, osteoarthritis, gouty arthritis, and other arthritic conditions.

49. The method of claim 45, wherein the condition is selected from rheumatoid arthritis, myelodysplasia, psoriasis, multiple myeloma, Hogkins and Non-Hodgkins lymphomas, renal carcinomas, and other cancers, metastatic bone disease, osteolytic lesions, osteoarthritis, osteoporosis and improper bone healing, and acute, chronic, or neuropathic pain.
